(19) United States
(12) Patent Application Publication
(10) Pub. No.: US 2023/0279433 A1
(43) Pub. Date: Sep. 7, 2023

(54) METHODS AND COMPOSITIONS FOR THE PRODUCTION OF ADENO-ASSOCIATED VIRUS

(71) Applicant: Oxford Biomedica Solutions LLC, Bedford, MA (US)

(72) Inventors: Marissa Stanvick, Bedford, MA (US); Richard Gilmore, Bedford, MA (US); Divya Tsiros, Bedford, MA (US)

(21) Appl. No.: 18/066,567

(22) Filed: Dec. 15, 2022

Related U.S. Application Data
(60) Provisional application No. 63/265,429, filed on Dec. 15, 2021.

(51) Int. Cl. C12N 15/86

(52) U.S. Cl. CPC C12N 15/86; C12N 2750/14152; C12N 2750/14143

(57) ABSTRACT

Provided herein are methods for the production of recombinant adeno-associated virus (rAAV) particles. These methods are particularly useful for the large-scale production of AAV particles.

### RELATED APPLICATIONS

This application claims priority to U.S. Provisional patent application Ser. No. 63/265,429, filed Dec. 15, 2021, the entire disclosure of which is hereby incorporated herein by reference.

### SEQUENCE LISTING

This application contains a sequence listing which has been submitted electronically in ST.26 format and is hereby incorporated by reference in its entirety (said ST.26 copy, created on Dec. 14, 2022, is named “HMW-152_SL_ST26.xml” and is 133,085 bytes in size).

### BACKGROUND

Gene therapy using adeno-associated virus (AAV) vectors has the potential to treat a wide variety of human disorders. AAV gene therapy requires dosages that can be as high as 1e15 vector genomes per kilogram bodyweight (vg/kg). Consequently, large-scale production processes are necessary in order to produce the large amounts required for commercial therapeutic use. In order for production processes to be commercially feasible, processes must be optimized to maximize AAV productivity.

Accordingly, there is a need in the art for novel AAV production methods that can provide for the consistent large-scale production of AAV.

### SUMMARY

The present disclosure provides methods for the production of rAAV particles. The methods generally comprise introducing into a mammalian cell a first polynucleotide comprising an rAAV genome to generate an AAV producer cell; and culturing the AAV producer cell in a culture medium containing dimethyl sulfoxide (DMSO) and/or culturing the AAV producer cell at an elevated temperature, to enhance the production of rAAV particles by the AAV producer cell. These methods are particularly useful for the large-scale production of rAAV particles.

Accordingly, in one aspect, the present disclosure provides a method for producing recombinant AAV (rAAV) particles, comprising: (a) introducing into a mammalian cell a first polynucleotide comprising an rAAV genome, to generate an AAV producer cell; (b) culturing the AAV producer cell in a first culture medium at a first temperature for a first period of time; and (c) culturing the AAV producer cell in a second culture medium at a second temperature for a second period of time, wherein the second temperature is about 38° C. to about 42° C., such that rAAV particles are produced by the AAV producer cell.

In certain embodiments, the second culture medium comprises an additive selected from the group consisting of dimethyl sulfoxide (DMSO), valproic acid or a salt thereof, propionic acid or a salt thereof, and butyric acid or a salt thereof. In certain embodiments, the additive is DMSO. In certain embodiments, the additive is valproic acid or a salt thereof. In certain embodiments, the additive is propionic acid or a salt thereof. In certain embodiments, the additive is butyric acid or a salt thereof.

In certain embodiments, the first temperature is about 30° C. to about 37° C., optionally about 37° C. In certain embodiments, the first temperature is about 38° C. to about 42° C., optionally about 39° C.

In certain embodiments, the first temperature is about 30° C. to about 37° C., optionally about 37° C.; and the second temperature is about 38° C. to about 42° C., optionally about 39° C. In certain embodiments, the first temperature is about 38° C. to about 42° C., optionally about 39° C.; and the second temperature is about 38° C. to about 42° C., optionally about 39° C.

In certain embodiments, the first and/or second culture medium has a pH of about 7.2. In certain embodiments, the first and/or second culture medium has a pH of about 6.8. In certain embodiments, the first and/or second culture medium has a pH of about 7.

In certain embodiments, the first culture medium has a pH of about 7.2; and the second culture medium has a pH of about 6.8. In certain embodiments, the first culture medium has a pH of about 7.2; and the second culture medium has a pH of about 7. In certain embodiments, the first culture medium has a pH of about 6.8; and the second culture medium has a pH of about 7.2. In certain embodiments, the first culture medium has a pH of about 6.8; and the second culture medium has a pH of about 7.

In certain embodiments, the second culture medium comprises about 0.1% (v/v) to about 5% (v/v) DMSO, optionally 0.5% (v/v) to about 3% (v/v) DMSO, optionally about 1.5% (v/v) DMSO. In certain embodiments, the first culture medium comprises DMSO, optionally about 0.1% (v/v) to about 5% (v/v) DMSO, optionally about 0.5% (v/v) to about 3% (v/v) DMSO, optionally about 1.5% (v/v) DMSO. In certain embodiments, the first and second culture medium comprise the same concentration of DMSO, optionally about 0.1% (v/v) to about 5% (v/v) DMSO, optionally about 0.5% (v/v) to about 3% (v/v) DMSO, optionally about 1.5% (v/v) DMSO.

In certain embodiments, the second culture medium comprises about 1 mM to about 10 mM valproic acid, optionally 2.5 mM to about 7.5 mM valproic acid. In certain embodiments, the first culture medium comprises about 1 mM to about 10 mM valproic acid, optionally 2.5 mM to about 7.5 mM valproic acid. In certain embodiments, the first and second culture medium comprise the same concentration of valproic acid, optionally about 1 mM to about 10 mM valproic acid, optionally 2.5 mM to about 7.5 mM valproic acid.

In certain embodiments, the second culture medium comprises about 1 mM to about 20 mM propionic acid, optionally 5 mM to about 15 mM propionic acid. In certain embodiments, the first culture medium comprises about 1 mM to about 20 mM propionic acid, optionally 5 mM to about 15 mM propionic acid. In certain embodiments, the first and second culture medium comprise the same concentration of propionic acid, optionally about 1 mM to about 20 mM propionic acid, optionally 5 mM to about 15 mM propionic acid.

In certain embodiments, the second culture medium comprises about 1 mM to about 10 mM butyric acid, optionally 2.5 mM to about 7.5 mM butyric acid. In certain embodiments, the first culture medium comprises about 1 mM to about 10 mM butyric acid, optionally 2.5 mM to about 7.5 mM butyric acid. In certain embodiments, the first and second culture medium comprise the same concentration of butyric acid, optionally about 1 mM to about 10 mM butyric acid, optionally 2.5 mM to about 7.5 mM butyric acid.

In certain embodiments, prior to introduction of the first polynucleotide into the mammalian cell, the mammalian cell is cultured in a third culture medium at a third temperature for a third period of time.

In certain embodiments, the third culture medium comprises an additive selected from the group consisting of dimethyl sulfoxide (DMSO), valproic acid or a salt thereof, propionic acid or a salt thereof, and butyric acid or a salt thereof. In certain embodiments, the third culture medium comprises about 0.1% (v/v) to about 5% (v/v) DMSO, optionally about 0.5% (v/v) to about 3% (v/v) DMSO, optionally about 1.5% (v/v) DMSO.

In certain embodiments, the first, second, and third culture medium comprise the same concentration of DMSO, optionally about 0.1% (v/v) to about 5% (v/v) DMSO, optionally about 0.5% (v/v) to about 3% (v/v) DMSO, optionally about 1.5% (v/v) DMSO.

In certain embodiments, the third temperature is about 30° C. to about 37° C., optionally about 37° C.

In certain embodiments, third period of time is about 0.5 to about 3 hours. In certain embodiments, third period of time is about 0.5 hours, about 1 hour, about 1.5 hours, or about 2 hours.

In certain embodiments, the first period of time is about 0 to about 5 hours. In certain embodiments, the first period of time is about 0.5 hours, about 1 hour, about 1.5 hours, or about 2 hours. In certain embodiments, first period of time is about 2 hours. In certain embodiments, first period of time is about 0.5 hours.

In certain embodiments, the second period of time is about 1 to about 100 hours. In certain embodiments, the second period of time is about 48 to about 75 hours, optionally about 65 to about 75 hours.

In another aspect, the present disclosure provides a method for producing recombinant AAV (rAAV) particles, comprising: (a) culturing a mammalian cell in a culture medium comprising about 0.1% (v/v) to about 5% (v/v) DMSO, optionally 1.5% (v/v) DMSO, at a temperature of about 30° C. to about 37° C., optionally 37° C., for about 0.5 to about 3 hours, optionally 2 hours; (b) introducing into the mammalian cell a first polynucleotide comprising an rAAV genome, to generate an AAV producer cell; (c) culturing the AAV producer cell in culture medium comprising about 0.1% (v/v) to about 5% (v/v) DMSO, optionally 1.5% (v/v) DMSO, at a temperature of about 30° C. to about 37° C., optionally 37° C., for about 0.5 to about 2 hours; and (d) culturing the AAV producer cell in a culture medium comprising about 0.1% (v/v) to about 5% (v/v) DMSO, optionally 1.5% (v/v) DMSO, at a temperature of about 38° C. to about 42° C., optionally 39° C., for about 48 to about 75 hours, optionally 70 hours, such that rAAV particles are produced by the AAV producer cell.

In certain embodiments, step (c) is cultured for about 0.5 hours. In certain embodiments, step (c) is cultured for about 1 hour. In certain embodiments, step (c) is cultured for about 1.5 hours. In certain embodiments, step (c) is cultured for about 2 hours.

In certain embodiments, the mammalian cell is a mammalian cell selected from the group consisting of a COS cell, a CHO cell, a BHK cell, an MDCK cell, an HEK293 cell, an HEK293T cell, a HeLa cells, an NS0 cell, a PER.C6 cell, a VERO cell, a CRL7030 cell, an HsS78Bst cell, a HeLa cell, an NIH 3T3 cell, a HepG2 cell, an SP210 cell, an R1.1 cell, a B-W cell, an L-M cell, a BSC1 cell, a BSC40 cell, a YB/20 cell, and a BMT10 cell, optionally a cell that can be grown in suspension culture, optionally an HEK293 cell or an HEK293T cell that can be grown in suspension culture, optionally HEK293F.

In certain embodiments, a second polynucleotide encoding an AAV capsid protein, a third polynucleotide encoding an AAV Rep protein, and/or a fourth polynucleotide encoding one or more helper virus genes is introduced into the mammalian cell together with the first polynucleotide.

In certain embodiments, a second polynucleotide encoding an AAV capsid protein, a third polynucleotide encoding an AAV Rep protein, and a fourth polynucleotide encoding one or more helper genes are introduced into the mammalian cell together with the first polynucleotide.

In certain embodiments, the first, second, and/or third polynucleotide is comprised within a nucleic acid vector. In certain embodiments, the first and second polynucleotide are comprised within the same nucleic acid vector. In certain embodiments, the first, second, and third polynucleotide are comprised within the same nucleic acid vector. In certain embodiments, the first, second, third, and fourth polynucleotide are comprised within the same nucleic acid vector. In certain embodiments, the second, third, and fourth polynucleotide are comprised within the same nucleic acid vector.

In certain embodiments, the nucleic acid vector is a plasmid or a minimal DNA vector.

In certain embodiments, the polynucleotide(s) or nucleic acid vector(s) are introduced into the mammalian cell in step (a) by transfection; optionally wherein the transfection is mediated by a cationic polymer, optionally polyethylenimine.

In certain embodiments, the method further comprises purifying and formulating the AAV particles for administration to a human subject.

In certain embodiments, the AAV is a recombinant AAV (rAAV) comprising an rAAV genome.

In certain embodiments, the rAAV genome comprises a transgene encoding a polypeptide, miRNA, shRNA, siRNA, antisense RNA, gRNA, antagomir, miRNA sponge, RNA aptazyme, RNA aptamer, lncRNA, ribozyme, or mRNA.

In certain embodiments, the rAAV genome comprises a transgene encoding a protein selected from the group consisting of phenylalanine hydroxylase (PAH), glucose-6-phosphatase (G6Pase), iduronate-2-sulfatase (I2S), arylsulfatase A (ARSA), frataxin (FXN), and an antibody having specificity for complement component 5 (C5). In certain embodiments, the transgene encodes a protein which is not selected from the group consisting of phenylalanine hydroxylase (PAH), arylsulfatase A (ARSA), iduronate 2-sulfatase (I2S), and an anti-complement component 5 (C5) antibody.

In certain embodiments, the rAAV genome comprises a nucleotide sequence that is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 50, 51, 52, 53, or 54.

In certain embodiments, the rAAV genome further comprises a 5′ inverted terminal repeat (5′ ITR) nucleotide sequence 5′ of the transgene, and a 3′ inverted terminal repeat (3′ ITR) nucleotide sequence 3′ of the transgene. In certain embodiments, the 5′ ITR nucleotide sequence is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 39, 41, or 42, and/or the 3′ ITR nucleotide sequence is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 40, 43, or 44.

In certain embodiments, the rAAV genome comprises a nucleotide sequence that is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 55, 56, 57, 58, or 59.

In certain embodiments, the rAAV comprises an AAV capsid comprising an AAV capsid protein.

In certain embodiments, the AAV capsid protein is selected from the group consisting of AAV1, AAV2, AAV3, AAV4, AAV5, AAV6, AAV7, AAV8, AAV9, AAV-DJ, AAV-LK03, NP59, VOY101, VOY201, VOY701, VOY801, VOY1101, AAVPHP.N, AAVPHP.A, AAVPHP.B, PHP.B2, PHP.B3, G2A3, G2B4, G2B5, and PUP.S.

In certain embodiments, the AAV capsid protein comprises an amino acid sequence that is at least 85% identical to the amino acid sequence of amino acids 203-736 of SEQ ID NO: 1, 2, 3, 4, 5, 6, 7, 8, 9, 10, 11, 12, 13, 15, 16, or 17. In certain embodiments, the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, (a) the amino acid in the capsid protein corresponding to amino acid 626 of SEQ ID NO: 16 is G, and the amino acid in the capsid protein corresponding to amino acid 718 of SEQ ID NO: 16 is G; (b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (c) the amino acid in the capsid protein corresponding to amino acid 505 of SEQ ID NO: 16 is R, and the amino acid in the capsid protein corresponding to amino acid 687 of SEQ ID NO: 16 is R; (d) the amino acid in the capsid protein corresponding to amino acid 346 of SEQ ID NO: 16 is A, and the amino acid in the capsid protein corresponding to amino acid 505 of SEQ ID NO: 16 is R; or (e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the AAV capsid protein comprises the amino acid sequence of amino acids 203-736 of SEQ ID NO: 1, 2, 3, 4, 5, 6, 7, 8, 9, 10, 11, 12, 13, 15, 16, or 17.

In certain embodiments, the AAV capsid protein does not comprise an amino acid sequence that is at least 85% identical to the amino acid sequence of amino acids 203-736 of SEQ ID NO: 1, 2, 3, 4, 5, 6, 7, 8, 9, 10, 11, 12, 13, 15, 16, or 17. In certain embodiments, the AAV capsid protein does not comprise an amino acid sequence that is at least 85% identical to the amino acid sequence of amino acids 203-736 of SEQ ID NO: 1, 2, 3, 4, 5, 6, 7, 8, 9, 10, 11, 12, 13, 15, 16, or 17, wherein the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the AAV capsid protein does not comprise the amino acid sequence of amino acids 203-736 of SEQ ID NO: 1, 2, 3, 4, 5, 6, 7, 8, 9, 10, 11, 12, 13, 15, 16, or 17.

In certain embodiments, the AAV capsid protein comprises an amino acid sequence that is at least 85% identical to the amino acid sequence of amino acids 138-736 of SEQ ID NO: 1, 2, 3, 4, 5, 6, 7, 8, 9, 10, 11, 12, 13, 15, 16, or 17. In certain embodiments, the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, (a) the amino acid in the capsid protein corresponding to amino acid 626 of SEQ ID NO: 16 is G, and the amino acid in the capsid protein corresponding to amino acid 718 of SEQ ID NO: 16 is G; (b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (c) the amino acid in the capsid protein corresponding to amino acid 505 of SEQ ID NO: 16 is R, and the amino acid in the capsid protein corresponding to amino acid 687 of SEQ ID NO: 16 is R; (d) the amino acid in the capsid protein corresponding to amino acid 346 of SEQ ID NO: 16 is A, and the amino acid in the capsid protein corresponding to amino acid 505 of SEQ ID NO: 16 is R; or (e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the AAV capsid protein comprises the amino acid sequence of amino acids 138-736 of SEQ ID NO: 1, 2, 3, 4, 5, 6, 7, 9, 10, 11, 12, 13, 15, 16, or 17.

In certain embodiments, the AAV capsid protein does not comprise an amino acid sequence that is at least 85% identical to the amino acid sequence of amino acids 138-736 of SEQ ID NO: 1, 2, 3, 4, 5, 6, 7, 8, 9, 10, 11, 12, 13, 15, 16, or 17. In certain embodiments, the AAV capsid protein does not comprise an amino acid sequence that is at least 85% identical to the amino acid sequence of amino acids 138-736 of SEQ ID NO: 1, 2, 3, 4, 5, 6, 7, 8, 9, 10, 11, 12, 13, 15, 16, or 17, wherein the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the AAV capsid protein does not comprise the amino acid sequence of amino acids 138-736 of SEQ ID NO: 1, 2, 3, 4, 5, 6, 7, 9, 10, 11, 12, 13, 15, 16, or 17.

In certain embodiments, the AAV capsid protein comprises an amino acid sequence that is at least 85% identical to the amino acid sequence of amino acids 1-736 of SEQ ID NO: 1, 2, 3, 4, 5, 6, 7, 8, 9, 10, 11, 12, 13, 15, 16, or 17. In certain embodiments, the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, (a) the amino acid in the capsid protein corresponding to amino acid 2 of SEQ ID NO: 16 is T, and the amino acid in the capsid protein corresponding to amino acid 312 of SEQ ID NO: 16 is Q; (b) the amino acid in the capsid protein corresponding to amino acid 65 of SEQ ID NO: 16 is I, and the amino acid in the capsid protein corresponding to amino acid 626 of SEQ ID NO: 16 is Y; (c) the amino acid in the capsid protein corresponding to amino acid 77 of SEQ ID NO: 16 is R, and the amino acid in the capsid protein corresponding to amino acid 690 of SEQ ID NO: 16 is K; (d) the amino acid in the capsid protein corresponding to amino acid 119 of SEQ ID NO: 16 is L, and the amino acid in the capsid protein corresponding to amino acid 468 of SEQ ID NO: 16 is S; (e) the amino acid in the capsid protein corresponding to amino acid 626 of SEQ ID NO: 16 is G, and the amino acid in the capsid protein corresponding to amino acid 718 of SEQ ID NO: 16 is G; (f) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (g) the amino acid in the capsid protein corresponding to amino acid 505 of SEQ ID NO: 16 is R, and the amino acid in the capsid protein corresponding to amino acid 687 of SEQ ID NO: 16 is R; (h) the amino acid in the capsid protein corresponding to amino acid 346 of SEQ ID NO: 16 is A, and the amino acid in the capsid protein corresponding to amino acid 505 of SEQ ID NO: 16 is R; or (i) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the AAV capsid protein comprises the amino acid sequence of amino acids 1-736 of SEQ ID NO: 1, 2, 3, 4, 5, 6, 7, 8, 9, 10, 11, 12, 13, 15, 16, or 17.

In certain embodiments, the AAV capsid protein does not comprise an amino acid sequence that is at least 85% identical to the amino acid sequence of amino acids 1-736 of SEQ ID NO: 1, 2, 3, 4, 5, 6, 7, 8, 9, 10, 11, 12, 13, 15, 16, or 17. In certain embodiments, the AAV capsid protein does not comprise an amino acid sequence that is at least 85% identical to the amino acid sequence of amino acids 1-736 of SEQ ID NO: 1, 2, 3, 4, 5, 6, 7, 8, 9, 10, 11, 12, 13, 15, 16, or 17, wherein the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the AAV capsid protein does not comprise the amino acid sequence of amino acids 1-736 of SEQ ID NO: 1, 2, 3, 4, 5, 6, 7, 8, 9, 10, 11, 12, 13, 15, 16, or 17.

### BRIEF DESCRIPTION OF DRAWINGS

FIG. 1 depicts plots showing the predicted relationship between tested parameters (on x-axis) and VG titer (in vg/L) and capsid yield (in capsid/L) (on y-axis). Tested parameters include temperature (“Temp”), pH, percent level of dissolved oxygen (DO), and power per unit volume (P/V).

FIG. 2 depicts plots showing the predicted relationship between tested parameters (on x-axis) and VG titer (in vg/L) and capsid yield (in capsid/L) (on y-axis). Tested parameters include pH and temperature (“Temp”).

FIGS. 3A-3C are histograms of vector genome (VG) titers (FIG. 3A), capsid yield (FIG. 3B) and calculated percent full capsids (“Calculated % Full”; FIG. 3C) that were measured from crude lysates of AAV producer cells cultured in the indicated pH and temperature conditions that were set at two hours post-transfection. In FIG. 3A, VG titer is presented in vector genomes per liter (vg/L); in FIG. 3B, capsid yield is presented in capsids per liter (capsids/L); and in FIG. 3C calculated percent of full capsids is presented in percentage on the y-axis.

FIGS. 4A-4B are histograms of vector genome (VG) titers (FIG. 4A) and calculated percent full capsids (“% Full”; FIG. 4B) that were measured from crude lysates of AAV producer cells cultured in media supplemented with the indicated additives at the indicated concentrations, wherein the additives were supplemented two hours post-transfection. In FIG. 4A, VG titer is presented in vector genomes per liter (vg/L), and in FIG. 4B calculated percent of full capsids is presented in percentage on the y-axis. The control condition is AAV production without the supplementation of any additive two hours post-transfection. The concentrations of valproic acid, propionic acid, butyric acid, and DMSO that were supplemented are described in Table 5. Each condition was performed in duplicate.

FIGS. 5A-5C are histograms of vector genome (VG) titers (FIG. 5A), capsid yield (FIG. 5B) and calculated percent full capsids (“Calculated % Full”; FIG. 5C) that were measured from crude lysates of AAV producer cells cultured in media supplemented with 1.5% (v/v) DMSO, wherein the DMSO was supplemented at the indicated times (e.g., 2 hours pre-transfection (“Pre TFX”); at transfection (“At TFX”); and various times post-transfection (“Post TFX”). In FIG. 5A, VG titer is presented in vector genomes per liter (vg/L); in FIG. 5B, capsid yield is presented in capsids per liter (capsids/L); and in FIG. 5C calculated percent of full capsids is presented in percentage on the y-axis. Each condition was performed in duplicate.

FIGS. 6A-6C are histograms of vector genome (VG) titers (FIG. 6A), capsid yield (FIG. 6B) and calculated percent full capsids (FIG. 6C) that were measured from crude lysates of AAV producer cells cultured in the indicated temperature conditions that were set at two hours post-transfection. In FIG. 6A, VG titer is presented in vector genomes per liter (vg/L); in FIG. 6B, capsid yield is presented in capsids per liter (capsids/L); and in FIG. 6C calculated percent of full capsids is presented in percentage on the y-axis. The results for two different representative AAV constructs are shown.

FIGS. 7A-7C are histograms of vector genome (VG) titers (FIG. 7A), capsid yield (FIG. 7B) and calculated percent full capsids (FIG. 7C) that were measured from crude lysates of AAV producer cells cultured in the indicated pH and temperature conditions that were set at two hours post-transfection. In FIG. 7A, VG titer is presented in vector genomes per liter (vg/L); in FIG. 7B, capsid yield is presented in capsids per liter (capsids/L); and in FIG. 7C calculated percent of full capsids is presented in percentage on the y-axis. The results for two different representative AAV constructs are shown.

FIGS. 8A-8C are histograms of vector genome (VG) titers (FIG. 8A), capsid yield (FIG. 8B) and calculated percent full capsids (FIG. 8C) that were measured from crude lysates of AAV producer cells cultured in the indicated pH and temperature conditions that were set at 2 hours post-transfection, and with DMSO supplementation 120 minutes post-transfection or 30 minutes post-transfection (“30 m DMSO”). In FIG. 8A, VG titer is presented in vector genomes per liter (vg/L); in FIG. 8B, capsid yield is presented in capsids per liter (capsids/L); and in FIG. 8C calculated percent of full capsids is presented in percentage on the y-axis.

FIGS. 9A-9C are histograms of vector genome (VG) titers (FIG. 9A), capsid yield (FIG. 9B) and calculated percent full capsids (FIG. 9C) that were measured from crude lysates of AAV producer cells cultured in the indicated DMSO conditions that were set at two hours post-transfection. In FIG. 9A, VG titer is presented in vector genomes per liter (vg/L); in FIG. 9B, capsid yield is presented in capsids per liter (capsids/L); and in FIG. 9C calculated percent of full capsids is presented in percentage on the y-axis.

FIGS. 10A-10C are histograms of vector genome (VG) titers (FIG. 10A), capsid yield (FIG. 10B) and calculated percent full capsids (FIG. 10C) that were measured from crude lysates of AAV producer cells cultured in the indicated DMSO conditions that were set at two hours post-transfection. In FIG. 10A, VG titer is presented in vector genomes per liter (vg/L); in FIG. 10B, capsid yield is presented in capsids per liter (capsids/L); and in FIG. 10C calculated percent of full capsids is presented in percentage on the y-axis.

### DETAILED DESCRIPTION

The present disclosure provides methods for the improved production of rAAV particles. The methods generally comprise introducing into a mammalian cell a first polynucleotide comprising an rAAV genome to generate an AAV producer cell; and culturing the AAV producer cell in a culture medium containing dimethyl sulfoxide (DMSO) and/or culturing the AAV producer cell at an elevated temperature, to enhance the production of rAAV particles by the AAV producer cell. These methods are particularly useful for the large-scale production of rAAV particles.

### I. Definitions

As used herein, the term “recombinant adeno-associated virus” or “rAAV” refers to an adeno-associated virus (AAV) comprising a genome lacking functional rep and cap genes.

As used herein, the term “cap gene” refers to a nucleic acid sequence that encodes an AAV capsid protein.

As used herein, the term “rAAV genome” refers to a nucleic acid molecule comprising the genome sequence of an rAAV. The skilled artisan will appreciate that where an rAAV genome comprises a transgene, the rAAV genome can be in the sense or antisense orientation relative to the direction of transcription of the transgene.

As used herein, the term “editing genome” refers to a recombinant AAV genome that is capable of integrating an editing element (e.g., one or more nucleotides or an internucleotide bond) via homologous recombination into a target locus to correct a genetic defect in a target gene. The skilled artisan will appreciate that the portion of an editing genome comprising the 5′ homology arm, editing element, and 3′ homology arm can be in the sense or antisense orientation relative to the target locus.

As used herein, the term “editing element” refers to the portion of an editing genome that when integrated at a target locus modifies the target locus. An editing element can mediate insertion, deletion, or substitution of one or more nucleotides at the target locus. As used herein, the term “target locus” refers to a region of a chromosome or an internucleotide bond (e.g., a region or an internucleotide bond of a target gene) that is modified by an editing element.

As used herein, the term “homology arm” refers to a portion of an editing genome positioned 5′ or 3′ of an editing element that is substantially identical to the genome flanking a target locus.

As used herein, the “percentage identity” between two nucleotide sequences or between two amino acid sequences is calculated by multiplying the number of matches between the pair of aligned sequences by 100, and dividing by the length of the aligned region, including internal gaps. Identity scoring only counts perfect matches and does not consider the degree of similarity of amino acids to one another. Note that only internal gaps are included in the length, not gaps at the sequence ends.

As used herein, the term “coding sequence” refers to the portion of a complementary DNA (cDNA) that encodes a polypeptide, starting at the start codon and ending at the stop codon. A gene may have one or more coding sequences due to alternative splicing, alternative translation initiation, and variation within the population. A coding sequence may be wild-type or a non-naturally occurring variant (e.g., a codon optimized variant).

As used herein, the term “transcriptional regulatory element” or “TRE” refers to a cis-acting nucleotide sequence, for example, a DNA sequence, that regulates (e.g., controls, increases, or reduces) transcription of an operably linked nucleotide sequence by an RNA polymerase to form an RNA molecule. A TRE relies on one or more trans-acting molecules, such as transcription factors, to regulate transcription. Thus, one TRE may regulate transcription in different ways when it is in contact with different trans-acting molecules, for example, when it is in different types of cells. A TRE may comprise one or more promoter elements and/or enhancer elements. A skilled artisan would appreciate that the promoter and enhancer elements in a gene may be close in location, and the term “promoter” may refer to a sequence comprising a promoter element and an enhancer element. Thus, the term “promoter” does not exclude an enhancer element in the sequence. The promoter and enhancer elements do not need to be derived from the same gene or species, and the sequence of each promoter or enhancer element may be either identical or substantially identical to the corresponding endogenous sequence in the genome.

As used herein, the term “operably linked” is used to describe the connection between a TRE and a coding sequence to be transcribed. Typically, gene expression is placed under the control of a TRE comprising one or more promoter and/or enhancer elements. The coding sequence is “operably linked” to the TRE if the transcription of the coding sequence is controlled or influenced by the TRE. The promoter and enhancer elements of the TRE may be in any orientation and/or distance from the coding sequence, as long as the desired transcriptional activity is obtained. In certain embodiments, the TRE is upstream from the coding sequence.

As used herein, the term “polyadenylation sequence” refers to a DNA sequence that when transcribed into RNA constitutes a polyadenylation signal sequence. The polyadenylation sequence can be native or exogenous. The exogenous polyadenylation sequence can be a mammalian or a viral polyadenylation sequence (e.g., an SV40 polyadenylation sequence).

As used herein, “exogenous polyadenylation sequence” refers to a polyadenylation sequence not identical or substantially identical to the endogenous polyadenylation sequence of a transgene. In certain embodiments, an exogenous polyadenylation sequence is a polyadenylation sequence of a gene different from the transgene, but within the same species (e.g., human). In certain embodiments, an exogenous polyadenylation sequence is a polyadenylation sequence of a different organism (e.g., a virus).

As used herein, the term “about,” when in reference to a value or parameter herein, includes a variability of ±1% of the value or parameter. For example, when referring to a pH value, “about” refers to a range that includes the value 1% below the referenced value, and the value 1% above the referenced value. Thus, a pH of about 10 refers to a pH that encompasses a pH of 9.9 to a pH of 10.1, inclusive.

As used herein, the term “AAV producer cell” refers to a cell which is generated by the introduction of a polynucleotide comprising an rAAV genome into a mammalian cell, and is thereby capable of producing an AAV. In certain embodiments, the polynucleotide is comprised within a nucleic acid vector.

### II. Methods for Producing Recombinant Adeno-Associated Virus Particles

The present disclosure is directed to methods for the improved production of recombinant adeno-associated virus (rAAV) particles. AAV manufacturing comprises an upstream production process that generally comprises expanding mammalian cells to an appropriate cell density, introducing one or more polynucleotides into the expanded cells to generate AAV producer cells, culturing the AAV producer cells under conditions to produce rAAV particles, and harvesting and lysing the AAV producer cells for subsequent recovery of the rAAV particles. After recovery of the rAAV particles, downstream production processes ensure the sufficient purification of the rAAV particles from contaminants.

After generating the AAV producer cells, the AAV producer cells are generally cultured for a certain period of time to maximize the amount of rAAV particles that are produced. Provided herein are methods that comprise changing one or more culture parameters and/or components of the culture medium in which the AAV producer cell is cultured. It has been found that changing one or more culture parameters and/or components of the culture medium after the AAV producer cell has been cultured for a first period of time (e.g., post-transfection changing of the one or more culture parameters and/or components) results in improved productivity, e.g., improved titers of rAAV particles obtained. As such, methods of the present disclosure comprise culturing the AAV producer cell for a first period of time under a first set of conditions, and then culturing the AAV producer cell for a second period of time under a second set of conditions.

### Culture Media and Methods

In certain embodiments, the methods described herein comprise: (a) introducing into a mammalian cell a first polynucleotide comprising an rAAV genome, to generate an AAV producer cell; (b) culturing the AAV producer cell in a first culture medium at a first temperature for a first period of time; and (c) culturing the AAV producer cell in a second culture medium at a second period of time, such that rAAV particles are produced by the AAV producer cell.

The first culture medium and the second culture medium can comprise any medium known to those of skill in the art that is suitable for the introduction of polynucleotides into mammalian cells. Examples of a suitable first culture medium and/or second culture medium include, without limitation, HuMEC Basal Serum free Medium, KNOCKOUT™ CTS™ XenoFREE ESC/iPSC Medium, STEMPRO™-34 SFM Medium, STEMPRO™ NSC Medium, ESSENTIAL™-8 Medium, Medium 254, Medium 106, Medium 131, Medium 154, Medium 171, Medium 200, Medium 231, HepatoZYME-SFM (Thermo Fisher), Human Endothelial-SFM, GIBCO® FREESTYLE™ 293 Expression Medium, Medium 154CF/PRF, Medium 154C, Medium 154CF, Medium 200PRF, ESSENTIAL™-6 Medium, STEMPRO™-34 Medium, GIBCO® Astrocyte Medium, AIM V® Medium CTS™, AMINOMAX™ C-100 Basal Medium, AMINOM AX™-II Complete Medium, CD FORTICHO™ Medium, CD CHO ACT Medium, CHO—S-SFM Medium, GIBCO® FREESTYLE™ CHO Expression Medium, CD OPTICHO™ Medium, CD CHO Medium, CD DG44 Medium, SF-900™ Medium, EXPI293™ Expression Medium, LHC Basal Medium, LHC-8 Medium, 293 SFM Medium, CD 293 Medium, AEM Growth Medium, PER. C6® Cell Medium, AIM V® Medium, EXPILIFE® Medium, Keratinocyte-SFM Medium, LHC Medium, LHC-8 Medium, LHC-9 Medium, and modifications thereof.

One or more additives may be used to supplement the first culture medium and/or the second culture medium. In certain embodiments, the additive is supplemented to optimize and/or improve the production efficiency (e.g., production of an rAAV particle) of the AAV producer cell. Additives that may be used to supplement the first culture medium and/or the second culture medium include, without limitation, valproic acid or a salt thereof (e.g., sodium valproate), propionic acid or a salt thereof (e.g., sodium propionate), butyric acid or a salt thereof (e.g., sodium butyrate), dimethyl sulfoxide (DMSO), trichostatin A, lithium acetate, caffeine, hydroxyurea, and nocodazole.

In certain embodiments, the first culture medium and the second culture medium are substantially identical, i.e., they contain the same components. In certain embodiments, the first culture medium and the second culture medium are substantially different. In certain embodiments, the first culture medium does not comprise an additive and the second culture medium comprises an additive. In certain embodiments, the first culture medium comprises an additive and the second culture medium does not comprise an additive. In certain embodiments, the first culture medium comprises a first additive, and the second culture medium comprises a second additive, wherein the first and second additive are different.

In certain embodiments, the first culture medium comprises DMSO and the second culture medium does not comprise DMSO. In certain embodiments, the first culture medium does not comprise DMSO and the second culture medium comprises DMSO. In certain embodiments, the first culture medium and second culture medium each comprise up to about 5% (v/v) DMSO, e.g., no DMSO, about 0.1% (v/v), about 0.2% (v/v), about 0.3% (v/v), about 0.4% (v/v), about 0.5% (v/v), about 0.6% (v/v), about 0.7% (v/v), about 0.8% (v/v), about 0.9% (v/v), about 1% (v/v), about 1.1% (v/v), about 1.2% (v/v), about 1.3% (v/v), about 1.4% (v/v), about 1.5% (v/v), about 1.6% (v/v), about 1.7% (v/v), about 1.8% (v/v), about 1.9% (v/v), about 2% (v/v), about 2.1% (v/v), about 2.2% (v/v), about 2.3% (v/v), about 2.4% (v/v), about 2.5% (v/v), about 2.6% (v/v), about 2.7% (v/v), about 2.8% (v/v), about 2.9% (v/v), about 3% (v/v), about 3.1% (v/v), about 3.2% (v/v), about 3.3% (v/v), about 3.4% (v/v), about 3.5% (v/v), about 3.6% (v/v), about 3.7% (v/v), about 3.8% (v/v), about 3.9% (v/v), about 4% (v/v), about 4.1% (v/v), about 4.2% (v/v), about 4.3% (v/v), about 4.4% (v/v), about 4.5% (v/v), about 4.6% (v/v), about 4.7% (v/v), about 4.8% (v/v), about 4.9% (v/v), or about 5% (v/v) DMSO. In certain embodiments, the first culture medium comprises about 0.1% (v/v) to about 5% (v/v) DMSO. In certain embodiments, the first culture medium comprises about 0.5% (v/v) to about 5% (v/v) DMSO. In certain embodiments, the first culture medium comprises about 1.5% (v/v) DMSO. In certain embodiments, the second culture medium comprises about 0.1% (v/v) to about 5% (v/v) DMSO. In certain embodiments, the second culture medium comprises about 0.5% (v/v) to about 5% (v/v) DMSO. In certain embodiments, the second culture medium comprises about 1.5% (v/v) DMSO.

In certain embodiments, the first culture medium comprises propionic acid or a salt thereof (e.g., sodium propionate) and the second culture medium does not comprise propionic acid or a salt thereof (e.g., sodium propionate). In certain embodiments, the first culture medium does not comprise propionic acid or a salt thereof (e.g., sodium propionate) and the second culture medium comprises propionic acid or a salt thereof (e.g., sodium propionate). In certain embodiments, the first culture medium and second culture medium each comprise up to about 20 mM propionic acid or a salt thereof (e.g., sodium propionate), e.g., no propionic acid or a salt thereof (e.g., sodium propionate), about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 7.5 mM, about 8 mM, about 8.5 mM, about 9 mM, about 9.5 mM, about 10 mM, about 10.5 mM, about 11 mM, about 11.5 mM, about 12 mM, about 12.5 mM, about 13 mM, about 13.5 mM, about 14 mM, about 14.5 mM, about 15 mM, about 15.5 mM, about 16 mM, about 16.5 mM, about 17 mM, about 17.5 mM, about 18 mM, about 18.5 mM, about 19 mM, about 19.5 mM, or about 20 mM propionic acid or a salt thereof (e.g., sodium propionate). In certain embodiments, the first culture medium comprises about 0.5 mM to about 20 mM propionic acid or a salt thereof (e.g., sodium propionate). In certain embodiments, the first culture medium comprises about 5 mM to about 15 mM propionic acid or a salt thereof (e.g., sodium propionate). In certain embodiments, the first culture medium comprises about 5 mM propionic acid or a salt thereof (e.g., sodium propionate). In certain embodiments, the first culture medium comprises about 10 mM propionic acid or a salt thereof (e.g., sodium propionate). In certain embodiments, the first culture medium comprises about 15 mM propionic acid or a salt thereof (e.g., sodium propionate). In certain embodiments, the second culture medium comprises about 0.5 mM to about 20 mM propionic acid or a salt thereof (e.g., sodium propionate). In certain embodiments, the second culture medium comprises about 5 mM to about 15 mM propionic acid or a salt thereof (e.g., sodium propionate). In certain embodiments, the second culture medium comprises about 5 mM propionic acid or a salt thereof (e.g., sodium propionate). In certain embodiments, the second culture medium comprises about 10 mM propionic acid or a salt thereof (e.g., sodium propionate). In certain embodiments, the second culture medium comprises about 15 mM propionic acid or a salt thereof (e.g., sodium propionate).

In certain embodiments, the first culture medium comprises valproic acid or a salt thereof (e.g., sodium valproate) and the second culture medium does not comprise valproic acid or a salt thereof (e.g., sodium valproate). In certain embodiments, the first culture medium does not comprise valproic acid or a salt thereof (e.g., sodium valproate) and the second culture medium comprises valproic acid or a salt thereof (e.g., sodium valproate). In certain embodiments, the first culture medium and second culture medium each comprise up to about 10 mM valproic acid or a salt thereof (e.g., sodium valproate), e.g., no valproic acid or a salt thereof (e.g., sodium valproate), about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 7.5 mM, about 8 mM, about 8.5 mM, about 9 mM, about 9.5 mM, or about 10 mM valproic acid or a salt thereof (e.g., sodium valproate). In certain embodiments, the first culture medium comprises about 0.5 mM to about 10 mM valproic acid or a salt thereof (e.g., sodium valproate). In certain embodiments, the first culture medium comprises about 2.5 mM to about 7.5 mM valproic acid or a salt thereof (e.g., sodium valproate). In certain embodiments, the first culture medium comprises about 2.5 mM valproic acid or a salt thereof (e.g., sodium valproate). In certain embodiments, the first culture medium comprises about 5 mM valproic acid or a salt thereof (e.g., sodium valproate). In certain embodiments, the first culture medium comprises about 7.5 mM valproic acid or a salt thereof (e.g., sodium valproate). In certain embodiments, the second culture medium comprises about 0.5 mM to about 10 mM valproic acid or a salt thereof (e.g., sodium valproate). In certain embodiments, the second culture medium comprises about 2.5 mM to about 7.5 mM valproic acid or a salt thereof (e.g., sodium valproate). In certain embodiments, the second culture medium comprises about 2.5 mM valproic acid or a salt thereof (e.g., sodium valproate). In certain embodiments, the second culture medium comprises about 5 mM valproic acid or a salt thereof (e.g., sodium valproate). In certain embodiments, the second culture medium comprises about 7.5 mM valproic acid or a salt thereof (e.g., sodium valproate).

In certain embodiments, the first culture medium comprises butyric acid or a salt thereof (e.g., sodium butyrate) and the second culture medium does not comprise butyric acid or a salt thereof (e.g., sodium butyrate). In certain embodiments, the first culture medium does not comprise butyric acid or a salt thereof (e.g., sodium butyrate) and the second culture medium comprises butyric acid or a salt thereof (e.g., sodium butyrate). In certain embodiments, the first culture medium and second culture medium each comprise up to about 10 mM butyric acid or a salt thereof (e.g., sodium butyrate), e.g., no butyric acid or a salt thereof (e.g., sodium butyrate), about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 7.5 mM, about 8 mM, about 8.5 mM, about 9 mM, about 9.5 mM, or about 10 mM butyric acid or a salt thereof (e.g., sodium butyrate). In certain embodiments, the first culture medium comprises about 0.5 mM to about 10 mM butyric acid or a salt thereof (e.g., sodium butyrate). In certain embodiments, the first culture medium comprises about 2.5 mM to about 7.5 mM butyric acid or a salt thereof (e.g., sodium butyrate). In certain embodiments, the first culture medium comprises about 2.5 mM butyric acid or a salt thereof (e.g., sodium butyrate). In certain embodiments, the first culture medium comprises about 5 mM butyric acid or a salt thereof (e.g., sodium butyrate). In certain embodiments, the first culture medium comprises about 7.5 mM butyric acid or a salt thereof (e.g., sodium butyrate). In certain embodiments, the second culture medium comprises about 0.5 mM to about 10 mM butyric acid or a salt thereof (e.g., sodium butyrate). In certain embodiments, the second culture medium comprises about 2.5 mM to about 7.5 mM butyric acid or a salt thereof (e.g., sodium butyrate). In certain embodiments, the second culture medium comprises about 2.5 mM butyric acid or a salt thereof (e.g., sodium butyrate). In certain embodiments, the second culture medium comprises about 5 mM butyric acid or a salt thereof (e.g., sodium butyrate). In certain embodiments, the second culture medium comprises about 7.5 mM butyric acid or a salt thereof (e.g., sodium butyrate).

In certain embodiments, the AAV producer cell is cultured in a first culture medium under a first set of conditions, and/or the AAV producer cell is cultured in a second culture medium under a second set of conditions. In certain embodiments, the AAV producer cell is cultured in a first culture medium under a first set of conditions, and the AAV producer cell is cultured in a second culture medium under a second set of conditions. The first and/or second set of conditions may include any parameters that can be varied to at least allow for the minimal growth and/or production (e.g., production of an rAAV particle) of the AAV producer cell. In certain embodiments, the first and/or second set of conditions may include any parameters that can be varied to optimize the growth and/or production efficiency (e.g., production of an rAAV particle) of the AAV producer cell. Parameters include, without limitation, culture temperature, culture period of time, dissolved oxygen level, partial pressure of carbon dioxide (CO₂), culture osmolality, and pH level. In certain embodiments, the AAV producer cell is cultured in a first culture medium at a first temperature, at a first dissolved oxygen level, and/or at a first pH level, for a first period of time; and/or the AAV producer cell is cultured in a second culture medium at a second temperature, at a second dissolved oxygen level, and/or at a second pH level, for a second period of time. In certain embodiments, the AAV producer cell is cultured in a first culture medium at a first temperature, at a first dissolved oxygen level, and/or at a first pH level, for a first period of time; and the AAV producer cell is cultured in a second culture medium at a second temperature, at a second dissolved oxygen level, and/or at a second pH level, for a second period of time.

In certain embodiments, the AAV producer cell is cultured in a first culture medium at a first temperature, and/or the AAV producer cell is cultured in a second culture medium at a second temperature. In certain embodiments, the first and/or second temperature is about 30° C. to about 42° C., e.g., about 30° C., about 30.5° C., about 31° C., about 31.5° C., about 32° C., about 32.5° C., about 33° C., about 33.5° C., about 34° C., about 34.5° C., about 35° C., about 35.5° C., about 36° C., about 36.5° C., about 37° C., about 37.5° C., about 38° C., about 38.5° C., about 39° C., about 39.5° C., about 40° C., about 40.5° C., about 41° C., about 41.5° C., or about 42° C. In certain embodiments, the first and/or second temperature is about 30° C. to about 37° C. In certain embodiments, the first and/or second temperature is about 38° C. to about 42° C. In certain embodiments, the first and/or second temperature is about 37° C. In certain embodiments, the first and/or second temperature is about 39° C. In certain embodiments, the first temperature and the second temperature are the same. In certain embodiments, the first temperature and the second temperature are different. In certain embodiments, the first temperature is about 30° C. to about 37° C., and the second temperature is about 38° C. to about 42° C. In certain embodiments, the first temperature is about 37° C., and the second temperature is about 38° C. to about 42° C. In certain embodiments, the first temperature is about 30° C. to about 37° C., and the second temperature is about 39° C. In certain embodiments, the first temperature is about 37° C., and the second temperature is about 39° C. In certain embodiments, the second temperature is about 30° C. to about 37° C., and the first temperature is about 38° C. to about 42° C. In certain embodiments, the second temperature is about 37° C., and the first temperature is about 38° C. to about 42° C. In certain embodiments, the second temperature is about 30° C. to about 37° C., and the first temperature is about 39° C. In certain embodiments, the second temperature is about 37° C., and the first temperature is about 39° C.

In certain embodiments, the AAV producer cell is cultured in a first culture medium at a first dissolved oxygen level, and/or the AAV producer cell is cultured in a second culture medium at a second dissolved oxygen level. In certain embodiments, the first and/or second dissolved oxygen level is about 1% dissolved oxygen (DO) to about 70% DO, e.g., about 1% DO, about 2% DO, about 3% DO, about 4% DO, about 5% DO, about 6% DO, about 7% DO, about 8% DO, about 9% DO, about 10% DO, about 11% DO, about 12% DO, about 13% DO, about 14% DO, about 15% DO, about 16% DO, about 17% DO, about 18% DO, about 19% DO, about 20% DO, about 21% DO, about 22% DO, about 23% DO, about 24% DO, about 25% DO, about 26% DO, about 27% DO, about 28% DO, about 29% DO, about 30% DO, about 31% DO, about 32% DO, about 33% DO, about 34% DO, about 35% DO, about 40% DO, about 45% DO, about 50% DO, about 55% DO, about 60% DO, about 65% DO, or about 70% DO. In certain embodiments, the first and/or second dissolved oxygen level is about 10% DO to about 70% DO.

In certain embodiments, the AAV producer cell is cultured in a first culture medium at a first pH level, and/or the AAV producer cell is cultured in a second culture medium at a second pH level. In certain embodiments, the pH level of the first and/or second culture medium is about 6.5 to about 7.5, e.g., about 6.5, about 6.6, about 6.7, about 6.8, about 6.9, about 7, about 7.1, about 7.2, about 7.3, about 7.4, or about 7.5. In certain embodiments, the pH level of the first and/or second culture medium is about 6.8 to about 7.2. In certain embodiments, the pH level of the first and/or second culture medium is about 6.8 to about 7. In certain embodiments, the pH level of the first and/or second culture medium is about 6.7. In certain embodiments, the pH level of the first and/or second culture medium is about 6.8. In certain embodiments, the pH level of the first and/or second culture medium is about 7. In certain embodiments, the pH level of the first and/or second culture medium is about 7.2. In certain embodiments, the first pH level and the second pH level are the same. In certain embodiments, the first pH level and the second pH level are different. In certain embodiments, the first pH level is about 7.2 and the second pH level is about 7. In certain embodiments, the first pH level is about 7.2 and the second pH level is about 6.8. In certain embodiments, the first pH level is about 7.2 and the second pH level is about 6.7. In certain embodiments, the second pH level is about 7.2 and the first pH level is about 7. In certain embodiments, the second pH level is about 7.2 and the first pH level is about 6.8. In certain embodiments, the second pH level is about 7.2 and the first pH level is about 6.7.

Any combinations of the aforementioned parameters can be employed in a method described herein. For example, in certain embodiments, the first and/or second temperature is about 30° C. to about 42° C., e.g., about 30° C., about 30.5° C., about 31° C., about 31.5° C., about 32° C., about 32.5° C., about 33° C., about 33.5° C., about 34° C., about 34.5° C., about 35° C., about 35.5° C., about 36° C., about 36.5° C., about 37° C., about 37.5° C., about 38° C., about 38.5° C., about 39° C., about 39.5° C., about 40° C., about 40.5° C., about 41° C., about 41.5° C., or about 42° C.; the first and/or second dissolved oxygen level is about 1% dissolved oxygen (DO) to about 70% DO, e.g., about 1% DO, about 2% DO, about 3% DO, about 4% DO, about 5% DO, about 6% DO, about 7% DO, about 8% DO, about 9% DO, about 10% DO, about 11% DO, about 12% DO, about 13% DO, about 14% DO, about 15% DO, about 16% DO, about 17% DO, about 18% DO, about 19% DO, about 20% DO, about 21% DO, about 22% DO, about 23% DO, about 24% DO, about 25% DO, about 26% DO, about 27% DO, about 28% DO, about 29% DO, about 30% DO, about 31% DO, about 32% DO, about 33% DO, about 34% DO, about 35% DO, about 40% DO, about 45% DO, about 50% DO, about 55% DO, about 60% DO, about 65% DO, or about 70% DO; and/or the pH level of the first and/or second culture medium is about 6.5 to about 7.5, e.g., about 6.8 to about 7, e.g., about 6.5, about 6.6, about 6.7, about 6.8, about 6.9, about 7, about 7.1, about 7.2, about 7.3, about 7.4, or about 7.5.

In certain embodiments, a method for producing rAAV particles described herein comprises culturing the AAV producer cell in a first culture medium at about 30° C. to about 42° C. for a first period of time, wherein the first culture medium has a dissolved oxygen level of about 10% to about 70% dissolved oxygen, and a pH level of about 6.7 to about 7.2. In certain embodiments, a method for producing rAAV particles described herein comprises culturing the AAV producer cell in a first culture medium at about 30° C. to about 42° C. for a first period of time, wherein the first culture medium has a dissolved oxygen level of about 10% to about 70% dissolved oxygen, and a pH level of about 6.7 to about 7.4. In certain embodiments, the method comprises culturing the AAV producer cell in a first culture medium at about 37° C. for a first period of time, wherein the first culture medium has a dissolved oxygen level of about 10% to about 70% dissolved oxygen, and a pH of about 7.2.

In certain embodiments, a method for producing rAAV particles described herein comprises culturing the AAV producer cell in a second culture medium at about 30° C. to about 42° C. for a second period of time, wherein the second culture medium has a dissolved oxygen level of about 10% to about 70% dissolved oxygen, and a pH level of about 6.7 to about 7.2. In certain embodiments, a method for producing rAAV particles described herein comprises culturing the AAV producer cell in a second culture medium at about 30° C. to about 42° C. for a second period of time, wherein the second culture medium has a dissolved oxygen level of about 10% to about 70% dissolved oxygen, and a pH level of about 6.7 to about 7.4. In certain embodiments, the method comprises culturing the AAV producer cell in a second culture medium at about 37° C. for a second period of time, wherein the second culture medium has a dissolved oxygen level of about 10% to about 70% dissolved oxygen, and a pH of about 7.2.

In certain embodiments, the method comprises culturing the AAV producer cell in a first culture medium at about 30° C. to about 42° C. for a first period of time, wherein the first culture medium has a dissolved oxygen level of about 10% to about 70% dissolved oxygen, and a pH level of about 6.7 to about 7.2; and culturing the AAV producer cell in a second culture medium at about 30° C. to about 42° C. for a second period of time, wherein the second culture medium has a dissolved oxygen level of about 10% to about 70% dissolved oxygen, and a pH level of about 6.7 to about 7.2. In certain embodiments, the method comprises culturing the AAV producer cell in a first culture medium at about 30° C. to about 42° C. for a first period of time, wherein the first culture medium has a dissolved oxygen level of about 10% to about 70% dissolved oxygen, and a pH level of about 6.7 to about 7.4; and culturing the AAV producer cell in a second culture medium at about 30° C. to about 42° C. for a second period of time, wherein the second culture medium has a dissolved oxygen level of about 10% to about 70% dissolved oxygen, and a pH level of about 6.7 to about 7.4. In certain embodiments, the method comprises culturing the AAV producer cell in a first culture medium at about 37° C. for a first period of time, wherein the first culture medium has a dissolved oxygen level of about 10% to about 70% dissolved oxygen, and a pH level of about 7.2; and culturing the AAV producer cell in a second culture medium at about 39° C. for a second period of time, wherein the second culture medium has a dissolved oxygen level of about 10% to about 70% dissolved oxygen, and a pH level of about 6.7. In certain embodiments, the method comprises culturing the AAV producer cell in a first culture medium at about 37° C. for a first period of time, wherein the first culture medium has a dissolved oxygen level of about 10% to about 70% dissolved oxygen, and a pH level of about 7.4; and culturing the AAV producer cell in a second culture medium at about 39° C. for a second period of time, wherein the second culture medium has a dissolved oxygen level of about 10% to about 70% dissolved oxygen, and a pH level of about 6.7. In certain embodiments, the method comprises culturing the AAV producer cell in a first culture medium at about 37° C. for a first period of time, wherein the first culture medium has a dissolved oxygen level of about 10% to about 70% dissolved oxygen, and a pH level of about 7.2; and culturing the AAV producer cell in a second culture medium at about 39° C. for a second period of time, wherein the second culture medium has a dissolved oxygen level of about 10% to about 70% dissolved oxygen, and a pH level of about 6.8. In certain embodiments, the method comprises culturing the AAV producer cell in a first culture medium at about 37° C. for a first period of time, wherein the first culture medium has a dissolved oxygen level of about 10% to about 70% dissolved oxygen, and a pH level of about 7.4; and culturing the AAV producer cell in a second culture medium at about 39° C. for a second period of time, wherein the second culture medium has a dissolved oxygen level of about 10% to about 70% dissolved oxygen, and a pH level of about 6.8. In certain embodiments, the method comprises culturing the AAV producer cell in a first culture medium at about 37° C. for a first period of time, wherein the first culture medium has a dissolved oxygen level of about 10% to about 70% dissolved oxygen, and a pH level of about 7.2; and culturing the AAV producer cell in a second culture medium at about 39° C. for a second period of time, wherein the second culture medium has a dissolved oxygen level of about 10% to about 70% dissolved oxygen, and a pH level of about 7. In certain embodiments, the method comprises culturing the AAV producer cell in a first culture medium at about 37° C. for a first period of time, wherein the first culture medium has a dissolved oxygen level of about 10% to about 70% dissolved oxygen, and a pH level of about 7.4; and culturing the AAV producer cell in a second culture medium at about 39° C. for a second period of time, wherein the second culture medium has a dissolved oxygen level of about 10% to about 70% dissolved oxygen, and a pH level of about 7.

In certain embodiments, the method comprises culturing the AAV producer cell in a second culture medium at about 30° C. to about 42° C. for a second period of time, wherein the second culture medium has a dissolved oxygen level of about 10% to about 70% dissolved oxygen, and a pH level of about 6.7 to about 7.2; and culturing the AAV producer cell in a first culture medium at about 30° C. to about 42° C. for a first period of time, wherein the first culture medium has a dissolved oxygen level of about 10% to about 70% dissolved oxygen, and a pH level of about 6.7 to about 7.2. In certain embodiments, the method comprises culturing the AAV producer cell in a second culture medium at about 30° C. to about 42° C. for a second period of time, wherein the second culture medium has a dissolved oxygen level of about 10% to about 70% dissolved oxygen, and a pH level of about 6.7 to about 7.4; and culturing the AAV producer cell in a first culture medium at about 30° C. to about 42° C. for a first period of time, wherein the first culture medium has a dissolved oxygen level of about 10% to about 70% dissolved oxygen, and a pH level of about 6.7 to about 7.4. In certain embodiments, the method comprises culturing the AAV producer cell in a second culture medium at about 37° C. for a second period of time, wherein the second culture medium has a dissolved oxygen level of about 10% to about 70% dissolved oxygen, and a pH level of about 7.2; and culturing the AAV producer cell in a first culture medium at about 39° C. for a first period of time, wherein the first culture medium has a dissolved oxygen level of about 10% to about 70% dissolved oxygen, and a pH level of about 6.7. In certain embodiments, the method comprises culturing the AAV producer cell in a second culture medium at about 37° C. for a second period of time, wherein the second culture medium has a dissolved oxygen level of about 10% to about 70% dissolved oxygen, and a pH level of about 7.4; and culturing the AAV producer cell in a first culture medium at about 39° C. for a first period of time, wherein the first culture medium has a dissolved oxygen level of about 10% to about 70% dissolved oxygen, and a pH level of about 6.7. In certain embodiments, the method comprises culturing the AAV producer cell in a second culture medium at about 37° C. for a second period of time, wherein the second culture medium has a dissolved oxygen level of about 10% to about 70% dissolved oxygen, and a pH level of about 7.2; and culturing the AAV producer cell in a first culture medium at about 39° C. for a first period of time, wherein the first culture medium has a dissolved oxygen level of about 10% to about 70% dissolved oxygen, and a pH level of about 6.8. In certain embodiments, the method comprises culturing the AAV producer cell in a second culture medium at about 37° C. for a second period of time, wherein the second culture medium has a dissolved oxygen level of about 10% to about 70% dissolved oxygen, and a pH level of about 7.4; and culturing the AAV producer cell in a first culture medium at about 39° C. for a first period of time, wherein the first culture medium has a dissolved oxygen level of about 10% to about 70% dissolved oxygen, and a pH level of about 6.8. In certain embodiments, the method comprises culturing the AAV producer cell in a second culture medium at about 37° C. for a second period of time, wherein the second culture medium has a dissolved oxygen level of about 10% to about 70% dissolved oxygen, and a pH level of about 7.2; and culturing the AAV producer cell in a first culture medium at about 39° C. for a first period of time, wherein the first culture medium has a dissolved oxygen level of about 10% to about 70% dissolved oxygen, and a pH level of about 7. In certain embodiments, the method comprises culturing the AAV producer cell in a second culture medium at about 37° C. for a second period of time, wherein the second culture medium has a dissolved oxygen level of about 10% to about 70% dissolved oxygen, and a pH level of about 7.4; and culturing the AAV producer cell in a first culture medium at about 39° C. for a first period of time, wherein the first culture medium has a dissolved oxygen level of about 10% to about 70% dissolved oxygen, and a pH level of about 7. In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for a first period of time; and (c) culturing the AAV producer cell in a second culture medium at a second temperature for a second period of time, wherein the second culture medium comprises about 0.1% (v/v) to about 5% (v/v) dimethyl sulfoxide (DMSO) and/or wherein the second temperature is about 38° C. to about 42° C., such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for a first period of time, wherein the first culture medium comprises about 0.1% (v/v) to about 5% (v/v) dimethyl sulfoxide (DMSO) and/or wherein the first temperature is about 38° C. to about 42° C.; and (c) culturing the AAV producer cell in a second culture medium at a second temperature for a second period of time, such that rAAV particles are produced by the AAV producer cell.

In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for a first period of time, wherein the first culture medium does not comprise DMSO and the first temperature is about 30° C. to about 37° C.; and (c) culturing the AAV producer cell in a second culture medium at a second temperature for a second period of time, wherein the second culture medium comprises about 0.1% (v/v) to about 5% (v/v) dimethyl sulfoxide (DMSO) and/or wherein the second temperature is about 38° C. to about 42° C., such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a second culture medium at a second temperature for a second period of time, wherein the second culture medium does not comprise DMSO and the second temperature is about 30° C. to about 37° C.; and (c) culturing the AAV producer cell in a first culture medium at a first temperature for a first period of time, wherein the first culture medium comprises about 0.1% (v/v) to about 5% (v/v) dimethyl sulfoxide (DMSO) and/or wherein the first temperature is about 38° C. to about 42° C., such that rAAV particles are produced by the AAV producer cell.

In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for a first period of time, wherein the first culture medium does not comprise DMSO and the first temperature is about 37° C.; and (c) culturing the AAV producer cell in a second culture medium at a second temperature for a second period of time, wherein the second culture medium comprises about 1.5% (v/v) dimethyl sulfoxide (DMSO) and/or wherein the second temperature is about 39° C., such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a second culture medium at a second temperature for a second period of time, wherein the second culture medium does not comprise DMSO and the second temperature is about 37° C.; and (c) culturing the AAV producer cell in a first culture medium at a first temperature for a first period of time, wherein the first culture medium comprises about 1.5% (v/v) dimethyl sulfoxide (DMSO) and/or wherein the first temperature is about 39° C., such that rAAV particles are produced by the AAV producer cell.

In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for a first period of time, wherein the first culture medium does not comprise DMSO and the first temperature is about 37° C.; and (c) culturing the AAV producer cell in a second culture medium at a second temperature for a second period of time, wherein the second culture medium comprises about 1.5% (v/v) dimethyl sulfoxide (DMSO) and wherein the second temperature is about 39° C., such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a second culture medium at a second temperature for a second period of time, wherein the second culture medium does not comprise DMSO and the second temperature is about 37° C.; and (c) culturing the AAV producer cell in a first culture medium at a first temperature for a first period of time, wherein the first culture medium comprises about 1.5% (v/v) dimethyl sulfoxide (DMSO) and wherein the first temperature is about 39° C., such that rAAV particles are produced by the AAV producer cell.

In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for a first period of time, wherein the first culture medium has a pH of about 7.2, does not comprise DMSO, and the first temperature is about 37° C.; and (c) culturing the AAV producer cell in a second culture medium at a second temperature for a second period of time, wherein the second culture medium has a pH of about 6.7 or 6.8, comprises about 1.5% (v/v) dimethyl sulfoxide (DMSO), and the second temperature is about 39° C., such that rAAV particles are produced by the AAV producer cell.

In the various methods described herein, a first period of time, e.g., in the context of culturing the AAV producer cell for a first period of time, is from 0 to about 5 hours. In certain embodiments, the first period of time is 0 hours, about 0.1 hours, about 0.2 hours, about 0.3 hours, about 0.4 hours, about 0.5 hours, about 0.6 hours, about 0.7 hours, about 0.8 hours, about 0.9 hours, about 1 hour, about 1.1 hours, about 1.2 hours, about 1.3 hours, about 1.4 hours, about 1.5 hours, about 1.6 hours, about 1.7 hours, about 1.8 hours, about 1.9 hours, about 2 hours, about 2.1 hours, about 2.2 hours, about 2.3 hours, about 2.4 hours, about 2.5 hours, about 2.6 hours, about 2.7 hours, about 2.8 hours, about 2.9 hours, about 3 hours, about 3.1 hours, about 3.2 hours, about 3.3 hours, about 3.4 hours, about 3.5 hours, about 3.6 hours, about 3.7 hours, about 3.8 hours, about 3.9 hours, about 4 hours, about 4.1 hours, about 4.2 hours, about 4.3 hours, about 4.4 hours, about 4.5 hours, about 4.6 hours, about 4.7 hours, about 4.8 hours, about 4.9 hours, or about 5 hours. In certain embodiments, the first period of time is about 2 hours.

In the various methods described herein, a second period of time, e.g., in the context of culturing the AAV producer cell for a second period of time, is about 1 to about 100 hours. In certain embodiments, the second period of time is about 1 hour, about 5 hours, about 10 hours, about 15 hours, about 20 hours, about 25 hours, about 30 hours, about 35 hours, about 40 hours, about 45 hours, about 48 hours, about 50 hours, about 55 hours, about 60 hours, about 65 hours, about 70 hours, about 75 hours, about 80 hours, about 85 hours, about 90 hours, about 95 hours, or about 100 hours. In certain embodiments, the second period of time is about 48 to about 75 hours. In certain embodiments, the second period of time is about 65 hours to about 75 hours. In certain embodiments, the second period of time is about 70 hours.

Accordingly, in certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for a first period of time, wherein the first period of time is 0 to about 5 hours (e.g., about 0.5 hours, about 1 hour, about 1.5 hours, about 2 hours, about 2.5 hours, about 3 hours, about 3.5 hours, about 4 hours, about 4.5 hours, or about 5 hours); and (c) culturing the AAV producer cell in a second culture medium at a second temperature for a second period of time, wherein the second period of time is about 1 to about 100 hours, such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for a first period of time, wherein the first period of time is about 0.5 hours; and (c) culturing the AAV producer cell in a second culture medium at a second temperature for a second period of time, wherein the second period of time is about 48 to about 75 hours, optionally about 65 to about 75 hours, optionally 72 hours, such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for a first period of time, wherein the first period of time is about 1 hour; and (c) culturing the AAV producer cell in a second culture medium at a second temperature for a second period of time, wherein the second period of time is about 48 to about 75 hours, optionally about 65 to about 75 hours, optionally 72 hours, such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for a first period of time, wherein the first period of time is about 1.5 hours; and (c) culturing the AAV producer cell in a second culture medium at a second temperature for a second period of time, wherein the second period of time is about 48 to about 75 hours, optionally about 65 to about 75 hours, optionally 72 hours, such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for a first period of time, wherein the first period of time is about 2 hours; and (c) culturing the AAV producer cell in a second culture medium at a second temperature for a second period of time, wherein the second period of time is about 48 to about 75 hours, optionally about 65 to about 75 hours, optionally 72 hours, such that rAAV particles are produced by the AAV producer cell.

Accordingly, in certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for 0 to about 5 hours (e.g., about 0.5 hours, about 1 hour, about 1.5 hours, about 2 hours, about 2.5 hours, about 3 hours, about 3.5 hours, about 4 hours, about 4.5 hours, or about 5 hours), wherein the first culture medium does not comprise DMSO and the first temperature is about 37° C.; and (c) culturing the AAV producer cell in a second culture medium at a second temperature for about 1 to about 100 hours, wherein the second culture medium comprises about 1.5% (v/v) dimethyl sulfoxide (DMSO) and wherein the second temperature is about 39° C., such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for about 0.5 hours, wherein the first culture medium does not comprise DMSO and the first temperature is about 37° C.; and (c) culturing the AAV producer cell in a second culture medium at a second temperature for about 48 to about 75 hours, optionally about 65 to about 75 hours, optionally 72 hours, wherein the second culture medium comprises about 1.5% (v/v) dimethyl sulfoxide (DMSO) and wherein the second temperature is about 39° C., such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for about 1 hour, wherein the first culture medium does not comprise DMSO and the first temperature is about 37° C.; and (c) culturing the AAV producer cell in a second culture medium at a second temperature for about 48 to about 75 hours, optionally about 65 to about 75 hours, optionally 72 hours, wherein the second culture medium comprises about 1.5% (v/v) dimethyl sulfoxide (DMSO) and wherein the second temperature is about 39° C., such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for about 1.5 hours, wherein the first culture medium does not comprise DMSO and the first temperature is about 37° C.; and (c) culturing the AAV producer cell in a second culture medium at a second temperature for about 48 to about 75 hours, optionally about 65 to about 75 hours, optionally 72 hours, wherein the second culture medium comprises about 1.5% (v/v) dimethyl sulfoxide (DMSO) and wherein the second temperature is about 39° C., such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for about 2 hours, wherein the first culture medium does not comprise DMSO and the first temperature is about 37° C.; and (c) culturing the AAV producer cell in a second culture medium at a second temperature for about 48 to about 75 hours, optionally about 65 to about 75 hours, optionally 72 hours, wherein the second culture medium comprises about 1.5% (v/v) dimethyl sulfoxide (DMSO) and wherein the second temperature is about 39° C., such that rAAV particles are produced by the AAV producer cell.

In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for 0 to about 5 hours (e.g., about 0.5 hours, about 1 hour, about 1.5 hours, about 2 hours, about 2.5 hours, about 3 hours, about 3.5 hours, about 4 hours, about 4.5 hours, or about 5 hours), wherein the first culture medium has a pH of about 7.2, does not comprise DMSO, and the first temperature is about 37° C.; and (c) culturing the AAV producer cell in a second culture medium at a second temperature for about 1 to about 100 hours, wherein the second culture medium has a pH of about 6.7 or 6.8, comprises about 1.5% (v/v) dimethyl sulfoxide (DMSO), and the second temperature is about 39° C., such that rAAV particles are produced by the AAV producer cell.

In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for about 0.5 hours, wherein the first culture medium has a pH of about 7.2, does not comprise DMSO, and the first temperature is about 37° C.; and (c) culturing the AAV producer cell in a second culture medium at a second temperature for about 48 to about 75 hours, optionally about 65 to about 75 hours, optionally 70 hours, wherein the second culture medium has a pH of about 6.7 to about 7, comprises about 1.5% (v/v) dimethyl sulfoxide (DMSO), comprises a dissolved oxygen level of about 10%, and the second temperature is about 39° C., such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for about 0.5 hours, wherein the first culture medium has a pH of about 7.2, does not comprise DMSO, and the first temperature is about 37° C.; and (c) culturing the AAV producer cell in a second culture medium at a second temperature for about 48 to about 75 hours, optionally about 65 to about 75 hours, optionally 70 hours, wherein the second culture medium has a pH of about 6.7, comprises about 1.5% (v/v) dimethyl sulfoxide (DMSO), comprises a dissolved oxygen level of about 10%, and the second temperature is about 39° C., such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for about 0.5 hours, wherein the first culture medium has a pH of about 7.2, does not comprise DMSO, and the first temperature is about 37° C.; and (c) culturing the AAV producer cell in a second culture medium at a second temperature for about 48 to about 75 hours, optionally about 65 to about 75 hours, optionally 70 hours, wherein the second culture medium has a pH of about 6.8, comprises about 1.5% (v/v) dimethyl sulfoxide (DMSO), comprises a dissolved oxygen level of about 10%, and the second temperature is about 39° C., such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for about 0.5 hours, wherein the first culture medium has a pH of about 7.2, does not comprise DMSO, and the first temperature is about 37° C.; and (c) culturing the AAV producer cell in a second culture medium at a second temperature for about 48 to about 75 hours, optionally about 65 to about 75 hours, optionally 70 hours, wherein the second culture medium has a pH of about 7, comprises about 1.5% (v/v) dimethyl sulfoxide (DMSO), comprises a dissolved oxygen level of about 10%, and the second temperature is about 39° C., such that rAAV particles are produced by the AAV producer cell.

In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for about 1 hour, wherein the first culture medium has a pH of about 7.2, does not comprise DMSO, and the first temperature is about 37° C.; and (c) culturing the AAV producer cell in a second culture medium at a second temperature for about 48 to about 75 hours, optionally about 65 to about 75 hours, optionally 70 hours, wherein the second culture medium has a pH of about 6.7 to about 7, comprises about 1.5% (v/v) dimethyl sulfoxide (DMSO), comprises a dissolved oxygen level of about 10%, and the second temperature is about 39° C., such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for about 1 hour, wherein the first culture medium has a pH of about 7.2, does not comprise DMSO, and the first temperature is about 37° C.; and (c) culturing the AAV producer cell in a second culture medium at a second temperature for about 48 to about 75 hours, optionally about 65 to about 75 hours, optionally 70 hours, wherein the second culture medium has a pH of about 6.7, comprises about 1.5% (v/v) dimethyl sulfoxide (DMSO), comprises a dissolved oxygen level of about 10%, and the second temperature is about 39° C., such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for about 1 hour, wherein the first culture medium has a pH of about 7.2, does not comprise DMSO, and the first temperature is about 37° C.; and (c) culturing the AAV producer cell in a second culture medium at a second temperature for about 48 to about 75 hours, optionally about 65 to about 75 hours, optionally 70 hours, wherein the second culture medium has a pH of about 6.8, comprises about 1.5% (v/v) dimethyl sulfoxide (DMSO), comprises a dissolved oxygen level of about 10%, and the second temperature is about 39° C., such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for about 1 hour, wherein the first culture medium has a pH of about 7.2, does not comprise DMSO, and the first temperature is about 37° C.; and (c) culturing the AAV producer cell in a second culture medium at a second temperature for about 48 to about 75 hours, optionally about 65 to about 75 hours, optionally 70 hours, wherein the second culture medium has a pH of about 7, comprises about 1.5% (v/v) dimethyl sulfoxide (DMSO), comprises a dissolved oxygen level of about 10%, and the second temperature is about 39° C., such that rAAV particles are produced by the AAV producer cell.

In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for about 1.5 hours, wherein the first culture medium has a pH of about 7.2, does not comprise DMSO, and the first temperature is about 37° C.; and (c) culturing the AAV producer cell in a second culture medium at a second temperature for about 48 to about 75 hours, optionally about 65 to about 75 hours, optionally 70 hours, wherein the second culture medium has a pH of about 6.7 to about 7, comprises about 1.5% (v/v) dimethyl sulfoxide (DMSO), comprises a dissolved oxygen level of about 10%, and the second temperature is about 39° C., such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for about 1.5 hours, wherein the first culture medium has a pH of about 7.2, does not comprise DMSO, and the first temperature is about 37° C.; and (c) culturing the AAV producer cell in a second culture medium at a second temperature for about 48 to about 75 hours, optionally about 65 to about 75 hours, optionally 70 hours, wherein the second culture medium has a pH of about 6.7, comprises about 1.5% (v/v) dimethyl sulfoxide (DMSO), comprises a dissolved oxygen level of about 10%, and the second temperature is about 39° C., such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for about 1.5 hours, wherein the first culture medium has a pH of about 7.2, does not comprise DMSO, and the first temperature is about 37° C.; and (c) culturing the AAV producer cell in a second culture medium at a second temperature for about 48 to about 75 hours, optionally about 65 to about 75 hours, optionally 70 hours, wherein the second culture medium has a pH of about 6.8, comprises about 1.5% (v/v) dimethyl sulfoxide (DMSO), comprises a dissolved oxygen level of about 10%, and the second temperature is about 39° C., such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for about 1.5 hours, wherein the first culture medium has a pH of about 7.2, does not comprise DMSO, and the first temperature is about 37° C.; and (c) culturing the AAV producer cell in a second culture medium at a second temperature for about 48 to about 75 hours, optionally about 65 to about 75 hours, optionally 70 hours, wherein the second culture medium has a pH of about 7, comprises about 1.5% (v/v) dimethyl sulfoxide (DMSO), comprises a dissolved oxygen level of about 10%, and the second temperature is about 39° C., such that rAAV particles are produced by the AAV producer cell.

In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for about 2 hours, wherein the first culture medium has a pH of about 7.2, does not comprise DMSO, and the first temperature is about 37° C.; and (c) culturing the AAV producer cell in a second culture medium at a second temperature for about 48 to about 75 hours, optionally about 65 to about 75 hours, optionally 70 hours, wherein the second culture medium has a pH of about 6.7 to about 7, comprises about 1.5% (v/v) dimethyl sulfoxide (DMSO), comprises a dissolved oxygen level of about 10%, and the second temperature is about 39° C., such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for about 2 hours, wherein the first culture medium has a pH of about 7.2, does not comprise DMSO, and the first temperature is about 37° C.; and (c) culturing the AAV producer cell in a second culture medium at a second temperature for about 48 to about 75 hours, optionally about 65 to about 75 hours, optionally 70 hours, wherein the second culture medium has a pH of about 6.7, comprises about 1.5% (v/v) dimethyl sulfoxide (DMSO), comprises a dissolved oxygen level of about 10%, and the second temperature is about 39° C., such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for about 2 hours, wherein the first culture medium has a pH of about 7.2, does not comprise DMSO, and the first temperature is about 37° C.; and (c) culturing the AAV producer cell in a second culture medium at a second temperature for about 48 to about 75 hours, optionally about 65 to about 75 hours, optionally 70 hours, wherein the second culture medium has a pH of about 6.8, comprises about 1.5% (v/v) dimethyl sulfoxide (DMSO), comprises a dissolved oxygen level of about 10%, and the second temperature is about 39° C., such that rAAV particles are produced by the AAV producer cell. In certain embodiments, a method for producing rAAV particles comprises: (a) introducing into a mammalian cell a first polynucleotide comprising an rAAV genome, to generate an AAV producer cell; (b) culturing the AAV producer cell in a first culture medium at a first temperature for about 2 hours, wherein the first culture medium has a pH of about 7.2, does not comprise DMSO, and the first temperature is about 37° C.; and (c) culturing the AAV producer cell in a second culture medium at a second temperature for about 48 to about 75 hours, optionally about 65 to about 75 hours, optionally 70 hours, wherein the second culture medium has a pH of about 7, comprises about 1.5% (v/v) dimethyl sulfoxide (DMSO), comprises a dissolved oxygen level of about 10%, and the second temperature is about 39° C., such that rAAV particles are produced by the AAV producer cell.

In certain embodiments, prior to introduction of the first polynucleotide into the mammalian cell, the mammalian cell is cultured in a third culture medium at a third temperature for a third period of time. In certain embodiments, the mammalian cell is cultured in a third culture medium at a third temperature for a third period of time prior to the introduction of the first polynucleotide into the mammalian cell. In certain embodiments, the mammalian cell is cultured in a third culture medium under a third set of conditions, for a third period of time. The third culture medium can comprise any of the aforementioned culture media and/or additives. The third set of conditions may include any of the aforementioned parameters.

In certain embodiments, the third culture medium comprises an additive selected from the group consisting of dimethyl sulfoxide (DMSO), valproic acid, propionic acid or a salt thereof, and butyric acid or a salt thereof.

In certain embodiments, the third culture medium up to about 5% (v/v) DMSO, e.g., no DMSO, about 0.1% (v/v), about 0.2% (v/v), about 0.3% (v/v), about 0.4% (v/v), about 0.5% (v/v), about 0.6% (v/v), about 0.7% (v/v), about 0.8% (v/v), about 0.9% (v/v), about 1% (v/v), about 1.1% (v/v), about 1.2% (v/v), about 1.3% (v/v), about 1.4% (v/v), about 1.5% (v/v), about 1.6% (v/v), about 1.7% (v/v), about 1.8% (v/v), about 1.9% (v/v), about 2% (v/v), about 2.1% (v/v), about 2.2% (v/v), about 2.30% (v/v), about 2.4% (v/v), about 2.5% (v/v), about 2.6% (v/v), about 2.7% (v/v), about 2.8% (v/v), about 2.9% (v/v), about 3% (v/v), about 3.1% (v/v), about 3.2% (v/v), about 3.3% (v/v), about 3.4% (v/v), about 3.5% (v/v), about 3.6% (v/v), about 3.7% (v/v), about 3.8% (v/v), about 3.9% (v/v), about 4% (v/v), about 4.1% (v/v), about 4.2% (v/v), about 4.3% (v/v), about 4.4% (v/v), about 4.5% (v/v), about 4.6% (v/v), about 4.7% (v/v), about 4.8% (v/v), about 4.9% (v/v), or about 5% (v/v) DMSO. In certain embodiments, the first third medium comprises about 0.1% (v/v) to about 5% (v/v) DMSO. In certain embodiments, the third culture medium comprises about 0.5% (v/v) to about 5% (v/v) DMSO. In certain embodiments, the third culture medium comprises about 1.5% (v/v) DMSO.

In certain embodiments, the third temperature is about 30° C. to about 42° C., e.g., about 30° C., about 30.5° C., about 31° C., about 31.5° C., about 32° C., about 32.5° C., about 33° C., about 33.5° C., about 34° C., about 34.5° C., about 35° C., about 35.5° C., about 36° C., about 36.5° C., about 37° C., about 37.5° C., about 38° C., about 38.5° C., about 39° C., about 39.5° C., about 40° C., about 40.5° C., about 41° C., about 41.5° C., or about 42° C. In certain embodiments, the third temperature is about 30° C. to about 37° C. In certain embodiments, the third temperature is about 37° C.

In certain embodiments, the third period of time is about 0.5 to about 3 hours, e.g., about 0.5 hours, about 1 hours, about 1.5 hours, about 2 hours, about 2.5 hours, or about 3 hours. In certain embodiments, the third period of time is about 2 hours.

Accordingly, in certain embodiments, a method for producing rAAV particles comprises: (a) culturing a mammalian cell in a third culture medium comprising about 0.1% (v/v) to about 5% (v/v) DMSO, optionally 1.5% (v/v) DMSO, at a temperature of about 30° C. to about 37° C., optionally 37° C., for about 0.5 hours to about 3 hours, optionally 2 hours, wherein the third culture medium has a pH of about 7.2; (b) introducing into the mammalian cell a first polynucleotide comprising an rAAV genome, to generate an AAV producer cell; (c) culturing the AAV producer cell in a first culture medium comprising about 0.1% (v/v) to about 5% (v/v) DMSO, optionally 1.5% (v/v) DMSO, at a temperature of about 30° C. to about 37° C., optionally 37° C., for about 0.5 hours to about 5 hours, optionally 2 hours, wherein the first culture medium has a pH of about 7.2; and (d) culturing the AAV producer cell in a second culture medium at about 38° C. to about 42° C., optionally 39° C., for about 48 to about 75 hours, optionally about 65 to about 75 hours, optionally 70 hours, wherein the second culture medium has a pH of about 6.7 to about 7, such that rAAV particles are produced by the AAV producer cell. In certain embodiments, a method for producing rAAV particles comprises: (a) culturing a mammalian cell in a third culture medium comprising about 0.1% (v/v) to about 5% (v/v) DMSO, optionally 1.5% (v/v) DMSO, at a temperature of about 30° C. to about 37° C., optionally 37° C., for about 0.5 hours to about 3 hours, optionally 2 hours, wherein the third culture medium has a pH of about 7.2; (b) introducing into the mammalian cell a first polynucleotide comprising an rAAV genome, to generate an AAV producer cell; (c) culturing the AAV producer cell in a first culture medium comprising about 0.1% (v/v) to about 5% (v/v) DMSO, optionally 1.5% (v/v) DMSO, at a temperature of about 30° C. to about 37° C., optionally 37° C., for about 0.5 hours to about 5 hours, optionally 2 hours, wherein the first culture medium has a pH of about 7.2; and (d) culturing the AAV producer cell in a second culture medium at about 38° C. to about 42° C., optionally 39° C., for about 48 to about 75 hours, optionally about 65 to about 75 hours, optionally 70 hours, wherein the second culture medium has a pH of about 6.7, such that rAAV particles are produced by the AAV producer cell. In certain embodiments, a method for producing rAAV particles comprises: (a) culturing a mammalian cell in a third culture medium comprising about 0.1% (v/v) to about 5% (v/v) DMSO, optionally 1.5% (v/v) DMSO, at a temperature of about 30° C. to about 37° C., optionally 37° C., for about 0.5 hours to about 3 hours, optionally 2 hours, wherein the third culture medium has a pH of about 7.2; (b) introducing into the mammalian cell a first polynucleotide comprising an rAAV genome, to generate an AAV producer cell; (c) culturing the AAV producer cell in a first culture medium comprising about 0.1% (v/v) to about 5% (v/v) DMSO, optionally 1.5% (v/v) DMSO, at a temperature of about 30° C. to about 37° C., optionally 37° C., for about 0.5 hours to about 5 hours, optionally 2 hours, wherein the first culture medium has a pH of about 7.2; and (d) culturing the AAV producer cell in a second culture medium at about 38° C. to about 42° C., optionally 39° C., for about 48 to about 75 hours, optionally about 65 to about 75 hours, optionally 70 hours, wherein the second culture medium has a pH of about 6.8, such that rAAV particles are produced by the AAV producer cell. In certain embodiments, a method for producing rAAV particles comprises: (a) culturing a mammalian cell in a third culture medium comprising about 0.1% (v/v) to about 5% (v/v) DMSO, optionally 1.5% (v/v) DMSO, at a temperature of about 30° C. to about 37° C., optionally 37° C., for about 0.5 hours to about 3 hours, optionally 2 hours, wherein the third culture medium has a pH of about 7.2; (b) introducing into the mammalian cell a first polynucleotide comprising an rAAV genome, to generate an AAV producer cell; (c) culturing the AAV producer cell in a first culture medium comprising about 0.1% (v/v) to about 5% (v/v) DMSO, optionally 1.5% (v/v) DMSO, at a temperature of about 30° C. to about 37° C., optionally 37° C., for about 0.5 hours to about 5 hours, optionally 2 hours, wherein the first culture medium has a pH of about 7.2; and (d) culturing the AAV producer cell in a second culture medium at about 38° C. to about 42° C., optionally 39° C., for about 48 to about 75 hours, optionally about 65 to about 75 hours, optionally 70 hours, wherein the second culture medium has a pH of about 7, such that rAAV particles are produced by the AAV producer cell.

Methods of the present disclosure result in improved productivity, e.g., improved titers of rAAV particles. Various methods of determining the titer of AAV particles in a composition, for example, vector genome (VG) titers can be quantified using droplet digital PCR (ddPCR), and capsid titers can be quantified using an enzyme-linked immunosorbent assay utilizing an antibody specific for a conformational epitope on assembled AAV capsids (capsid ELISA). While ddPCR and capsid ELISA provide valuable information regarding the number of AAV vector genomes and intact AAV particles in a composition, other suitable methods are known in the art.

In certain embodiments, the methods described herein result in improved production efficiency, resulting in increased vector genome titer. In certain embodiments, the methods result in about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195%, about 200%, about 205%, about 210%, about 215%, about 220%, about 225%, about 230%, about 235%, about 240%, about 245%, or about 250% or more increase in vector genome titer.

In certain embodiments, the methods described herein result in improved production efficiency, resulting in increased capsid titer. In certain embodiments, the methods result in about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195%, about 200%, about 205%, about 210%, about 215%, about 220%, about 225%, about 230%, about 235%, about 240%, about 245%, or about 250% or more increase in capsid titer.

In certain embodiments, the methods described herein further comprise purifying and formulating the rAAV particles for administration to a human subject.

### Nucleic Acids, Vectors, and Cells

In one aspect, the present disclosure provides a method for producing recombinant AAV (rAAV) particles, comprising: (a) introducing into a mammalian cell a first polynucleotide comprising an rAAV genome, to generate an AAV producer cell; (b) culturing the AAV producer cell in a first culture medium at a first temperature for a first period of time; and (c) culturing the AAV producer cell in a second culture medium at a second temperature for a second period of time, such that rAAV particles are produced by the AAV producer cell.

Methods described herein for producing rAAV particles comprise introducing into a mammalian cell a first polynucleotide comprising an rAAV genome, to generate an AAV producer cell. In certain embodiments, the method further comprises introducing into the mammalian cell a second polypeptide encoding an AAV capsid protein, a third polynucleotide encoding an AAV Rep protein, and/or a fourth polynucleotide encoding one or more helper virus genes. In certain embodiments, the method further comprises introducing into the mammalian cell a second polynucleotide encoding an AAV capsid protein, a third polynucleotide encoding an AAV Rep protein, and a fourth polynucleotide encoding one or more helper genes.

The first, second, third, and/or fourth polynucleotide can be comprised within a nucleic acid vector. In certain embodiments, the first and second polynucleotide are comprised within the same nucleic acid vector. In certain embodiments, the first, second, and third polynucleotide are comprised within the same nucleic acid vector. In certain embodiments, the first, second, third, and fourth polynucleotide are comprised within the same nucleic acid vector. In certain embodiments, the second, third, and fourth polynucleotide are comprised within the same nucleic acid vector.

Suitable nucleic acid vectors, include, without limitation, plasmids, minimal vectors (e.g., minicircles, Nanoplasmids™, doggybones, MIDGE vectors, and the like), viruses, cosmids, artificial chromosomes, linear DNA, and mRNA. In certain embodiments, the nucleic acid vector is a DNA plasmid or a DNA minimal vector. Any DNA plasmid or DNA minimal vector that can accommodate the necessary vector elements can be used for the first nucleic acid vector and the second nucleic acid vector. Suitable DNA minimal vectors include, without limitation, linear covalently closed DNA (e.g., ministring DNA), linear covalently closed dumbbell shaped DNA (e.g., doggybone DNA, dumbbell DNA), minicircles, Nanoplasmids™ minimalistic immunologically defined gene expression (MIDGE) vectors, and others known to those of skill in the art. DNA minimal vectors and their methods of production are described in, e.g., U.S. Patent Application Nos. 20100233814, 20120282283, 20130216562, 20150218565, 20150218586, 20160008488, 20160215296, 20160355827, 20190185924, 20200277624, and 20210010021, all of which are herein incorporated by reference in their entireties.

In certain embodiments, the polynucleotides in the nucleic acid vectors are optimized, e.g., by codon/RNA optimization, replacement with heterologous signal sequences, and/or elimination of mRNA instability elements. Methods to generate optimized polynucleotides for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly, all of which are herein incorporated by reference in their entireties. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In certain embodiments, it can be desirable to alter one or more codons to encode a conservative mutation, e.g., a similar amino acid with similar chemical structure and properties and/or function as the original amino acid. Such methods can increase expression of the encoded protein (e.g., capsid protein) relative to the expression of the protein (e.g., capsid protein) encoded by polynucleotides that have not been optimized.

In certain embodiments, the helper virus is selected from the group consisting of adenovirus, herpes virus (including herpes simplex virus (HSV)), poxvirus (such as vaccinia virus), cytomegalovirus (CMV), and baculovirus. In certain embodiments, where the helper virus is adenovirus, the adenovirus genome comprises one or more adenovirus RNA genes selected from the group consisting of E1, E2, E4, and VA. In certain embodiments, where the adenovirus genome comprises one or more adenovirus RNA genes selected from the group consisting of E2, E4, and VA. In certain embodiments, where the helper virus is HSV, the HSV genome comprises one or more of HSV genes selected from the group consisting of UL5/8/52, ICPO, ICP4, ICP22, and UL30/UL42.

Suitable mammalian cells include but are not limited to CHO, VERO, BHK, Hela, MDCK, HEK293, HEK293T, HEK293F, HEK293EBNA, NIH 3T3, W138, BT483, Hs578T, HTB2, BT20 and T47D, NS0 (a murine myeloma cell line that does not endogenously produce any immunoglobulin chains), CAP, CAP-T, CRL7030, COS (e.g., COS1 or COS), PER.C6, VERO, AGE1.CR, A549, HsS78Bst, HepG2, C139, EB66, SP210, R1.1, B-W, L-M, BSC1, BSC40, YB/20, BMT10, HsS78Bst cells, and any derivatives thereof. In certain embodiments, the mammalian cell is a mammalian cell that can be grown in a suspension culture. In certain embodiments, the mammalian cell is an HEK293 cell or an HEK293T cell that can be grown in suspension culture.

In certain embodiments, a mammalian cell can be transformed with a polynucleotide (e.g., DNA or RNA) controlled by appropriate transcriptional regulatory elements (e.g., promoter, enhancer, sequences, transcription terminators, polyadenylation sites, etc.), and a selectable marker. Following the introduction of polynucleotide, engineered cells can be allowed to grow for 1-2 days in an enriched media, and then are switched to a selective media. The selectable marker confers resistance to the selection and allows the engineered cells to stably integrate the plasmid into their chromosomes and grow to form foci which in turn can be cloned and expanded into stable cell lines.

A number of selection systems can be used, including but not limited to the herpes simplex virus thymidine kinase (Wigler M et al. (1977) Cell 11(1): 223-32), hypoxanthineguanine phosphoribosyltransferase (Szybalska E H & Szybalski W (1962) PNAS 48(12): 2026-2034), and adenine phosphoribosyltransferase (Lowy I et al. (1980) Cell 22(3): 817-23) genes in tk-, hgprt- or aprt-cells, respectively, all of which are herein incorporated by reference in their entireties. Also, antimetabolite resistance can be used as the basis of selection for the following genes: dhfr, which confers resistance to methotrexate (Wigler M et al. (1980) PNAS 77(6): 3567-70; O'Hare K et al. (1981) PNAS 78: 1527-31); gpt, which confers resistance to mycophenolic acid (Mulligan R C & Berg P (1981) PNAS 78(4): 2072-6); neo, which confers resistance to the aminoglycoside G-418 (Wu G Y & Wu C H (1991) Biotherapy 3: 87-95; Tolstoshev P (1993) Ann Rev Pharmacol Toxicol 32: 573-596; Mulligan R C (1993) Science 260: 926-932; Morgan R A & Anderson W F (1993) Ann Rev Biochem 62: 191-217; Nabel G J & Felgner PL (1993) Trends Biotechnol 11(5): 211-5); and hygro, which confers resistance to hygromycin (Santerre R F et al. (1984) Gene 30(1-3): 147-56), all of which are herein incorporated by reference in their entireties. Methods commonly known in the art of recombinant DNA technology can be routinely applied to select the desired recombinant clone and such methods are described, for example, in Ausubel F M et al. (eds.), Current Protocols in Molecular Biology, John Wiley & Sons, N Y (1993); Kriegler M, Gene Transfer and Expression, A Laboratory Manual, Stockton Press, N Y (1990); and in Chapters 12 and 13, Dracopoli N C et al. (eds.), Current Protocols in Human Genetics, John Wiley & Sons, N Y (1994); Colbere-Garapin F et al. (1981) J Mol Biol 150: 1-14, all of which are herein incorporated by reference in their entireties.

The introduction of nucleic acids (e.g., a first polynucleotide) into a mammalian cell can be achieved by any method known to those of skill in the art. Methods of introducing a nucleic acid into a mammalian cell include, without limitation, mechanical procedures such as liposomal insertion of a plasmid (e.g., lipofection), electroporation, microinjection, or via the use of a viral vector. In certain embodiments, the first culture medium comprises one or more reagents useful for mediating the introduction of a nucleic acid into a cell.

Reagents useful for mediating the introduction of a nucleic acid into a mammalian cell are well known to those of skill in the art. Such reagents include, without limitation, those disclosed in e.g., PCT Publication No. WO 2013/166339A1, one or more compounds and/or compositions comprising cationic polymers such as polyethyleneimine (PEI), polymers of positively charged amino acids such as polylysine and polyarginine, positively charged dendrimers and fractured dendrimers, cationic p-cyclodextrin containing polymers (CD-polymers), DEAE-dextran and the like. In certain embodiments, a reagent for the introduction of a nucleic acid into cells can comprise one or more lipids which can be cationic lipids and/or neutral lipids. Preferred lipids include, but are not limited to, N-[1-(2,3-Dioleyloxy)propyl]-N,N,N-trimethylammonium (DOTMA), dioleoyl phosphatidyl ethanolamine (DOPE), 1,2-Bis(oleoyloxy)-3-(trimethylammonio)propane (DOTAP), 1,2-Dioleoyl-3-myristoyl-sn-glycerol (DOTB), 1,2-dioleoyl-3-succinyl-sn-glycerol choline ester (DOSC), cholesterol (4′-trimethylammonio) butanoate (ChOTB), cetyltrimethylammonium bromide (CTAB), 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dimyristyloxypropyl-3-dimethyl-hydroxyethyl ammonium bromide (DMRIE), spermine conjugated to one or more lipids (for example, 5-carboxyspermylglycine dioctadecylamide), lipopolylysine (polylysine conjugated to DOPE), TRIS (Tris(hydroxymethyl)aminomethane, tromethamine) conjugated fatty acids (TFAs), and/or peptides such as trilysyl-alanyl-TRIS mono-, di-, and tri-palmitate, 3 beta [N-(N′, N′-dimethylaminoethane)-carbamoyl] cholesterol (DC-Chol), N-(alpha-trimethylammonioacetyl)-didodecyl-D-glutamate chloride (TMAG), dimethyl dioctadecylammonium bromide (DDAB), 2,3-dioleyloxy-N-[2(sperminecarboxamido)ethyl]-N,Ndimethyl-1-propanaminium trifluoroacetate (DOSPA), and combinations thereof. Other types of cationic lipids that have been used to introduce nucleic acids into cells include highly packed polycationic ammonium, sulfonium and phosphonium lipids such as those described in U.S. Pat. Nos. 5,674,908 and 5,834,439, and PCT Publication No. WO 00/27795. One transfection reagent for delivery of macromolecules is LIPOFECTAMINE 2000™ (see, PCT Publication No. WO 00/27795).

### Cell Expansion

In certain embodiments, prior to the introduction of one or more polynucleotides into the mammalian cells to generate AAV producer cells, the mammalian cells are expanded by culturing the mammalian cells in a suitable culture medium until a target cell density is reached. Cell density can be measured by any method known to those of skill in the art, for example, using a hemacytometer, an automated cell counting device, and flow-cytometry based methods. A suitable target cell density appropriate for introducing one or more polynucleotides to generate AAV producer cells includes, without limitation, a density of about 1E5 cells/mL to about 3E7 cells/mL of culture media, e.g., about 1E5 cells/mL, about 1.5E5 cells/mL, about 2E5 cells/mL, about 2.5E5 cells/mL, about 3E5 cells/mL, about 3.5E5 cells/mL, about 4E5 cells/mL, about 4.5E5 cells/mL, about 5E5 cells/mL, about 5.5E5 cells/mL, about 6E5 cells/mL, about 6.5E5 cells/mL, about 7E5 cells/mL, about 7.5E5 cells/mL, about 8E5 cells/mL, about 8.5E5 cells/mL, about 9E5 cells/mL, about 9.5E5 cells/mL, about 1E6 cells/mL, about 1.5E6 cells/mL, about 2E6 cells/mL, about 2.5E6 cells/mL, about 3E6 cells/mL, about 3.5E6 cells/mL, about 4E6 cells/mL, about 4.5E6 cells/mL, about 5E6 cells/mL, about 5.5E6 cells/mL, about 6E6 cells/mL, about 6.5E6 cells/mL, about 7E6 cells/mL, about 7.5E6 cells/mL, about 8E6 cells/mL, about 8.5E6 cells/mL, about 9E6 cells/mL, about 9.5E6 cells/mL, about 1E7 cells/mL, about 1.1E7 cells/mL, about 1.2E7 cells/mL, about 1.3E7 cells/mL, about 1.4E7 cells/mL, about 1.5E7 cells/mL, about 1.6E7 cells/mL, about 1.7E7 cells/mL, about 1.8E7 cells/mL, about 1.9E7 cells/mL, about 2E7 cells/mL, about 2.1E7 cells/mL, about 2.2E7 cells/mL, about 2.3E7 cells/mL, about 2.4E7 cells/mL, about 2.5E7 cells/mL, about 2.6E7 cells/mL, about 2.7E7 cells/mL, about 2.8E7 cells/mL, about 2.9E7 cells/mL, or about 3E7 cells/mL of culture media. In certain embodiments, the cell density appropriate for introducing one or more polynucleotides to generate AAV producer cells is about 1.8E6 cells/mL to about 2.4E6 cells/mL.

The mammalian cells may be grown to a target cell density in an appropriate expansion medium suitable for expanding the mammalian cells to the target cell density. The expansion medium may comprise any essential and non-essential amino acids, vitamins, lipids, and other components required for minimal growth and/or survival of a mammalian cell. In certain embodiments, the expansion medium comprises components that enhances the growth and/or survival of a mammalian cell above the minimal rate. Such components may include, without limitation, hormones and growth factors. In certain embodiments, the expansion medium comprises, without limitation, one or more of a salt, a carbohydrate (such as a sugar, e.g., glucose, galactose, maltose, and fructose), an amino acid, a vitamin (such as B group vitamins (e.g., B12), vitamin A, vitamin E, riboflavin, thiamine, and biotin), fatty acids and lipids (such as cholesterol and steroids), proteins and peptides (such as albumin, transferrin, fibronectin, and lutein), and trace elements (such as zinc, copper, selenium, and tricarboxylic acid intermediates). In certain embodiments, the expansion media includes serum (such as fetal bovine serum, newborn calf serum, horse serum, or human serum), hydrolysates (hydrolyzed proteins derived from plant or animal sources), and combinations thereof. The expansion media can comprise any component known to those of skill in the art that ensures at least a minimal growth and/or survival of a mammalian cell. In certain embodiments, the expansion medium is a defined medium or chemically defined medium. In certain embodiments, the expansion medium is a defined medium or chemically defined medium that is serum-free (i.e., contains no serum proteins, hydrolysates, or serum fractions).

Suitable expansion media include, without limitation, commercially available media such as CDM4HEK293 (Cytiva), DMEM/F-12 (ThermoFisher), CD OPTICHO™ (ThermoFisher), CD EFFICIENTFEED™ (ThermoFisher), EXPI293™ EXPRESSION MEDIUM (ThermoFisher), CELL BOOST® (Cytiva), BALANCD® CHO Feed (Irvine Scientific), BD RECHARGE® (Becton Dickinson), CELLVENTO@ Feed (EMD Millipore), EX-CELL CHOZN FEED® (Sigma-Aldrich), CHO Feed Bioreactor Supplement (Sigma-Aldrich), Sheff-CHO (Kerry), Zap-CHO (InVitria), ACTICHO™ (Cytiva), Ham's F10 (Sigma), Minimal Essential Medium ([MEM], Sigma), Roswell Park Memorial institute (RPMI)-1640 (Sigma), Dulbecco's Modified Eagle's Medium (DMEM, Sigma), and GIBCO™ Viral Production Medium (ThermoFisher), with or without supplementation with any of the aforementioned components. Suitable expansion media also includes, those described in Ham and Wallace, (1979) Meth. Enz., 58:44; Barnes and Sato, (1980) Anal. Biochem., 102:255; U.S. Pat. Nos. 4,767,704; 4,657,866; 4,927,762; 5,122,469 or 4,560,655; PCT Publication No. WO 90/03430; and WO 87/00195, see also, U.S. Published Application No. 20160289633.

### III. Recombinant Adeno-Associated Virus

The methods provided by the present disclosure are for the production of rAAV particles.

rAAV Genome

The rAAV particle comprises an rAAV genome. In certain embodiments, the rAAV genome comprises a transgene.

In certain embodiments, the transgene comprises one or more sequences encoding an RNA molecule. Suitable RNA molecules include, without limitation, miRNA, shRNA, siRNA, antisense RNA, gRNA, antagomirs, miRNA sponges, RNA aptazymes, RNA aptamers, mRNA, lncRNAs, ribozymes, and synthetic RNAs known in the art.

In certain embodiments, the transgene encodes one or more polypeptides, or a fragment thereof. Such transgenes can comprise the complete coding sequence of a polypeptide, or only a fragment of a coding sequence of a polypeptide. In certain embodiments, the transgene encodes a polypeptide that is useful to treat a disease or disorder in a subject. Suitable polypeptides include, without limitation, β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β), and the like; soluble receptors, such as soluble TNF-a receptors, soluble interleukin receptors (e.g., soluble IL-1 receptors and soluble type II IL-1 receptors), soluble γ/Δ T cell receptors, ligand-binding fragments of a soluble receptor, and the like; enzymes, such as a-glucosidase, imiglucerase, β-glucocerebrosidase, and alglucerase; enzyme activators, such as tissue plasminogen activator; chemokines, such as IP-10, monokine induced by interferon-gamma (Mig), Groα/IL-8, RANTES, MIP-la, MIP-10, MCP-1, PF-4, and the like; angiogenic agents, such as vascular endothelial growth factors (VEGFs, e.g., VEGF121, VEGF165, VEGF-C, VEGF-2), glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastrin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); tissue factors; macrophage activating factors; tumor necrosis factor (TNF); neutrophil chemotactic factor (NCF); tissue inhibitors of metalloproteinases; vasoactive intestinal peptide; angiogenin; angiotrophin; fibrin; hirudin; IL-1 receptor antagonists; ciliary neurotrophic factor (CNTF); brain-derived neurotrophic factor (BDNF); neurotrophins 3 and 4/5 (NT-3 and -4/5); glial cell derived neurotrophic factor (GDNF); aromatic amino acid decarboxylase (AADC); Factor VIII, Factor IX, Factor X; dystrophin or mini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase, glucose transporter, aldolase A, β-enolase, glycogen synthase; lysosomal enzymes, such as iduronate-2-sulfatase (12S), and arylsulfatase A; and mitochondrial proteins, such as frataxin. In certain embodiments, the transgene encodes a polypeptide which is not selected from the group consisting of phenylalanine hydroxylase (PAH), arylsulfatase A (ARSA), iduronate 2-sulfatase (I2S), and an anti-complement component 5 (C5) antibody.

In certain embodiments, the transgene encodes a protein that may be defective in one or more lysosomal storage diseases. Suitable proteins include, without limitation, α-sialidase, cathepsin A, α-mannosidase, β-mannosidase, glycosylasparaginase, α-fucosidase, α-N-acetylglucosaminidase, β-galactosidase, β-hexosaminidase α-subunit, β-hexosaminidase β-subunit, GM2 activator protein, glucocerebrosidase, Saposin C, Arylsulfatase A, Saposin B, formyl-glycine generating enzyme, β-galactosylceramidase, α-galactosidase A, iduronate sulfatase, α-iduronidase, heparan N-sulfatase, acetyl-CoA transferase, N-acetyl glucosaminidase, β-glucuronidase, N-acetyl glucosamine 6-sulfatase, N-acetylgalactosamine 4-sulfatase, galactose 6-sulfatase, hyaluronidase, α-glucosidase, acid sphingomyelinase, acid ceramidase, acid lipase, cathepsin K, tripeptidyl peptidase, palmitoyl-protein thioesterase, cystinosin, sialin, UDP-N-acetylglucosamine, phosphotransferase γ-subunit, mucolipin-1, LAMP-2, NPC1, CLN3, CLN 6, CLN 8, LYST, MYOV, RAB27A, melanophilin, and AP3 β-subunit. In certain embodiments, the transgene does not encode arylsulfatase A (ARSA).

In certain embodiments, the transgene encodes a protein selected from the group consisting of iduronate-2-sulfatase (I2S), frataxin (FXN), glucose-6-phosphatase (G6Pase), phosphoenolpyruvate carboxykinase (PEPCK), cyclin-dependent kinase-like 5 (CDKL5/STK9), galactose-1 phosphate uridyltransferase, phenylalanine hydroxylase (PAH), branched-chain alpha-keto acid dehydrogenase, fumarylacetoacetate hydrolase, methylmalonyl-CoA mutase, medium-chain acyl-CoA dehydrogenase, ornithine transcarbamylase (OTC), argininosuccinic acid synthetase (ASS1), low density lipoprotein receptor (LDLR) protein, UDP-glucuronosyltransferase, adenosine deaminase, hypoxanthine guanine phosphoribosyltransferase, biotinidase, alpha-galactosidase A, copper-transporting ATPase 2 (ATP7B), beta-glucocerebrosidase, 70 kDa peroxisomal membrane protein (PMP70), and arylsulfatase A (ARSA). In certain embodiments, the transgene encodes a protein which is not selected from the group consisting of phenylalanine hydroxylase (PAH), arylsulfatase A (ARSA), iduronate 2-sulfatase (12S), and an anti-complement component 5 (C5) antibody.

In certain embodiments, the transgene encodes an antibody or a fragment thereof (e.g., a Fab, scFv, or full-length antibody). Suitable antibodies include, without limitation, muromonab-cd3, efalizumab, tositumomab, daclizumab, nebacumab, catumaxomab, edrecolomab, abciximab, rituximab, basiliximab, palivizumab, infliximab, trastuzumab, adalimumab, ibritumomab tiuxetan, omalizumab, cetuximab, bevacizumab, natalizumab, panitumumab, ranibizumab, eculizumab, certolizumab, ustekinumab, canakinumab, golimumab, ofatumumab, tocilizumab, denosumab, belimumab, ipilimumab, brentuximab vedotin, pertuzumab, raxibacumab, obinutuzumab, alemtuzumab, siltuximab, ramucirumab, vedolizumab, blinatumomab, nivolumab, pembrolizumab, idarucizumab, necitumumab, dinutuximab, secukinumab, mepolizumab, alirocumab, evolocumab, daratumumab, elotuzumab, ixekizumab, reslizumab, olaratumab, bezlotoxumab, atezolizumab, obiltoxaximab, inotuzumab ozogamicin, brodalumab, guselkumab, dupilumab, sarilumab, avelumab, ocrelizumab, emicizumab, benralizumab, gemtuzumab ozogamicin, durvalumab, burosumab, erenumab, galcanezumab, lanadelumab, mogamulizumab, tildrakizumab, cemiplimab, fremanezumab, ravulizumab, emapalumab, ibalizumab, moxetumomab, caplacizumab, romosozumab, risankizumab, polatuzumab, eptinezumab, leronlimab, sacituzumab, brolucizumab, isatuximab, and teprotumumab.

In certain embodiments, the transgene encodes a nuclease. Suitable nucleases include, without limitation, zinc fingers nucleases (ZFN) (see, e.g., Porteus, and Baltimore (2003) Science 300: 763; Miller et al. (2007) Nat. Biotechnol. 25:778-785; Sander et al. (2011) Nature Methods 8:67-69; and Wood et al. (2011) Science 333:307, each of which is hereby incorporated by reference in its entirety), transcription activator-like effectors nucleases (TALEN) (see, e.g., Wood et al. (2011) Science 333:307; Boch et al. (2009) Science 326:1509-1512; Moscou and Bogdanove (2009) Science 326:1501; Christian et al. (2010) Genetics 186:757-761; Miller et al. (2011) Nat. Biotechnol. 29:143-148; Zhang et al. (2011) Nat. Biotechnol. 29:149-153; and Reyon et al. (2012) Nat. Biotechnol. 30(5): 460-465, each of which is hereby incorporated by reference in its entirety), homing endonucleases, meganucleases (see, e.g., U.S. Patent Publication No. US 2014/0121115, which is hereby incorporated by reference in its entirety), and RNA-guided nucleases (see, e.g., Makarova et al. (2018) The CRISPR Journal 1(5): 325-336; and Adli (2018) Nat. Communications 9:1911, each of which is hereby incorporated by reference in its entirety).

In certain embodiments, the transgene encodes an RNA-guided nuclease. Suitable RNA-guided nucleases include, without limitation, Class I and Class II clustered regularly interspaced short palindromic repeats (CRISPR)-associated nucleases. Class I is divided into types I, III, and IV, and includes, without limitation, type I (Cas3), type I-A (Cas8a, Cas5), type I-B (Cas8b), type I-C (Cas8c), type 1-D (Cas10d), type I-E (Cse1, Cse2), type I-F (Csy1, Csy2, Csy3), type I-U (GSU0054), type III (Cas10), type III-A (Csm2), type III-B (Cmr5), type III-C (Csx10 or Csx11), type III-D (Csx10), and type IV (Csf1). Class II is divided into types II, V, and VI, and includes, without limitation, type II (Cas9), type II-A (Csn2), type II-B (Cas4), type V (Cpf1, C2c1, C2c3), and type VI (Cas13a, Cas13b, Cas13c). RNA-guided nucleases also include naturally-occurring Class II CRISPR nucleases such as Cas9 (Type II) or Cas12a/Cpf1 (Type V), as well as other nucleases derived or obtained therefrom. Exemplary Cas9 nucleases that may be used in the present invention include, but are not limited to, S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9).

In certain embodiments, the transgene encodes one or more reporter sequences, which upon expression produce a detectable signal. Such reporter sequences include, without limitation, DNA sequences encoding β-lactamase, β-galactosidase (LacZ), alkaline phosphatase, thymidine kinase, green fluorescent protein (GFP), red fluorescent protein (RFP), chloramphenicol acetyltransferase (CAT), luciferase, membrane bound proteins, including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

In certain embodiments, the rAAV genome comprises a transcriptional regulatory element (TRE) operably linked to the transgene, to control expression of an RNA or polypeptide encoded by the transgene. In certain embodiments, the TRE comprises a constitutive promoter. In certain embodiments, the TRE can be active in any mammalian cell (e.g., any human cell). In certain embodiments, the TRE is active in a broad range of human cells. Such TREs may comprise constitutive promoter and/or enhancer elements, including any of those described herein, and any of those known to one of skill in the art. In certain embodiments, the TRE comprises an inducible promoter. In certain embodiments, the TRE may be a tissue-specific TRE, i.e., it is active in specific tissue(s) and/or organ(s). A tissue-specific TRE comprises one or more tissue-specific promoter and/or enhancer elements, and optionally one or more constitutive promoter and/or enhancer elements. A skilled artisan would appreciate that tissue-specific promoter and/or enhancer elements can be isolated from genes specifically expressed in the tissue by methods well known in the art.

Suitable promoters include, e.g., cytomegalovirus promoter (CMV) (Stinski et al. (1985) Journal of Virology 55(2): 431-441), CMV early enhancer/chicken β-actin (CBA) promoter/rabbit β-globin intron (CAG) (Miyazaki et al. (1989) Gene 79(2): 269-277), CB^{SB} (Jacobson et al. (2006) Molecular Therapy 13(6): 1074-1084), human elongation factor 1α promoter (EF1α) (Kim et al. (1990) Gene 91 (2): 217-223), human phosphoglycerate kinase promoter (PGK) (Singer-Sam et al. (1984) Gene 32(3): 409-417), mitochondrial heavy-strand promoter (Lodeiro et al. (2012) PNAS 109(17): 6513-6518), ubiquitin promoter (Wulff et al. (1990) FEBS Letters 261: 101-105). In certain embodiments, the TRE comprises a cytomegalovirus (CMV) promoter/enhancer (e.g., comprising a nucleotide sequence at least 90%, 91%, 92%, 93%, 94%, 95%, 96%, 97%, 98%, 99%, or 100% identical to SEQ ID NO: 18 or 19), an SV40 promoter, a chicken beta actin (CBA) promoter (e.g., comprising a nucleotide sequence at least 90%, 91%, 92%, 93%, 94%, 95%, 96%, 97%, 98%, 99%, or 100% identical to SEQ ID NO: 20 or 21), an smCBA promoter (e.g., comprising a nucleotide sequence at least 90%, 91%, 92%, 93%, 94%, 95%, 96%, 97%, 98%, 99%, or 100% identical to SEQ ID NO: 22), a human elongation factor 1 alpha (EF1α) promoter (e.g., comprising a nucleotide sequence at least 90%, 91%, 92%, 93%, 94%, 95%, 96%, 97%, 98%, 99%, or 100% identical to SEQ ID NO: 23), a minute virus of mouse (MVM) intron which comprises transcription factor binding sites (e.g., comprising a nucleotide sequence at least 90%, 91%, 92%, 93%, 94%, 95%, 96%, 97%, 98%, 99%, or 100% identical to SEQ ID NO: 24 or 25), a human phosphoglycerate kinase (PGK1) promoter, a human ubiquitin C (Ubc) promoter, a human beta actin promoter, a human neuron-specific enolase (ENO2) promoter, a human beta-glucuronidase (GUSB) promoter, a rabbit beta-globin element (e.g., comprising a nucleotide sequence at least 90%, 91%, 92%, 93%, 94%, 95%, 96%, 97%, 98%, 99%, or 100% identical to SEQ ID NO: 26 or 27), a human calmodulin 1 (CALM1) promoter (e.g., comprising a nucleotide sequence at least 90%, 91%, 92%, 93%, 94%, 95%, 96%, 97%, 98%, 99%, or 100% identical to SEQ ID NO: 28), a human ApoE/C-I hepatic control region (HCR1) (e.g., comprising a nucleotide sequence at least 90%, 91%, 92%, 93%, 94%, 95%, 96%, 97%, 98%, 99%, or 100% identical to SEQ ID NO: 29), a human al-antitrypsin (hAAT) promoter (e.g., comprising a nucleotide sequence at least 90%, 91%, 92%, 93%, 94%, 95%, 96%, 97%, 98%, 99%, or 100% identical to SEQ ID NO: 30, 31, or 32), an extended HCR1 (e.g., comprising a nucleotide sequence at least 90%, 91%, 92%, 93%, 94%, 95%, 96%, 97%, 98%, 99%, or 100% identical to SEQ ID NO: 33), an HS-CRM8 element of an hAAT promoter (e.g., comprising a nucleotide sequence at least 90%, 91%, 92%, 93%, 94%, 95%, 96%, 97%, 98%, 99%, or 100% identical to SEQ ID NO: 34), a human transthyretin (TTR) promoter (e.g., comprising a nucleotide sequence at least 90%, 91%, 92%, 93%, 94%, 95%, 96%, 97%, 98%, 99%, or 100% identical to SEQ ID NO: 35), and/or a human Methyl-CpG Binding Protein 2 (MeCP2) promoter. Any of the TREs described herein can be combined in any order to drive efficient transcription. For example, an rAAV genome may comprise a TRE comprising a CMV enhancer, a CBA promoter, and the splice acceptor from exon 3 of the rabbit beta-globin gene, collectively called a CAG promoter (e.g., comprising a nucleotide sequence at least 90%, 91%, 92%, 93%, 94%, 95%, 96%, 97%, 98%, 99%, or 100% identical to SEQ ID NO: 36). For example, an rAAV genome may comprise a TRE comprising a hybrid of CMV enhancer and CBA promoter followed by a splice donor and splice acceptor, collectively called a CASI promoter region (e.g., comprising a nucleotide sequence at least 90%, 91%, 92%, 93%, 94%, 95%, 96%, 97%, 98%, 99%, or 100% identical to SEQ ID NO: 37). For example, an rAAV genome may comprise a TRE comprising an HCR1 and hAAT promoter (also referred to as an LP1 promoter, e.g., comprising a nucleotide sequence at least 90%, 91%, 92%, 93%, 94%, 95%, 96%, 97%, 98%, 99%, or 100% identical to SEQ ID NO: 38).

In certain embodiments, the TRE is brain-specific (e.g., neuron-specific, glial cell-specific, astrocyte-specific, oligodendrocyte-specific, microglia-specific and/or central nervous system-specific). Exemplary brain-specific TREs may comprise one or more elements from, without limitation, human glial fibrillary acidic protein (GFAP) promoter, human synapsin 1 (SYN1) promoter, human synapsin 2 (SYN2) promoter, human metallothionein 3 (MT3) promoter, and/or human proteolipid protein 1 (PLP1) promoter. More brain-specific promoter elements are disclosed in WO 2016/100575A1, which is incorporated by reference herein in its entirety.

In certain embodiments, the native promoter for the transgene may be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In certain embodiments, the rAAV genome comprises an editing genome. Editing genomes can be used to edit the genome of a cell by homologous recombination of the editing genome with a genomic region surrounding a target locus in the cell. In certain embodiments, the editing genome is designed to correct a genetic defect in a gene by homologous recombination. Editing genomes generally comprise: (i) an editing element for editing a target locus in a target gene, (ii) a 5′ homology arm nucleotide sequence 5′ of the editing element having homology to a first genomic region 5′ to the target locus, and (iii) a 3′ homology arm nucleotide sequence 3′ of the editing element having homology to a second genomic region 3′ to the target locus, wherein the portion of the editing genome comprising the 5′ homology arm, editing element, and 3′ homology arm can be in the sense or antisense orientation relative to the target locus. Suitable target genes for editing using an editing genome include, without limitation, phenylalanine hydroxylase (PAH), cystic fibrosis conductance transmembrane regulator (CFTR), beta hemoglobin (HBB), oculocutaneous albinism II (OCA2), Huntingtin (HTT), dystrophia myotonica-protein kinase (DMPK), low-density lipoprotein receptor (LDLR), apolipoprotein B (APOB), neurofibromin 1 (NF1), polycystic kidney disease 1 (PKD1), polycystic kidney disease 2 (PKD2), coagulation factor VIII (F8), dystrophin (DMD), phosphate-regulating endopeptidase homologue, X-linked (PHEX), methyl-CpG-binding protein 2 (MECP2), and ubiquitin-specific peptidase 9Y, Y-linked (USP9Y).

In certain embodiments, the rAAV genomes disclosed herein further comprise a transcription terminator (e.g., a polyadenylation sequence). In certain embodiments, the transcription terminator is 3′ to the transgene. The transcription terminator may be any sequence that effectively terminates transcription, and a skilled artisan would appreciate that such sequences can be isolated from any genes that are expressed in the cell in which transcription of the at least a portion of an antibody coding sequence is desired. In certain embodiments, the transcription terminator comprises a polyadenylation sequence. In certain embodiments, the polyadenylation sequence is identical or substantially identical to the endogenous polyadenylation sequence of an immunoglobulin gene. In certain embodiments, the polyadenylation sequence is an exogenous polyadenylation sequence. In certain embodiments, the polyadenylation sequence is an SV40 polyadenylation sequence (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 14, 47, or 48, or a nucleotide sequence complementary thereto). In certain embodiments, the polyadenylation sequence comprises the nucleotide sequence set forth in SEQ ID NO: 14. In certain embodiments, the polyadenylation sequence consists of the nucleotide sequence set forth in SEQ ID NO: 14. In certain embodiments, the polyadenylation sequence is a bovine growth hormone (BGH) polyadenylation sequence (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 49, or a nucleotide sequence complementary thereto). In certain embodiments, the polyadenylation sequence comprises the nucleotide sequence set forth in SEQ ID NO: 49. In certain embodiments, the polyadenylation sequence consists of the nucleotide sequence set forth in SEQ ID NO: 49.

In certain embodiments, the rAAV genome comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, or 54. In certain embodiments, the editing element comprises the nucleotide sequence set forth in SEQ ID NO: 50, 51, 52, 53, or 54. In certain embodiments, the editing element consists of the nucleotide sequence set forth in SEQ ID NO: 50, 51, 52, 53, or 54.

In certain embodiments, the rAAV genomes disclosed herein further comprise a 5′ inverted terminal repeat (5′ ITR) nucleotide sequence 5′ of the TRE, and a 3′ inverted terminal repeat (3′ ITR) nucleotide sequence 3′ of the polyadenylation sequence associated with an antibody light chain coding sequence. ITR sequences from any AAV serotype or variant thereof can be used in the rAAV genomes disclosed herein. The 5′ and 3′ ITR can be from an AAV of the same serotype or from AAVs of different serotypes. Exemplary ITRs for use in the rAAV genomes disclosed herein are set forth in SEQ ID NOs: 39, 40, 41, 42, 43, and 44, herein.

In certain embodiments, the 5′ ITR or 3′ ITR is from AAV2. In certain embodiments, both the 5′ ITR and the 3′ ITR are from AAV2. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 39, or the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 40. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 39, and the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 40. In certain embodiments, the rAAV genome comprises a 5′ ITR nucleotide sequence having the sequence of SEQ ID NO: 39, and a 3′ ITR nucleotide sequence having the sequence of SEQ ID NO: 40.

In certain embodiments, the 5′ ITR or 3′ ITR are from AAV5. In certain embodiments, both the 5′ ITR and 3′ ITR are from AAV5. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 42, or the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 43. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 42, and the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 43. In certain embodiments, the rAAV genome comprises a 5′ ITR nucleotide sequence having the sequence of SEQ ID NO: 42, and a 3′ ITR nucleotide sequence having the sequence of SEQ ID NO: 43.

In certain embodiments, the 5′ ITR nucleotide sequence and the 3′ ITR nucleotide sequence are substantially complementary to each other (e.g., are complementary to each other except for mismatch at 1, 2, 3, 4, or 5 nucleotide positions in the 5′ or 3′ ITR).

In certain embodiments, the 5′ ITR or the 3′ ITR is modified to reduce or abolish resolution by Rep protein (“non-resolvable ITR”). In certain embodiments, the non-resolvable ITR comprises an insertion, deletion, or substitution in the nucleotide sequence of the terminal resolution site. Such modification allows formation of a self-complementary, double-stranded DNA genome of the AAV after the rAAV genome is replicated in an infected cell. Exemplary non-resolvable ITR sequences are known in the art (see, e.g., those provided in U.S. Pat. Nos. 7,790,154 and 9,783,824, which are incorporated by reference herein in their entirety). In certain embodiments, the 5′ ITR comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 41. In certain embodiments, the 5′ ITR consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 41. In certain embodiments, the 5′ ITR consists of the nucleotide sequence set forth in SEQ ID NO: 41. In certain embodiments, the 3′ ITR comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 44. In certain embodiments, the 5′ ITR consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 44. In certain embodiments, the 3′ ITR consists of the nucleotide sequence set forth in SEQ ID NO: 44. In certain embodiments, the 5′ ITR consists of the nucleotide sequence set forth in SEQ ID NO: 41, and the 3′ ITR consists of the nucleotide sequence set forth in SEQ ID NO: 44. In certain embodiments, the 5′ ITR consists of the nucleotide sequence set forth in SEQ ID NO: 41, and the 3′ ITR consists of the nucleotide sequence set forth in SEQ ID NO: 44.

In certain embodiments, the 5′ ITR is flanked by an additional nucleotide sequence derived from a wild-type AAV2 genomic sequence. In certain embodiments, the 5′ ITR is flanked by an additional 46 bp sequence derived from a wild-type AAV2 sequence that is adjacent to a wild-type AAV2 ITR in an AAV2 genome. In certain embodiments, the additional 46 bp sequence is 3′ to the 5′ ITR in the rAAV genome. In certain embodiments, the 46 bp sequence consists of the nucleotide sequence set forth in SEQ ID NO: 45.

In certain embodiments, the 3′ ITR is flanked by an additional nucleotide sequence derived from a wild-type AAV2 genomic sequence. In certain embodiments, the 3′ ITR is flanked by an additional 37 bp sequence derived from a wild-type AAV2 sequence that is adjacent to a wild-type AAV2 ITR in an AAV2 genome. See, e.g., Savy et al. Human Gene Therapy Methods (2017) 28(5): 277-289 (which is hereby incorporated by reference herein in its entirety). In certain embodiments, the additional 37 bp sequence is 5′ to the 3′ ITR in the rAAV genome. In certain embodiments, the 37 bp sequence consists of the nucleotide sequence set forth in SEQ ID NO: 46.

In certain embodiments, the rAAV genome comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 55, 56, 57, 58, or 59. In certain embodiments, the editing element comprises the nucleotide sequence set forth in SEQ ID NO: 55, 56, 57, 58, or 59. In certain embodiments, the editing element consists of the nucleotide sequence set forth in SEQ ID NO: 55, 56, 57, 58, or 59.

### AAV Capsid Protein

In certain embodiments, the rAAV comprises an AAV capsid. In certain embodiments, the AAV capsid comprises an AAV capsid protein. The rAAV can comprise an AAV capsid comprising an AAV capsid protein from any AAV capsid known in the art, including natural AAV isolates and variants thereof.

AAV capsid proteins include VP1, VP2, and VP3 capsid proteins. VP1, VP2, and/or VP3 capsid proteins assemble into a capsid that surrounds the rAAV genome. In certain embodiments, assembly of the capsid proteins is facilitated by the assembly-activating protein (AAP). Capsids of certain AAV serotypes require the role of AAP in transporting the capsid proteins to the nucleolus for assembly. For example, AAV1, AAV2, AAV3, AAV6, AAV7, AAV8, AAV9, AAV10, and AAV12 require AAP to form capsids, while capsids of AAV4, AAV5, and AAV11 can assemble without AAP. See, e.g., Earley et al. (2017) J. Virol. 91(3): e01980-16.

Different AAV serotypes or variants thereof comprise AAV capsid proteins having different amino acid sequences. Suitable AAV capsid proteins include, without limitation, a capsid protein from AAV1, AAV2, AAV3, AAV4, AAV5, AAV6, AAV7, AAV8, AAV9, AAV10, AAV11, AAV12, AAV13, AAV-DJ, AAV-LK03, NP59, VOY101, VOY201, VOY701, VOY801, VOY1101, AAVPHP.N, AAVPHP.A, AAVPHP.B, PHP.B2, PHP.B3, G2A3, G2B4, G2B5, PHP.S, AAVrh74, AAVrh10, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSCl2, AAVHSC13, AAVHSCl4, AAVHSC15, AAVHSC16, AAVHSC17, and any variants thereof. The sequences of the various AAV capsid proteins are disclosed in, e.g., U.S. Patent Publication Nos.: US20140359799, US20150376607, US20150159173, US20170081680, and US20170360962A1, and PCT Publication No. WO 2020227515, the disclosures of which are incorporated by reference herein in their entireties.

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 203-736 of SEQ ID NO: 1, 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 203-736 of SEQ ID NO: 1, 2, 3, 4, 5, 6, 7, 8, 9, 10, 11, 12, 13, 15, 16, or 17, wherein: the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 203-736 of SEQ ID NO: 1, 2, 3, 4, 5, 6, 7, 8, 9, 10, 11, 12, 13, 15, 16, or 17.

In certain embodiments, the capsid protein does not comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 203-736 of SEQ ID NO: 1, 2, 3, 4, 5, 6, 7, 8, 9, 10, 11, 12, 13, 15, 16, or 17. In certain embodiments, the capsid protein does not comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 203-736 of SEQ ID NO: 1, 2, 3, 4, 5, 6, 7, 8, 9, 10, 11, 12, 13, 15, 16, or 17, wherein: the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the capsid protein does not comprise the amino acid sequence of amino acids 203-736 of SEQ ID NO: 1, 2, 3, 4, 5, 6, 7, 8, 9, 10, 11, 12, 13, 15, 16, or 17.

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 138-736 of SEQ ID NO: 1, 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 138-736 of SEQ ID NO: 1, 2, 3, 4, 5, 6, 7, 8, 9, 10, 11, 12, 13, 15, 16, or 17, wherein: the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 138-736 of SEQ ID NO: 1, 2, 3, 4, 5, 6, 7, 8, 9, 10, 11, 12, 13, 15, 16, or 17.

In certain embodiments, the capsid protein does not comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 138-736 of SEQ ID NO: 1, 2, 3, 4, 5, 6, 7, 8, 9, 10, 11, 12, 13, 15, 16, or 17. In certain embodiments, the capsid protein does not comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 138-736 of SEQ ID NO: 1, 2, 3, 4, 5, 6, 7, 8, 9, 10, 11, 12, 13, 15, 16, or 17, wherein: the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the capsid protein does not comprise the amino acid sequence of amino acids 138-736 of SEQ ID NO: 1, 2, 3, 4, 5, 6, 7, 8, 9, 10, 11, 12, 13, 15, 16, or 17.

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 1-736 of SEQ ID NO: 1, 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 1-736 of SEQ ID NO: 1, 2, 3, 4, 5, 6, 7, 8, 9, 10, 11, 12, 13, 15, 16, or 17, wherein: the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 2 of SEQ ID NO: 16 is T, and the amino acid in the capsid protein corresponding to amino acid 312 of SEQ ID NO: 16 is Q. In certain embodiments, the amino acid in the capsid protein corresponding to amino acid 65 of SEQ ID NO: 16 is I, and the amino acid in the capsid protein corresponding to amino acid 626 of SEQ ID NO: 16 is Y. In certain embodiments, the amino acid in the capsid protein corresponding to amino acid 77 of SEQ ID NO: 16 is R, and the amino acid in the capsid protein corresponding to amino acid 690 of SEQ ID NO: 16 is K. In certain embodiments, the amino acid in the capsid protein corresponding to amino acid 119 of SEQ ID NO: 16 is L, and the amino acid in the capsid protein corresponding to amino acid 468 of SEQ ID NO: 16 is S. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 1-736 of SEQ ID NO: 1, 2, 3, 4, 5, 6, 7, 8, 9, 10, 11, 12, 13, 15, 16, or 17.

In certain embodiments, the capsid protein does not comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 1-736 of SEQ ID NO: 1, 2, 3, 4, 5, 6, 7, 8, 9, 10, 11, 12, 13, 15, 16, or 17. In certain embodiments, the capsid protein does not comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 1-736 of SEQ ID NO: 1, 2, 3, 4, 5, 6, 7, 8, 9, 10, 11, 12, 13, 15, 16, or 17, wherein: the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the capsid protein does not comprise the amino acid sequence of amino acids 1-736 of SEQ ID NO: 1, 2, 3, 4, 5, 6, 7, 8, 9, 10, 11, 12, 13, 15, 16, or 17.

In certain embodiments, the AAV capsid comprises two or more of: (a) a capsid protein comprising the amino acid sequence of amino acids 203-736 of SEQ ID NO: 1, 2, 3, 4, 6, 7, 10, 11, 12, 13, 15, 16, or 17; (b) a capsid protein comprising the amino acid sequence of amino acids 138-736 of SEQ ID NO: 1, 2, 3, 4, 5, 6, 7, 9, 10, 11, 12, 13, 15, 16, or 17; and (c) a capsid protein comprising the amino acid sequence of amino acids 1-736 of SEQ ID NO: 1, 2, 3, 4, 5, 6, 7, 8, 9, 10, 11, 12, 13, 15, 16, or 17. In certain embodiments, the AAV capsid comprises: (a) a capsid protein having an amino acid sequence consisting of amino acids 203-736 of SEQ ID NO: 1, 2, 3, 4, 6, 7, 10, 11, 12, 13, 15, 16, or 17; (b) a capsid protein having an amino acid sequence consisting of amino acids 138-736 of SEQ ID NO: 1, 2, 3, 4, 5, 6, 7, 9, 10, 11, 12, 13, 15, 16, or 17; and (c) a capsid protein having an amino acid sequence consisting of amino acids 1-736 of SEQ ID NO: 1, 2, 3, 4, 5, 6, 7, 8, 9, 10, 11, 12, 13, 15, 16, or 17.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 203-736 of SEQ ID NO: 8; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 138-736 of SEQ ID NO: 8; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 1-736 of SEQ ID NO: 8. In certain embodiments, the AAV capsid comprises one or more of: (a) a capsid protein comprising the amino acid sequence of amino acids 203-736 of SEQ ID NO: 8; (b) a capsid protein comprising the amino acid sequence of amino acids 138-736 of SEQ ID NO: 8; and (c) a capsid protein comprising the amino acid sequence of amino acids 1-736 of SEQ ID NO: 8. In certain embodiments, the AAV capsid comprises two or more of: (a) a capsid protein comprising the amino acid sequence of amino acids 203-736 of SEQ ID NO: 8; (b) a capsid protein comprising the amino acid sequence of amino acids 138-736 of SEQ ID NO: 8; and (c) a capsid protein comprising the amino acid sequence of amino acids 1-736 of SEQ ID NO: 8. In certain embodiments, the AAV capsid comprises: (a) a capsid protein having an amino acid sequence consisting of amino acids 203-736 of SEQ ID NO: 8; (b) a capsid protein having an amino acid sequence consisting of amino acids 138-736 of SEQ ID NO: 8; and (c) a capsid protein having an amino acid sequence consisting of amino acids 1-736 of SEQ ID NO: 8.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 203-736 of SEQ ID NO: 11; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 138-736 of SEQ ID NO: 11; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 1-736 of SEQ ID NO: 11. In certain embodiments, the AAV capsid comprises one or more of: (a) a capsid protein comprising the amino acid sequence of amino acids 203-736 of SEQ ID NO: 11; (b) a capsid protein comprising the amino acid sequence of amino acids 138-736 of SEQ ID NO: 11; and (c) a capsid protein comprising the amino acid sequence of amino acids 1-736 of SEQ ID NO: 11. In certain embodiments, the AAV capsid comprises two or more of: (a) a capsid protein comprising the amino acid sequence of amino acids 203-736 of SEQ ID NO: 11; (b) a capsid protein comprising the amino acid sequence of amino acids 138-736 of SEQ ID NO: 11; and (c) a capsid protein comprising the amino acid sequence of amino acids 1-736 of SEQ ID NO: 11. In certain embodiments, the AAV capsid comprises: (a) a capsid protein having an amino acid sequence consisting of amino acids 203-736 of SEQ ID NO: 11; (b) a capsid protein having an amino acid sequence consisting of amino acids 138-736 of SEQ ID NO: 11; and (c) a capsid protein having an amino acid sequence consisting of amino acids 1-736 of SEQ ID NO: 11.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 203-736 of SEQ ID NO: 13; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 138-736 of SEQ ID NO: 13; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 1-736 of SEQ ID NO: 13. In certain embodiments, the AAV capsid comprises one or more of: (a) a capsid protein comprising the amino acid sequence of amino acids 203-736 of SEQ ID NO: 13; (b) a capsid protein comprising the amino acid sequence of amino acids 138-736 of SEQ ID NO: 13; and (c) a capsid protein comprising the amino acid sequence of amino acids 1-736 of SEQ ID NO: 13. In certain embodiments, the AAV capsid comprises two or more of: (a) a capsid protein comprising the amino acid sequence of amino acids 203-736 of SEQ ID NO: 13; (b) a capsid protein comprising the amino acid sequence of amino acids 138-736 of SEQ ID NO: 13; and (c) a capsid protein comprising the amino acid sequence of amino acids 1-736 of SEQ ID NO: 13. In certain embodiments, the AAV capsid comprises: (a) a capsid protein having an amino acid sequence consisting of amino acids 203-736 of SEQ ID NO: 13; (b) a capsid protein having an amino acid sequence consisting of amino acids 138-736 of SEQ ID NO: 13; and (c) a capsid protein having an amino acid sequence consisting of amino acids 1-736 of SEQ ID NO: 13.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 203-736 of SEQ ID NO: 16; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 138-736 of SEQ ID NO: 16; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 1-736 of SEQ ID NO: 16. In certain embodiments, the AAV capsid comprises one or more of: (a) a capsid protein comprising the amino acid sequence of amino acids 203-736 of SEQ ID NO: 16; (b) a capsid protein comprising the amino acid sequence of amino acids 138-736 of SEQ ID NO: 16; and (c) a capsid protein comprising the amino acid sequence of amino acids 1-736 of SEQ ID NO: 16. In certain embodiments, the AAV capsid comprises two or more of: (a) a capsid protein comprising the amino acid sequence of amino acids 203-736 of SEQ ID NO: 16; (b) a capsid protein comprising the amino acid sequence of amino acids 138-736 of SEQ ID NO: 16; and (c) a capsid protein comprising the amino acid sequence of amino acids 1-736 of SEQ ID NO: 16. In certain embodiments, the AAV capsid comprises: (a) a capsid protein having an amino acid sequence consisting of amino acids 203-736 of SEQ ID NO: 16; (b) a capsid protein having an amino acid sequence consisting of amino acids 138-736 of SEQ ID NO: 16; and (c) a capsid protein having an amino acid sequence consisting of amino acids 1-736 of SEQ ID NO: 16.

### EXAMPLES

The following examples are offered by way of illustration, and not by way of limitation.

### Example 1: Optimization of Upstream Process: Post-Transfection Conditions

In order to investigate the impact of post-transfection parameters on the productivity of a representative AAV particle, various post-transfection bioreactor setpoint parameters were tested to maximize the resulting AAV vector genome and viral capsid yields.

For each parameter tested, HEK293 cells were expanded by shake flask culture with a target seed density of >0.25E6 viable cells/mL and a target final density of <6E6 viable cells/mL. Expanded cells were inoculated into an Applikon 3 L Glass Bioreactor containing the appropriate amount of cell culture medium prior to transfection at a 2 L working volume. The setpoints for the bioreactor growth and lysis phases are set forth in Table 1.

TABLE 1
Bioreactor growth and lysis setpoints

| Parameter | Full fill growth | Lysis |
|---|---|---|
| Temperature (° C.) | 37 | 37 |
| Agitation (RPM) | 263 | 285 |
| P/V (W/m³) | 23.8 | 26.3 |
| pH | 7.20 ± 0.10 | Off |
| Dissolved oxygen (DO; %) | 30 | Off |

Cell transfection was performed generally as described in Grieger et al. (2016) Molecular Therapy 24(2): 287-297. Briefly, transfection mixes were prepared by mixing calculated volumes of vector(s), transfection media, and polyethylenimine (PEI), all at ambient temperature, and allowed to equilibrate before the transfection mixes were added to the cells. Post-transfection bioreactor setpoint shifts were performed 2 hours post-transfection, and cells were harvested 72 hours post-transfection. Harvested cells were lysed as described in Grieger et al. (2016) Molecular Therapy 24(2): 287-297. Crude lysate samples were collected following centrifugation to remove cellular debris. Productivity was measured by vector genome titer and capsid yield. Vector genome (VG) titer was determined by droplet digital PCR (ddPCR) by standard methods using primer/probe sets specific to the transgene payload of the vector comprising the transgene. Capsid yield was determined using enzyme-linked immunosorbent assays (ELISAs) by standard methods with an immobilized antibody directed against an epitope of the capsid as encoded by the vector comprising the capsid sequences. Percentage of intact vector genomes (% Full capsids) was calculated by dividing the vector genome productivity determined by ddPCR by the number of capsids determined by ELISA.

A screen to identify post-transfection parameters of interest was performed to determine if any had a significant impact on VG titer and capsid yield. Parameters tested were temperature, pH, dissolved oxygen (DO), and power per unit volume (P/V). The post-transfection bioreactor setpoints used are described in Table 2, which also contains the resulting productivity values.

TABLE 2
Post-transfection bioreactor setpoints and productivity results

| Parameter | Temp. | pH | DO | P/V | VG titer | Capsid yield | % Full |
|---|---|---|---|---|---|---|---|
| 1 | 38 | 7 | 50 | 20 | 5.83 | 4.37 | 13 |
| 2 | 38 | 6.8 | 50 | 50 | 7.36 | 4.47 | 16 |
| 3 | 38 | 6.8 | 20 | 50 | 6.86 | 4.78 | 14 |
| 4 | 36 | 6.8 | 20 | 20 | 6.22 | 3.71 | 17 |
| 5 | 36 | 7 | 20 | 50 | 4.10 | 3.28 | 12 |
| 6 | 38 | 7 | 20 | 20 | 6.28 | 5.30 | 12 |
| 7 | 36 | 7 | 50 | 50 | 3.79 | 2.97 | 13 |
| 8 | 36 | 6.8 | 50 | 20 | 4.97 | 4.13 | 12 |

Temp.: temperature (° C.)
DO: dissolved oxygen (%)
P/V: power per unit volume (W/m³)
VG titer: vector genome titer as determined by ddPCR (E13 vg/L)
Capsid yield: capsid yield as determined by ELISA (E14 capsids/L)
% Full: percentage of intact vector genomes

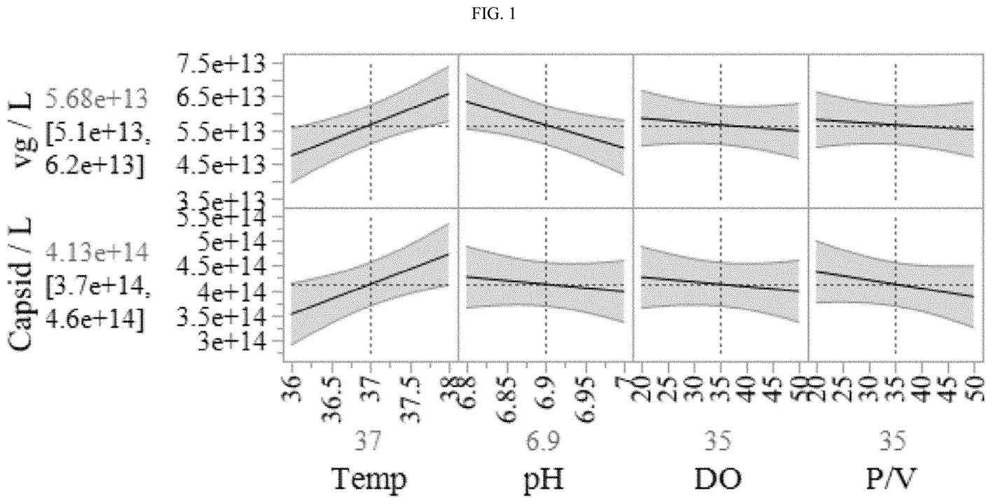

As shown in Table 2, it was found that both temperature and pH significantly affected both VG titer and capsid yield. Temperature shared a linear relationship with both VG titer and capsid yield—as temperature increased, so did VG titer and capsid yield. pH shared an inverse relationship with VG titer in that as pH increased, VG titer decreased. These observations were consistent with a relationship between tested factors and VG titer (vg/L) and capsid yield (capsids/L,) (FIG. 1). As shown in FIG. 1, as temperature increases, VG titer and capsid yield increases; and as pH increases, VG titer and capsid yield decreases.

The foregoing results identified temperature and pH as active parameters that influence AAV productivity. These parameters were further tested for possible second order relationships and interaction effects. Bioreactor setpoints used for further testing are described in Table 3, which also contains the resulting productivity values.

TABLE 3
Post-transfection bioreactor setpoints and productivity results

| Parameter | Temp. | pH | DO | VG titer | Capsid yield | % Full |
|---|---|---|---|---|---|---|
| 1 | 37 | 6.7 | 35 | 1.33E14 | 5.65 | 23 |
| 2 | 37 | 6.9 | 35 | 5.06E13 | 3.41 | 15 |
| 3 | 37 | 6.9 | 20 | 4.41E13 | 2.49 | 18 |
| 4 | 35 | 6.7 | 20 | 8.01E13 | 1.95 | 41 |
| 5 | 37 | 7.1 | 35 | 3.37E13 | 1.81 | 19 |
| 6 | 35 | 6.7 | 50 | 3.75E13 | 2.13 | 18 |
| 7 | 39 | 7.1 | 50 | 7.06E13 | 4.03 | 18 |
| 8 | 39 | 6.7 | 50 | 1.04E14 | 5.49 | 19 |
| 9 | 39 | 6.7 | 20 | 1.25E14 | 8.01 | 16 |
| 10 | 37 | 6.9 | 35 | 6.69E13 | 4.03 | 17 |
| 11 | 35 | 7.1 | 20 | 3.33E13 | 2.27 | 15 |
| 12 | 35 | 7.1 | 50 | 3.18E13 | 2.10 | 15 |
| 13 | 39 | 7.1 | 20 | 7.59E13 | 6.81 | 11 |
| 14 | 37 | 6.9 | 50 | 5.43E13 | 3.54 | 15 |
| 15 | 39 | 6.9 | 35 | 8.13E13 | 6.33 | 13 |
| 16 | 35 | 6.9 | 35 | 3.85E13 | 2.76 | 14 |

Temp.: temperature (° C.)
DO: dissolved oxygen (%)
VG titer: vector genome titer as determined by ddPCR (vg/L)
Capsid yield: capsid yield as determined by ELISA (E14 capsids/L)
% Full: percentage of intact vector genomes

As shown in Table 3, the results confirmed the significance of temperature and pH. Temperature actively affected VG titer and capsid yield, and pH actively affected VG titer but not capsid yield.

Bioreactor setpoints used to test the outer ranges of temperature and pH and their effect on productivity are described in Table 4, which also contains the resulting productivity values.

TABLE 4
Post-transfection bioreactor setpoints and productivity results

| Parameter | Temp. | pH | VG titer | Capsid yield | % Full |
|---|---|---|---|---|---|
| 1 | 41 | 6.4 | 4.95E12 | 4.78E13 | 10 |
| 2 | 39 | 6.4 | 1.26E13 | 6.66E13 | 19 |
| 3 | 41 | 6.8 | 7.80E13 | 6.42E14 | 12 |
| 4 | 39 | 6.8 | 9.16E13 | 6.94E14 | 13 |
| 5 | 40 | 6.6 | 8.20E13 | 6.83E14 | 12 |
| 6 | 41 | 6.6 | 4.83E13 | 3.40E14 | 14 |
| 7 | 40 | 6.8 | 8.63E13 | 6.24E14 | 14 |
| 8 | 39 | 6.8 | 9.25E13 | 6.69E14 | 14 |

Temp.: temperature (° C.)
VG titer: vector genome titer as determined by ddPCR (vg/L)
Capsid yield: capsid yield as determined by ELISA (capsids/L)

As shown in Table 4, it was found that increasing the temperature above 39° C. had no significant effect on VG titer and capsid yield. Decreasing the pH to below 6.7 had a significant negative effect on VG titer and capsid yield.

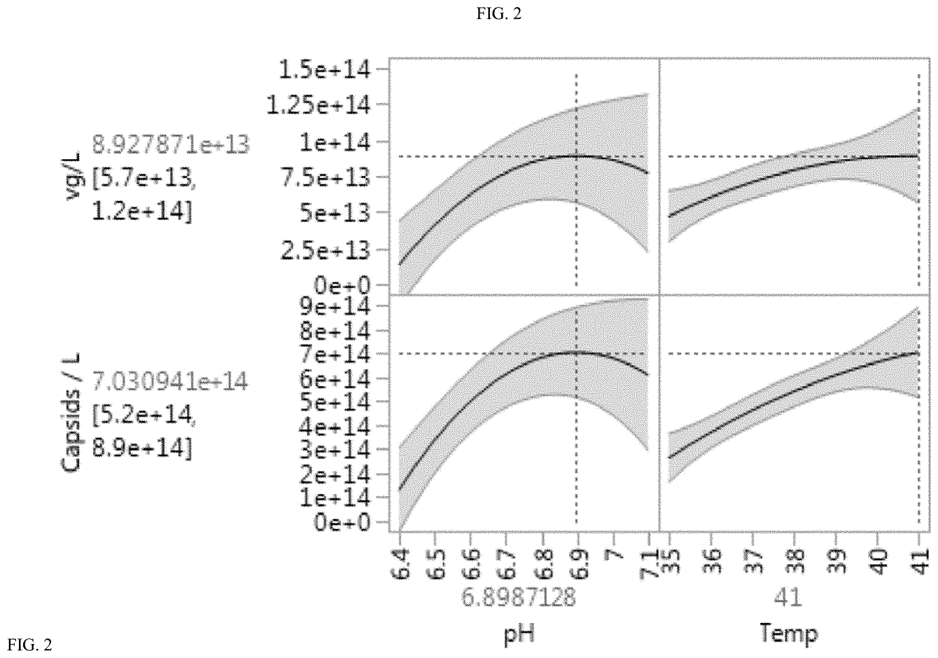

Based on the foregoing, the predicted relationship between pH and temperature on VG titer and capsid yield is consistent with 39° C. and pH 6.8 to 7 being an optimal temperature and pH, respectively, that will significantly increase VG titer and/or capsid yield (FIG. 2).

### Example 2: Optimization of Upstream Process: Post-Transfection Conditions

Based on the findings in Example 1, the effects of post-transfection temperature and pH were further tested using a second representative AAV particle. Experiments were performed according to the methods described in Example 1.

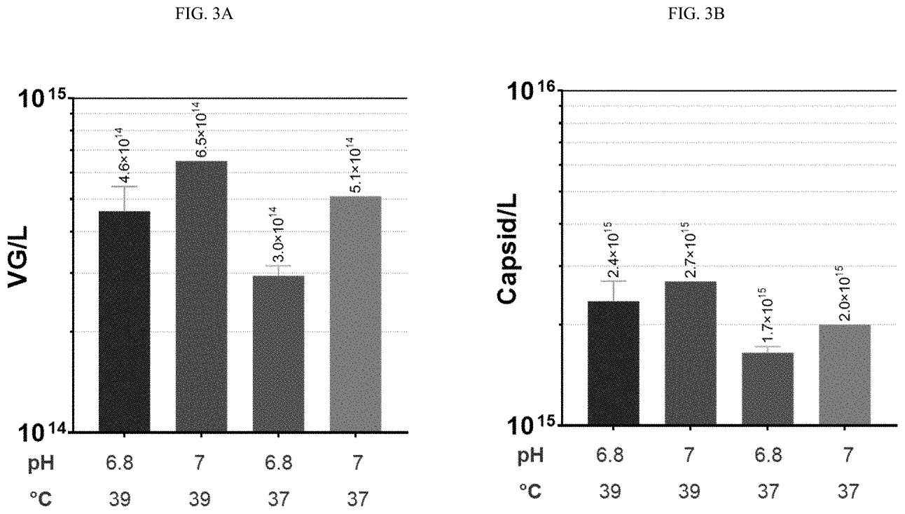

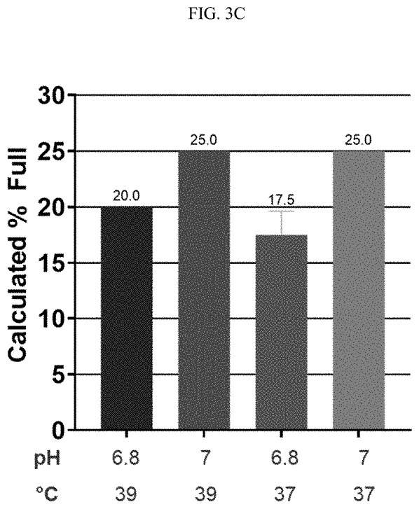

As shown in FIGS. 3A-3C, an increase in pH from 6.8 to 7 resulted in increased VG titer (FIG. 3A), capsid yield (FIG. 3B), and calculated percent intact vector genomes (Calculated % Full; FIG. 3C), at both 37° C. and 39° C. These results further suggest that a post-transfection temperature of 39° C. and pH of 6.8 to 7 are the optimal temperature and pH for maximizing AAV productivity in the systems tested.

### Example 3: Optimization of Upstream Process: Media Additives

In order to investigate the impact of upstream culture media additives on the productivity of a representative AAV particle, various additives were supplemented into the culture process and their impact on AAV productivity was assessed. Upstream transfection and lysis procedures were performed as described in Example 1 using shake flasks with the supplementation of a media additive to the culture media.

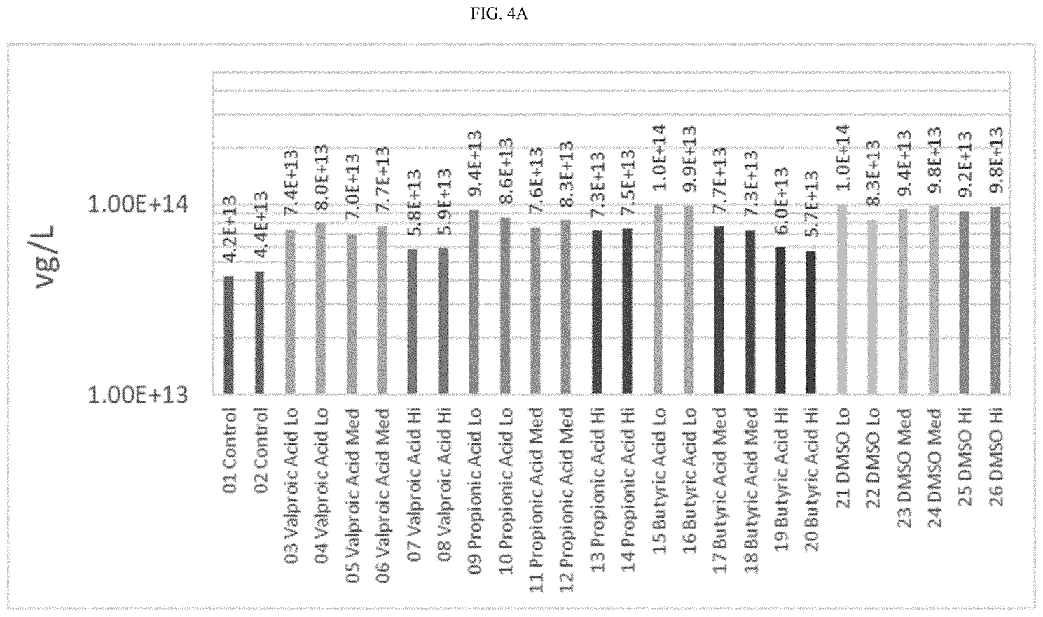

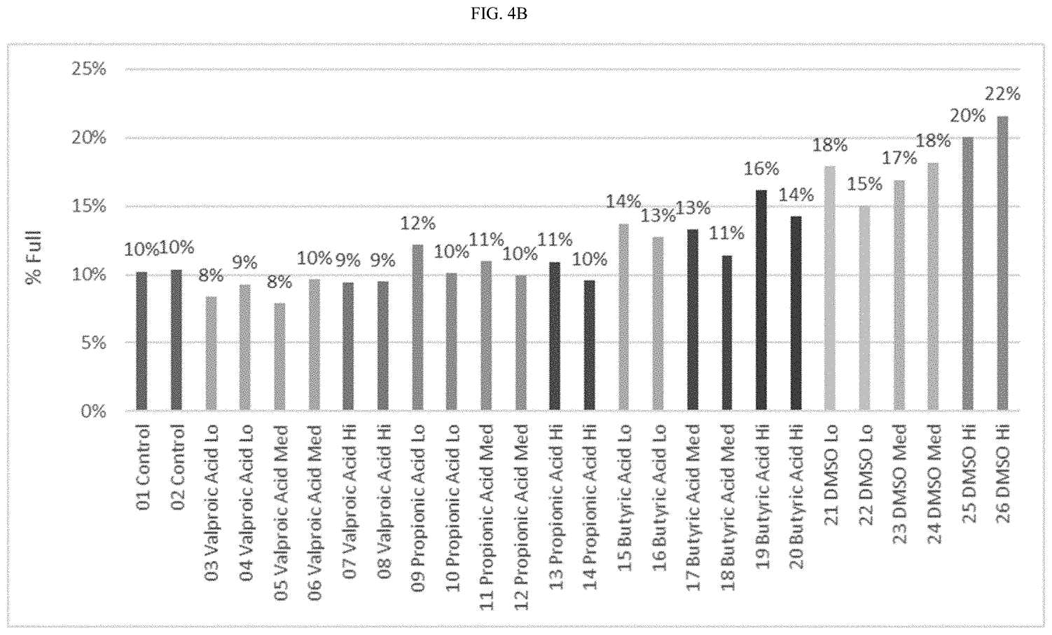

Valproic acid, propionic acid, butyric acid, and dimethyl sulfoxide (DMSO) were each tested at various concentrations (Table 5), to investigate their effect on AAV productivity when added to the culture media two hours post-transfection (FIGS. 4A and 4B).

TABLE 5
Concentration of transfection media additives

| Additive | Low (“Lo”) | | Medium (“Med”) | | High (“Hi”) | |
|---|---|---|---|---|---|---|
| Valproic acid | 2.5 | mM | 5 | mM | 7.5 | mM |
| Propionic acid | 5 | mM | 10 | mM | 15 | mM |
| Butyric acid | 2.5 | mM | 5 | mM | 7.5 | mM |
| DMSO | 0.5% | (v/v) | 0.75% | (v/v) | 1.5% | (v/v) |

AAV productivity in terms of vector genome (VG) titer, capsid yield, and percent intact vector genomes (% Full capsids) was measured as described in Example 1. It was found that the supplementation of each additive at all tested concentrations exhibited a positive increase in VG titer (FIG. 1A) and percent full capsids (FIG. 1B) over control conditions in which no additive was supplemented. As shown in FIGS. 1A and 1i, it was found that supplementing the culture media two hours post-transfection with DMSO resulted in higher percent full capsids obtained as compared to the other additives, and a general increase in VG titer.

### Example 4: Optimization of Upstream Process: DMSO Time Course Analysis

Upstream transfection and lysis procedures were performed as described in Example 1, but with the supplementation of various media additives to the culture media. Based on the finding that supplementing the culture media with DMSO resulted in improved AAV productivity (Example 3), a time course analysis was performed to investigate the effect of DMSO on productivity when DMSO was added at various time points in the process. The time course analysis was performed with 1.5% (v/v) DMSO added at 2 hours pre-transfection (Pre TFX), at the time of transfection (At TFX), and 30 minutes, 1 hours, 1.5 hours, 2 hours, or 4 hours post-transfection (Post TFX). Each experiment was performed in duplicate.

AAV productivity, in terms of vector genome (VG) titer (FIG. 5A), capsid yield (FIG. 5B), and percent intact vector genomes (Calculated % Full; FIG. 5C), was measured as described in Example 1. As shown in FIGS. 5A-5C, it was found that supplementing the culture media with DMSO two hours pre-transfection and up to 30 minutes post-transfection resulted in the higher percent yield of full capsids, as compared to the other time points tested.

### Example 5: Temperature and pH Analysis in Different AAV Particles

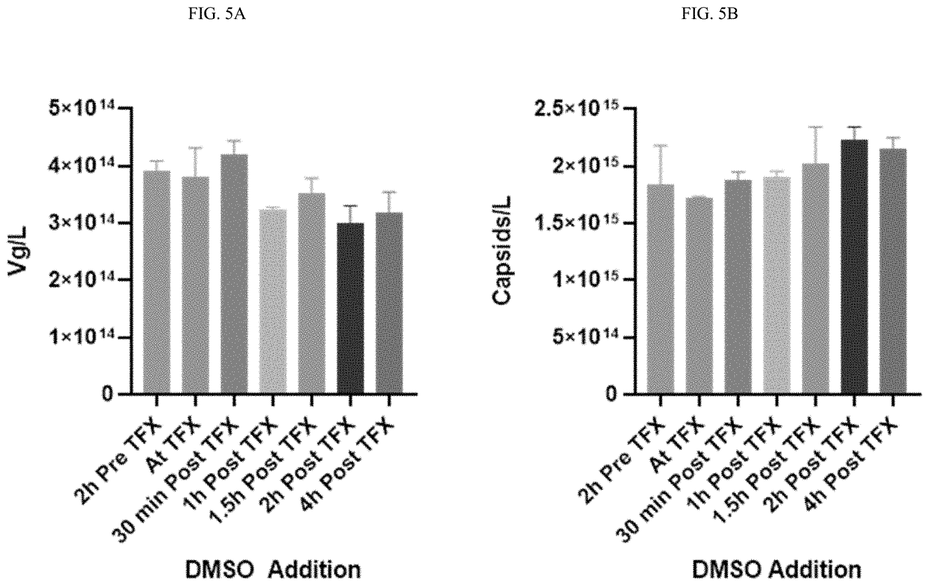

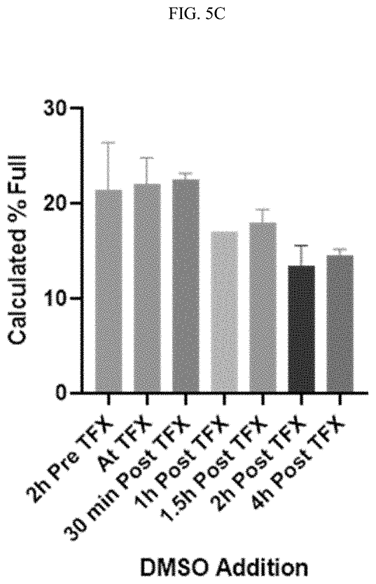

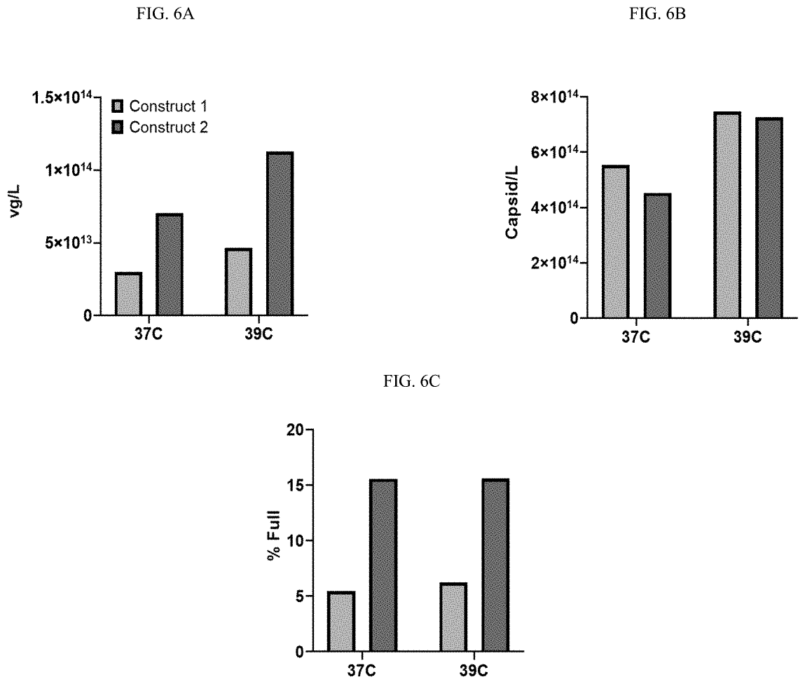

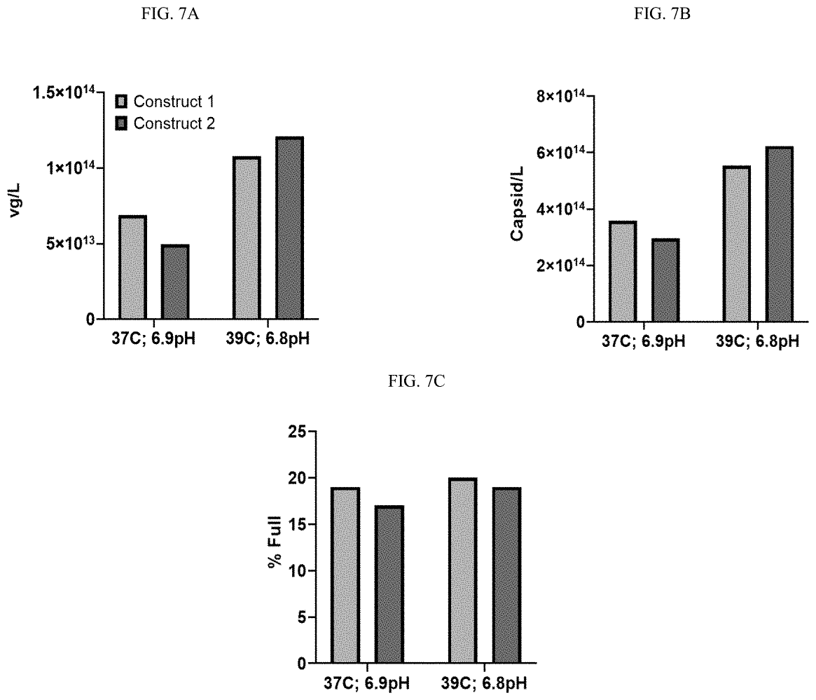

Based on the findings in Examples 1 and 2, the effects of post-transfection temperature and pH were further tested using different representative AAV particles (“Construct 1” and “Construct 2”). Experiments were performed with HEK293 cells according to the methods described in Example 1 using 125 mL shake flasks (FIG. 6) and 3 L single-use bioreactors (FIG. 7). The setpoints for the single-use bioreactor growth and lysis phases are set forth in Table 6.

TABLE 6
Bioreactor growth and lysis setpoints

| Parameter | Full fill growth | Lysis |
|---|---|---|
| Temperature (° C.) | 37 | 37 |
| Agitation (RPM) | 200 | 255 |
| P/V (W/m³) | 14.6 | 28.4 |
| pH | 7.15 ± 0.15 | Off |
| Dissolved oxygen (DO; %) | 30 | Off |

AAV productivity, in terms of vector genome (VG) titer (FIGS. 6A and 7A), capsid yield (FIGS. 6B and 7B), and percent intact vector genomes (% Full; FIGS. 6C and 7C), was measured as described in Example 1.

As shown in FIGS. 6A-6C, an increase in temperature from 37° C. to 39° C. resulted in increased VG titer (FIG. 6A), capsid yield (FIG. 6B), and comparable percent intact vector genomes (FIG. 6C). As shown in FIGS. 7A-7C, an increase in temperature from 37° C. to 39° C. and a decrease in pH from 6.9 to 6.8 resulted in increased VG titer (FIG. 7A) and capsid yield (FIG. 7B), and comparable percent intact vector genomes (FIG. 7C).

These results further suggest that a post-transfection temperature of 39° C. and pH of 6.8 to 7 are the optimal temperature and pH for maximizing AAV productivity. These results also demonstrate that these conditions can be applied to a range of AAV particles for maximizing AAV productivity.

### Example 6: Temperature, pH and DMSO Analysis in Different AAV Particles

Based on the findings above, the effects of post-transfection pH, and the effect of supplementing the culture media with DMSO at 30-120 minutes post-transfection were further tested using a different representative AAV particle. Experiments were performed with a second HEK293 cell line according to the methods described in Examples 1 and 5 using 3 L single-use bioreactors.

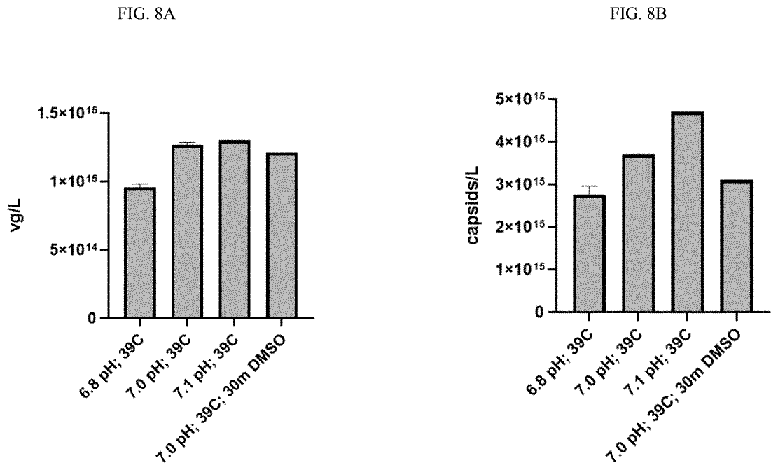

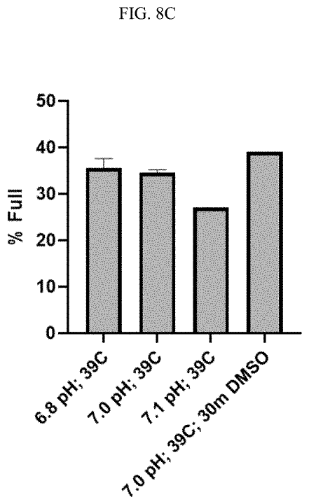

AAV productivity, in terms of vector genome (VG) titer (FIG. 8A), capsid yield (FIG. 8B), and percent intact vector genomes (% Full; FIG. 8C), was measured as described in Example 1.

As shown in FIGS. 8A-8C, an increase in pH from 6.8 to 7.0 and 7.1 at 39° C. resulted in an increased VG titer (FIG. 8A) and capsid yield (FIG. 8B). pH values of 6.8-7.1 produced similar yields of intact vector genomes (FIG. 8C). The supplementation of DMSO 120 minutes post-transfection and 30 minutes post-transfection (“30m DMSO”) at pH 7.0 produced similar VG titers (FIG. 8A), capsid yields (FIB. 8B) and intact vector genomes (FIG. 8C). These results suggest that a post-transfection temperature of 39° C. and pH of 7.0 to 7.1, and supplementing DMSO 30-120 minutes post-transfection, are the optimal conditions for maximizing AAV productivity. These results also demonstrate that these conditions can be applied to a range of AAV particles for maximizing AAV productivity.

### Example 7: DMSO Analysis in Different AAV Particles

The conditions of DMSO supplementation were further tested using different representative AAV particles. Experiments were performed with a second HEK293 cell line according to the methods described in Example 1 using shake flasks.

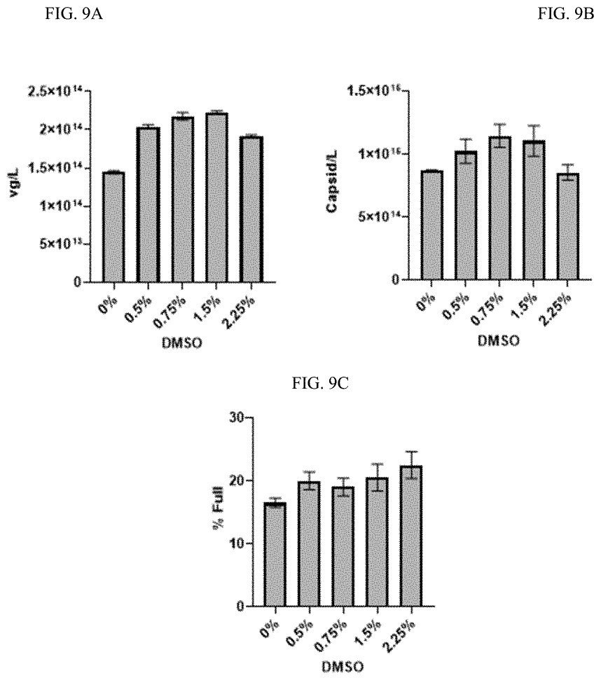

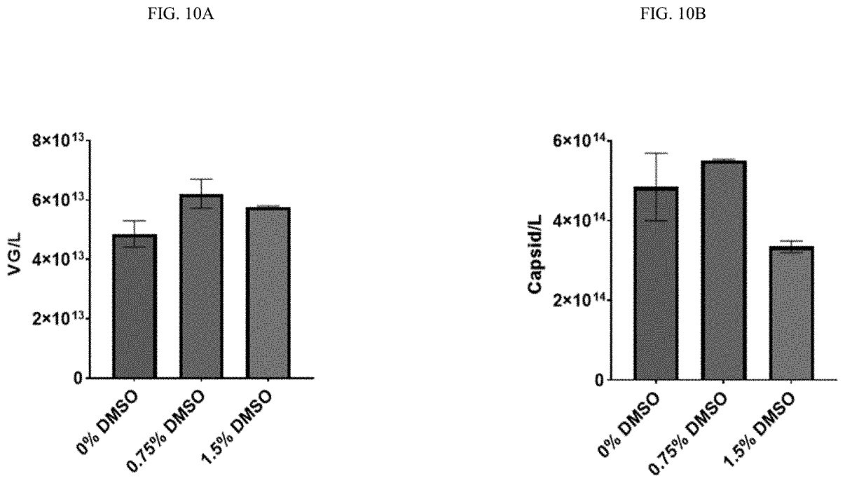

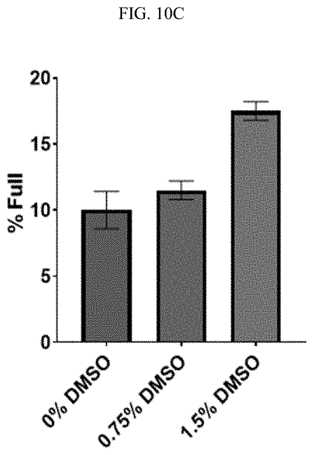

AAV productivity, in terms of vector genome (VG) titer (FIGS. 9A and 10A), capsid yield (FIGS. 9B and 10B), and percent intact vector genomes (% Full; FIGS. 9C and 10C), was measured as described in Example 1.

As shown in FIGS. 9A-9C, the supplementation of 0.5% to 2.25% DMSO two hours post-transfection resulted in increased VG titer (FIG. 9A) and increased or comparable capsid titer (FIG. 9B) up to a DMSO concentration of 1.5%. The supplementation of 0.5% to 2.25% DMSO resulted in increased percent full capsids (FIG. 9C), with the supplementation of 0.5%, 0.75%, 1.5% and 2.25% DMSO two hours post-transfection resulting in comparable percent full capsids. These results suggest that supplementation of DMSO at a concentration of 0.5% to 2.25% two hours post-transfection maximizes AAV productivity.

As shown in FIGS. 10A-10C, the supplementation of 0.75% DMSO two hours post-transfection resulted in increased VG titer (FIG. 10A), increased capsid titer (FIG. 10B) and comparable percent full capsids (FIG. 10C) compared with 0% DMSO. The supplementation of 1.5% DMSO two hours post-transfection resulted in comparable VG titer (FIG. 10A) and decreased capsid titer (FIG. 10B), but increased percent full capsids (FIG. 10C) compared with 0.75% DMSO.

These results further suggest that DMSO supplementation two hours post-transfection maximizes AAV productivity and demonstrate that these conditions can be applied to a range of AAV particles for maximizing AAV productivity.

Further embodiments of the invention are set out in the following clauses:

Clause 1. A method for producing recombinant AAV (rAAV) particles, comprising: (a) introducing into a mammalian cell a first polynucleotide comprising an rAAV genome, to generate an AAV producer cell; (b) culturing the AAV producer cell in a first culture medium at a first temperature for a first period of time; (c) culturing the AAV producer cell in a second culture medium at a second temperature for a second period of time, wherein the second temperature is about 38° C. to about 42° C., such that rAAV particles are produced by the AAV producer cell.

Clause 2. The method of clause 1, wherein the second culture medium comprises an additive selected from the group consisting of dimethyl sulfoxide (DMSO), valproic acid or a salt thereof, propionic acid or a salt thereof, and butyric acid or a salt thereof.

Clause 3. The method of clause 2, wherein the additive is DMSO.

Clause 4. The method of clause 2, wherein the additive is valproic acid or a salt thereof.

Clause 5. The method of clause 2, wherein the additive is propionic acid or a salt thereof.

Clause 6. The method of clause 2, wherein the additive is butyric acid or a salt thereof.

Clause 7. The method of any one of clauses 1-6, wherein the first temperature is about 30° C. to about 37° C., optionally about 37° C.

Clause 8. The method of any one of clauses 1-6, wherein the first temperature is about 38° C. to about 42° C., optionally about 39° C.

Clause 9. The method of any one of clauses 1-6, wherein: the first temperature is about 30° C. to about 37° C., optionally about 37° C.; and wherein the second temperature is about 38° C. to about 42° C., optionally about 39° C.

Clause 10. The method of any one of clauses 1-6, wherein: the first temperature is about 38° C. to about 42° C., optionally about 39° C.; and wherein the second temperature is about 38° C. to about 42° C., optionally about 39° C.

Clause 11. The method of any one of clauses 1-10, wherein the first and/or second culture medium has a pH of about 7.2.

Clause 12. The method of any one of clauses 1-10, wherein the first and/or second culture medium has a pH of about 6.8.

Clause 13. The method of any one of clauses 1-10, wherein the first and/or second culture medium has a pH of about 7.

Clause 14. The method of any one of clauses 1-10, wherein: the first culture medium has a pH of about 7.2; and the second culture medium has a pH of about 6.8.

Clause 15. The method of any one of clauses 1-10, wherein: the first culture medium has a pH of about 7.2; and the second culture medium has a pH of about 7.

Clause 16. The method of any one of clauses 1-10, wherein: the first culture medium has a pH of about 6.8; and the second culture medium has a pH of about 7.2.

Clause 17. The method of any one of clauses 1-10, wherein: the first culture medium has a pH of about 6.8; and the second culture medium has a pH of about 7.

Clause 18. The method of any one of the preceding clauses, wherein the second culture medium comprises about 0.1% (v/v) to about 5% (v/v) DMSO, optionally 0.5% (v/v) to about 3% (v/v) DMSO, optionally about 1.5% (v/v) DMSO.

Clause 19. The method of any one of the preceding clauses, wherein the first culture medium comprises DMSO, optionally about 0.1% (v/v) to about 5% (v/v) DMSO, optionally about 0.5% (v/v) to about 3% (v/v) DMSO, optionally about 1.5% (v/v) DMSO.

Clause 20. The method of any one of the preceding clauses, wherein the first and second culture medium comprise the same concentration of DMSO, optionally about 0.1% (v/v) to about 5% (v/v) DMSO, optionally about 0.5% (v/v) to about 3% (v/v) DMSO, optionally about 1.5% (v/v) DMSO.

Clause 21. The method of any one of clauses 1-17, wherein the second culture medium comprises about 1 mM to about 10 mM valproic acid, optionally 2.5 mM to about 7.5 mM valproic acid.

Clause 22. The method of any one of clauses 1-17, wherein the first culture medium comprises about 1 mM to about 10 mM valproic acid, optionally 2.5 mM to about 7.5 mM valproic acid.

Clause 23. The method of any one of clauses 1-17, wherein the first and second culture medium comprise the same concentration of valproic acid, optionally about 1 mM to about 10 mM valproic acid, optionally 2.5 mM to about 7.5 mM valproic acid.

Clause 24. The method of any one of clauses 1-17, wherein the second culture medium comprises about 1 mM to about 20 mM propionic acid, optionally 5 mM to about 15 mM propionic acid.

Clause 25. The method of any one of clauses 1-17, wherein the first culture medium comprises about 1 mM to about 20 mM propionic acid, optionally 5 mM to about 15 mM propionic acid.

Clause 26. The method of any one of clauses 1-17, wherein the first and second culture medium comprise the same concentration of propionic acid, optionally about 1 mM to about 20 mM propionic acid, optionally 5 mM to about 15 mM propionic acid.

Clause 27. The method of any one of clauses 1-17, wherein the second culture medium comprises about 1 mM to about 10 mM butyric acid, optionally 2.5 mM to about 7.5 mM butyric acid.

Clause 28. The method of any one of clauses 1-17, wherein the first culture medium comprises about 1 mM to about 10 mM butyric acid, optionally 2.5 mM to about 7.5 mM butyric acid.

Clause 29. The method of any one of clauses 1-17, wherein the first and second culture medium comprise the same concentration of butyric acid, optionally about 1 mM to about 10 mM butyric acid, optionally 2.5 mM to about 7.5 mM butyric acid.

Clause 30. The method of any preceding clause, wherein prior to introduction of the first polynucleotide into the mammalian cell, the mammalian cell is cultured in a third culture medium at a third temperature for a third period of time.

Clause 31. The method of clause 30, wherein the third culture medium comprises an additive selected from the group consisting of dimethyl sulfoxide (DMSO), valproic acid or a salt thereof, propionic acid or a salt thereof, and butyric acid or a salt thereof.

Clause 32. The method of clause 30 or 31, wherein the third culture medium comprises about 0.1% (v/v) to about 5% (v/v) DMSO, optionally about 0.5% (v/v) to about 3% (v/v) DMSO, optionally about 1.5% (v/v) DMSO.

Clause 33. The method of any one of clauses 30-32, wherein the first, second, and third culture medium comprise the same concentration of DMSO, optionally about 0.1% (v/v) to about 5% (v/v) DMSO, optionally about 0.5% (v/v) to about 3% (v/v) DMSO, optionally about 1.5% (v/v) DMSO.

Clause 34. The method of any one of clauses 30-33, wherein the third temperature is about 30° C. to about 37° C., optionally about 37° C.

Clause 35. The method of any one of clauses 30-33, wherein the third period of time is about 0.5 to about 3 hours.

Clause 36. The method of any one of clauses 30-33, wherein the third period of time is about 0.5 hours, about 1 hour, about 1.5 hours, or about 2 hours.

Clause 37. The method of any one of the preceding clauses, wherein the first period of time is about 0 to about 5 hours.

Clause 38. The method of clause 37, wherein the first period of time is about 0.5 hours, about 1 hour, about 1.5 hours, or about 2 hours.

Clause 39. The method of clause 37, wherein the first period of time is about 2 hours.

Clause 40. The method of clause 37, wherein the first period of time is about 0.5 hours.

Clause 41. The method of any one of the preceding clauses, wherein the second period of time is about 1 to about 100 hours.

Clause 42. The method of any one of the preceding clauses, wherein the second period of time is about 48 to about 75 hours, optionally about 65 to about 75 hours.

Clause 43. A method for producing recombinant AAV (rAAV) particles, comprising: (a) culturing a mammalian cell in a culture medium comprising about 0.1% (v/v) to about 5% (v/v) DMSO, optionally 1.5% (v/v) DMSO, at a temperature of about 30° C. to about 37° C., optionally 37° C., for about 0.5 to about 3 hours, optionally 2 hours; (b) introducing into the mammalian cell a first polynucleotide comprising an rAAV genome, to generate an AAV producer cell; (c) culturing the AAV producer cell in culture medium comprising about 0.1% (v/v) to about 5% (v/v) DMSO, optionally 1.5% (v/v) DMSO, at a temperature of about 30° C. to about 37° C., optionally 37° C., for about 0.5 to about 2 hours; and (d) culturing the AAV producer cell in a culture medium comprising about 0.1% (v/v) to about 5% (v/v) DMSO, optionally 1.5% (v/v) DMSO, at a temperature of about 38° C. to about 42° C., optionally 39° C., for about 48 to about 75 hours, optionally 70 hours, such that rAAV particles are produced by the AAV producer cell.

Clause 44. The method of clause 43, wherein step (c) is cultured for about 0.5 hours.

Clause 45. The method of clause 43, wherein step (c) is cultured for about 1 hour.

Clause 46. The method of clause 43, wherein step (c) is cultured for about 1.5 hours.

Clause 47. The method of clause 43, wherein step (c) is cultured for about 2 hours.

Clause 48. The method of any one of the preceding clauses, wherein the mammalian cell is a mammalian cell selected from the group consisting of a COS cell, a CHO cell, a BHK cell, an MDCK cell, an HEK293 cell, an HEK293T cell, a HeLa cells, an NS0 cell, a PER.C6 cell, a VERO cell, a CRL7030 cell, an HsS78Bst cell, a HeLa cell, an NIH 3T3 cell, a HepG2 cell, an SP210 cell, an R1.1 cell, a B-W cell, an L-M cell, a BSC1 cell, a BSC40 cell, a YB/20 cell, and a BMT10 cell, optionally a cell that can be grown in suspension culture, optionally an HEK293 cell or an HEK293T cell that can be grown in suspension culture, optionally HEK293F.

Clause 49. The method of any one of the preceding clauses, wherein a second polynucleotide encoding an AAV capsid protein, a third polynucleotide encoding an AAV Rep protein, and/or a fourth polynucleotide encoding one or more helper virus genes is introduced into the mammalian cell together with the first polynucleotide.

Clause 50. The method of any one of the preceding clauses, wherein a second polynucleotide encoding an AAV capsid protein, a third polynucleotide encoding an AAV Rep protein, and a fourth polynucleotide encoding one or more helper genes are introduced into the mammalian cell together with the first polynucleotide.

Clause 51. The method of clause 49 or 50, wherein the first, second, and/or third polynucleotide is comprised within a nucleic acid vector.

Clause 52. The method of clause 49 or 50, wherein the first and second polynucleotide are comprised within the same nucleic acid vector.

Clause 53. The method of clause 49 or 50, wherein the first, second, and third polynucleotide are comprised within the same nucleic acid vector.

Clause 54. The method of clause 49 or 50, wherein the first, second, third, and fourth polynucleotide are comprised within the same nucleic acid vector.

Clause 55. The method of clause 49 or 50, wherein the second, third, and fourth polynucleotide are comprised within the same nucleic acid vector.

Clause 56. The method of any one of clauses 51-55, wherein the nucleic acid vector is a plasmid or a minimal DNA vector.

Clause 57. The method of any one of the preceding clauses, wherein the polynucleotide(s) or nucleic acid vector(s) are introduced into the mammalian cell in step (a) by transfection; optionally wherein the transfection is mediated by a cationic polymer, optionally polyethylenimine.

Clause 58. The method of any one of the preceding clauses, further comprising purifying and formulating the AAV particles for administration to a human subject.

Clause 59. The method of any one of the preceding clauses, wherein the AAV is a recombinant AAV (rAAV) comprising an rAAV genome.

Clause 60. The method of clause 59, wherein the rAAV genome comprises a transgene encoding a polypeptide, miRNA, shRNA, siRNA, antisense RNA, gRNA, antagomir, miRNA sponge, RNA aptazyme, RNA aptamer, lncRNA, ribozyme, or mRNA.

Clause 61. The method of clause 59, wherein the rAAV genome comprises a transgene encoding a protein selected from the group consisting of phenylalanine hydroxylase (PAH), glucose-6-phosphatase (G6Pase), iduronate-2-sulfatase (12S), arylsulfatase A (ARSA), frataxin (FXN), and an antibody having specificity for complement component 5 (C5).

Clause 62. The method of clause 59, wherein the rAAV genome comprises a nucleotide sequence that is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 50, 51, 52, 53, or 54.

Clause 63. The method of any one of clauses 59-62, wherein the rAAV genome further comprises a 5′ inverted terminal repeat (5′ ITR) nucleotide sequence 5′ of the transgene, and a 3′ inverted terminal repeat (3′ ITR) nucleotide sequence 3′ of the transgene.

Clause 64. The method of clause 63, wherein the 5′ ITR nucleotide sequence is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 39, 41, or 42, and/or the 3′ ITR nucleotide sequence is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 40, 43, or 44.

Clause 65. The method of clause 59, wherein the rAAV genome comprises a nucleotide sequence that is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 55, 56, 57, 58, or 59.

Clause 66. The method of any one of clauses 59-65, wherein the rAAV comprises an AAV capsid comprising an AAV capsid protein.

Clause 67. The method of clause 66, wherein the AAV capsid protein is selected from the group consisting of AAV1, AAV2, AAV3, AAV4, AAV5, AAV6, AAV7, AAV8, AAV9, AAV-DJ, AAV-LK03, NP59, VOY101, VOY201, VOY701, VOY801, VOY1101, AAVPHP.N, AAVPHP.A, AAVPHP.B, PHP.B2, PHP.B3, G2A3, G2B4, G2B5, and PHP.S.

Clause 68. The method of clause 66, wherein the AAV capsid protein comprises an amino acid sequence that is at least 85% identical to the amino acid sequence of amino acids 203-736 of SEQ ID NO: 1, 2, 3, 4, 5, 6, 7, 8, 9, 10, 11, 12, 13, 15, 16, or 17.

Clause 69. The method of clause 68, wherein: the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

Clause 70. The method of clause 69, wherein: (a) the amino acid in the capsid protein corresponding to amino acid 626 of SEQ ID NO: 16 is G, and the amino acid in the capsid protein corresponding to amino acid 718 of SEQ ID NO: 16 is G; (b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (c) the amino acid in the capsid protein corresponding to amino acid 505 of SEQ ID NO: 16 is R, and the amino acid in the capsid protein corresponding to amino acid 687 of SEQ ID NO: 16 is R; (d) the amino acid in the capsid protein corresponding to amino acid 346 of SEQ ID NO: 16 is A, and the amino acid in the capsid protein corresponding to amino acid 505 of SEQ ID NO: 16 is R; or (e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

Clause 71. The method of clause 69, wherein the AAV capsid protein comprises the amino acid sequence of amino acids 203-736 of SEQ ID NO: 1, 2, 3, 4, 5, 6, 7, 8, 9, 10, 11, 12, 13, 15, 16, or 17.

Clause 72. The method of any one of clauses 66-71, wherein the AAV capsid protein comprises an amino acid sequence that is at least 85% identical to the amino acid sequence of amino acids 138-736 of SEQ ID NO: 1, 2, 3, 4, 5, 6, 7, 8, 9, 10, 11, 12, 13, 15, 16, or 17.

Clause 73. The method of clause 72, wherein: the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

Clause 74. The method of clause 73, wherein: (a) the amino acid in the capsid protein corresponding to amino acid 626 of SEQ ID NO: 16 is G, and the amino acid in the capsid protein corresponding to amino acid 718 of SEQ ID NO: 16 is G; (b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (c) the amino acid in the capsid protein corresponding to amino acid 505 of SEQ ID NO: 16 is R, and the amino acid in the capsid protein corresponding to amino acid 687 of SEQ ID NO: 16 is R; (d) the amino acid in the capsid protein corresponding to amino acid 346 of SEQ ID NO: 16 is A, and the amino acid in the capsid protein corresponding to amino acid 505 of SEQ ID NO: 16 is R; or (e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

Clause 75. The method of clause 72, wherein the AAV capsid protein comprises the amino acid sequence of amino acids 138-736 of SEQ ID NO: 1, 2, 3, 4, 5, 6, 7, 9, 10, 11, 12, 13, 15, 16, or 17.

Clause 76. The method of any one of clauses 66-75, wherein the AAV capsid protein comprises an amino acid sequence that is at least 85% identical to the amino acid sequence of amino acids 1-736 of SEQ ID NO: 1, 2, 3, 4, 5, 6, 7, 8, 9, 10, 11, 12, 13, 15, 16, or 17.

Clause 77. The method of clause 76, wherein: the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

Clause 78. The method of clause 76, wherein: (a) the amino acid in the capsid protein corresponding to amino acid 2 of SEQ ID NO: 16 is T, and the amino acid in the capsid protein corresponding to amino acid 312 of SEQ ID NO: 16 is Q; (b) the amino acid in the capsid protein corresponding to amino acid 65 of SEQ ID NO: 16 is I, and the amino acid in the capsid protein corresponding to amino acid 626 of SEQ ID NO: 16 is Y; (c) the amino acid in the capsid protein corresponding to amino acid 77 of SEQ ID NO: 16 is R, and the amino acid in the capsid protein corresponding to amino acid 690 of SEQ ID NO: 16 is K; (d) the amino acid in the capsid protein corresponding to amino acid 119 of SEQ ID NO: 16 is L, and the amino acid in the capsid protein corresponding to amino acid 468 of SEQ ID NO: 16 is S; (e) the amino acid in the capsid protein corresponding to amino acid 626 of SEQ ID NO: 16 is G, and the amino acid in the capsid protein corresponding to amino acid 718 of SEQ ID NO: 16 is G; (f) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (g) the amino acid in the capsid protein corresponding to amino acid 505 of SEQ ID NO: 16 is R, and the amino acid in the capsid protein corresponding to amino acid 687 of SEQ ID NO: 16 is R; (h) the amino acid in the capsid protein corresponding to amino acid 346 of SEQ ID NO: 16 is A, and the amino acid in the capsid protein corresponding to amino acid 505 of SEQ ID NO: 16 is R; or (i) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

Clause 79. The method of clause 76, wherein the AAV capsid protein comprises the amino acid sequence of amino acids 1-736 of SEQ ID NO: 1, 2, 3, 4, 5, 6, 7, 8, 9, 10, 11, 12, 13, 15, 16, or 17.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1-64. (canceled)

65. A method for producing recombinant AAV (rAAV) particles, comprising:
(a) introducing into a mammalian cell one or more polynucleotides comprising an rAAV genome, to generate an AAV producer cell;
(b) culturing the AAV producer cell in culture medium for a first period of time in a first set of culture conditions, wherein the first set of culture conditions comprises a first temperature; and
(c) culturing the AAV producer cell in culture medium for a second period of time in a second set of culture conditions, wherein the second set of culture conditions comprises a second temperature, wherein the second temperature is about 38° C. to about 42° C., and wherein the AAV producer cell produces rAAV particles.

66. The method of claim 65, wherein the second set of culture conditions comprises an additive selected from the group consisting of dimethyl sulfoxide (DMSO), valproic acid or a salt thereof, propionic acid or a salt thereof, and butyric acid or a salt thereof.

67. The method of claim 65, wherein the first temperature is about 30° C. to about 37° C.

68. The method of claim 65, wherein the first set of culture conditions comprises a pH of about 7.2.

69. The method of claim 66, wherein the second set of culture conditions comprises a pH of about 6.8 to about 7.

70. The method of claim 66, wherein the second set of culture conditions comprises about 0.1% (v/v) to about 5% (v/v) DMSO in the culture medium.

71. The method of claim 66, wherein the second set of culture conditions comprises about 1 mM to about 10 mM valproic acid in the culture medium.

72. The method of claim 66, wherein the second set of culture conditions comprises about 1 mM to about 20 mM propionic acid in the culture medium.

73. The method of claim 66, wherein the second set of culture conditions comprises about 1 mM to about 10 mM butyric acid in the culture medium.

74. The method of claim 65, further comprising culturing the mammalian cell, prior to introduction of one or more polynucleotides into the mammalian cell, in a culture medium for a third period of time in a third set of culture conditions, wherein the third set of culture conditions comprises a third temperature.

75. The method of claim 74, wherein the third temperature is about 30° C. to about 37° C.

76. The method of claim 74, wherein the third set of culture conditions comprises a culture time of about 0.5 to about 3 hours.

77. The method of claim 65, wherein the first period of time is about 0.5 to about 5 hours.

78. The method of claim 65, wherein the second period of time is about 1 to about 100 hours.

79. The method of claim 65, further comprising introducing into the mammalian cell prior to step (b) a second polynucleotide encoding an AAV capsid protein, a third polynucleotide encoding an AAV Rep protein, and a fourth polynucleotide encoding one or more helper virus genes.

80. The method of claim 65, further comprising purifying and formulating the AAV particles for administration to a human subject.

81. The method of claim 65, wherein the rAAV genome comprises a transgene encoding a polypeptide, miRNA, shRNA, siRNA, antisense RNA, gRNA, antagomir, miRNA sponge, RNA aptazyme, RNA aptamer, lncRNA, ribozyme, or mRNA.

82. The method of claim 81, wherein the rAAV genome further comprises a 5′ inverted terminal repeat (5′ ITR) nucleotide sequence 5′ of the transgene and a 3′ inverted terminal repeat (3′ ITR) nucleotide sequence 3′ of the transgene.

83. A method for producing recombinant AAV (rAAV) particles, comprising:
(a) introducing into a mammalian cell one or more polynucleotides comprising an rAAV genome to generate an AAV producer cell;
(b) culturing the AAV producer cell in culture medium for a first period of time in a first set of culture conditions, wherein the first set of culture conditions comprises absence of an additive selected from the group consisting of dimethyl sulfoxide (DMSO), valproic acid or a salt thereof, propionic acid or a salt thereof, and butyric acid or a salt thereof; and
(c) culturing the AAV producer cell in culture medium for a second period of time in a second set of culture conditions to produce rAAV particles, wherein the second set of culture conditions comprises presence of an additive selected from the group consisting of dimethyl sulfoxide (DMSO), valproic acid or a salt thereof, propionic acid or a salt thereof, and butyric acid or a salt thereof.

84. A method for producing recombinant AAV (rAAV) particles, comprising:
(a) introducing into a mammalian cell one or more polynucleotides comprising an rAAV genome to generate an AAV producer cell;
(b) culturing the AAV producer cell in culture medium for a first period of time in a first set of culture conditions, wherein the first set of culture conditions comprises a pH of about 7.2; and
(c) culturing the AAV producer cell in culture medium for a second period of time in a second set of culture conditions to produce rAAV particles, wherein the second set of culture conditions comprises a pH of about 6.8 to about 7.

85. A method for producing a composition of recombinant AAV (rAAV) particles comprising:
(a) culturing mammalian cells under a first set of culture conditions;
(b) introducing into the cultured mammalian cells one or more polynucleotides comprising an rAAV genome to generate AAV producer cells; and
(c) modifying the first set of culture conditions to a second set of culture conditions to produce a composition of rAAV particles, wherein the composition of AAV particles comprises at least about 1×10¹⁵ viral genomes.

86. The method of claim 85, wherein the second set of culture conditions comprises one or more of
(a) a culture medium comprising an additive selected from the group consisting of dimethyl sulfoxide (DMSO), valproic acid or a salt thereof, propionic acid or a salt thereof, and butyric acid or a salt thereof;
(b) a temperature of 38-42° C.; and
(c) a pH of about 6.8.

87. The method of claim 85, wherein the first set of culture conditions comprises one or more of
(a) a culture medium comprising the absence of an additive selected from the group consisting of dimethyl sulfoxide (DMSO), valproic acid or a salt thereof, propionic acid or a salt thereof, and butyric acid or a salt thereof;
(b) a temperature of 30-37° C.; and
(c) a pH of about 7.2.


SEQUENCE LISTING (59 sequences)

SEQ ID NO: 1 - protein, 736 aa, synthetic construct
  REGION 1..736: note = Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 736 aa, synthetic construct
  REGION 1..736: note = AAV isolate
MTADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKQLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTGGNFHPSPLMGGFGMKHPPPQILIKNTPVPADPPTAFNKDKLNSFITQYSTGQVSVEIEWELQKENSKRWNPEIQYTSNYYKSNNVEFAVNTGGVYSEPRPIGTRYLTRNL

SEQ ID NO: 4 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPINAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTYGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 5 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIDKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 6 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVLQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGSSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 7 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQARAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAVDAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 9 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPREPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASCGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDRAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKKNSKRWNPEIQYTSNYYKSNNVEFAVNTEGVYSEPRPIGTRYLTRNL

SEQ ID NO: 12 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPHDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFNVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSMEIEWELQKENSKRWNPEIQYTSNYYKSNNVEFAVNTEGVYSEPRPIGTRYLTRNL

SEQ ID NO: 13 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - DNA, 198 bp, synthetic construct
  misc_feature 1..198: note = Synthetic polynucleotide
  source 1..198: mol_type = other DNA
gatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 15 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWERQKENSKRWNPEIQYTSNYYKSNNVEFAVNTEGVYSEPRPIGTRYLTRNL

SEQ ID NO: 16 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFADSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 17 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEIAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYCKSNNVEFAVNTEGVYSEPRPIGTRYLTRNL

SEQ ID NO: 18 - DNA, 365 bp, synthetic construct
  misc_feature 1..365: note = Synthetic polynucleotide
  source 1..365: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatg

SEQ ID NO: 19 - DNA, 380 bp, synthetic construct
  misc_feature 1..380: note = Synthetic polynucleotide
  source 1..380: mol_type = other DNA
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatg

SEQ ID NO: 20 - DNA, 479 bp, synthetic construct
  misc_feature 1..479: note = Synthetic polynucleotide
  source 1..479: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggag

SEQ ID NO: 21 - DNA, 1246 bp, synthetic construct
  misc_feature 1..1246: note = Synthetic polynucleotide
  source 1..1246: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcgg

SEQ ID NO: 22 - DNA, 953 bp, synthetic construct
  misc_feature 1..953: note = Synthetic polynucleotide
  source 1..953: mol_type = other DNA
aattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaag

SEQ ID NO: 23 - DNA, 1168 bp, synthetic construct
  misc_feature 1..1168: note = Synthetic polynucleotide
  source 1..1168: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaggggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 24 - DNA, 92 bp, synthetic construct
  misc_feature 1..92: note = Synthetic polynucleotide
  source 1..92: mol_type = other DNA
aagaggtaagggtttaagggatggttggttggtggggtattaatgtttaattacctggagcacctgcctgaaatcactttttttcaggttgg

SEQ ID NO: 25 - DNA, 79 bp, synthetic construct
  misc_feature 1..79: note = Synthetic polynucleotide
  source 1..79: mol_type = other DNA
aagaggtaagggtttaagggatggttggttggtggggtattaatgtttaattacctggagcacctgcctgaaatcactt

SEQ ID NO: 26 - DNA, 95 bp, synthetic construct
  misc_feature 1..95: note = Synthetic polynucleotide
  source 1..95: mol_type = other DNA
cctctgctaaccatgttcatgccttcttctttttcctacagctcctgggcaacgtgctggttattgtgctgtctcatcattttggcaaagaattc

SEQ ID NO: 27 - DNA, 90 bp, synthetic construct
  misc_feature 1..90: note = Synthetic polynucleotide
  source 1..90: mol_type = other DNA
cctctgctaaccatgttcatgccttcttctttttcctacagctcctgggcaacgtgctggttattgtgctgtctcatcattttggcaaag

SEQ ID NO: 28 - DNA, 918 bp, synthetic construct
  misc_feature 1..918: note = Synthetic polynucleotide
  source 1..918: mol_type = other DNA
ggcatcctaaaaaatattcagtggaaacgtaaaaacattaaagactgattaaacatcgcagcatgacacagatttagcaactgagcataaataatttgactcggatactgctccaaaatccgaagaggaccaatttcttccaggaggacaactacctcgtcctctgcagacccctctcctcggcagctgaaggagtgtggccaatctgcctccacctccccgcggaccccctactctcaggacctcctgcagcaccccaaactggaagtggccgctgcagacccaaggacgaggggcacgcgggagccggcagccctagtggagcggttggagatgttgaggtgggagggtcacccaggtggggtgaggctggggtaggtagcggagtgaacggcttccgaagctctgggccgcccccaggttggactaagcaggcgctctgtcttcgcccccgcccagggtgggcgtctcctgaggactccccgccacacctgacccgagaccgcgcgcccagcctagaacgcttccccgacccagcgtagggccgccgcgactggcgggcgagggtcggcgggaggcctggcgaacccgggggcgggaccaggcgggcaaggcccggctgccgcagcgccgctctgcgcgaggcggctccgccgcggcggagggatacggcgcaccatatatatatcgcggggcgcagactcgcgctccggcagtggtgctgggagtgtcgtggacgccgtgccgttactcgtagtcaggcggcggcgcaggcggcggcggcggcatagcgcacagcgcgccttagcagcagcagcagcagcagcggcatcggaggtacccccgccgtcgcagcccccgcgctggtgcagccaccctcgctccctctgctcttcctcccttcgctcgcaccaagag

SEQ ID NO: 29 - DNA, 192 bp, synthetic construct
  misc_feature 1..192: note = Synthetic polynucleotide
  source 1..192: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 30 - DNA, 205 bp, synthetic construct
  misc_feature 1..205: note = Synthetic polynucleotide
  source 1..205: mol_type = other DNA
aatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagg

SEQ ID NO: 31 - DNA, 913 bp, synthetic construct
  misc_feature 1..913: note = Synthetic polynucleotide
  source 1..913: mol_type = other DNA
tagggaggtcctgcacagaaggggaggagggggcagcagctgtctgaccactgttggtcttgcaacttgtgtccccaggttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctctccccacccccaggattgtaactgaaatgcttcactggtgctccttttgttttaaggcattggatcttcatagctactgatcgtgcccaagcacacagtatctgcagcaaccacttaggcctccaggaatgtggtgaccattgaccctaattcattccccttcatggatcctatgtaaccatcctccaaaaagagctttcgcaaactcaaataaacacaggaaaggaagaccttcttatctttgagagtatatgtttagccctatagctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggac

SEQ ID NO: 32 - DNA, 423 bp, synthetic construct
  misc_feature 1..423: note = Synthetic polynucleotide
  source 1..423: mol_type = other DNA
gctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggac

SEQ ID NO: 33 - DNA, 592 bp, synthetic construct
  misc_feature 1..592: note = Synthetic polynucleotide
  source 1..592: mol_type = other DNA
gtaaattttatggaatgtgaatcataattcaatttttcaacatgcgttaggagggacatttcaaactcttttttaccctagactttcctaccatcacccagagtatccagccaggaggggaggggctagagacaccagaagtttagcagggaggagggcgtagggattcggggaatgaagggatgggattcagactagggccaggacccagggatggagagaaagagatgagagtggtttgggggcttggtgacttagagaacagagctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 34 - DNA, 72 bp, synthetic construct
  misc_feature 1..72: note = Synthetic polynucleotide
  source 1..72: mol_type = other DNA
gggggaggctgctggtgaatattaaccaaggtcaccccagttatcggaggagcaaacaggggctaagtccac

SEQ ID NO: 35 - DNA, 170 bp, synthetic construct
  misc_feature 1..170: note = Synthetic polynucleotide
  source 1..170: mol_type = other DNA
cgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatca

SEQ ID NO: 36 - DNA, 1873 bp, synthetic construct
  misc_feature 1..1873: note = Synthetic polynucleotide
  source 1..1873: mol_type = other DNA
gatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 37 - DNA, 1061 bp, synthetic construct
  misc_feature 1..1061: note = Synthetic polynucleotide
  source 1..1061: mol_type = other DNA
tagggaggtcctgcacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacag

SEQ ID NO: 38 - DNA, 398 bp, synthetic construct
  misc_feature 1..398: note = Synthetic polynucleotide
  source 1..398: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagg

SEQ ID NO: 39 - DNA, 145 bp, synthetic construct
  misc_feature 1..145: note = Synthetic polynucleotide
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 40 - DNA, 145 bp, synthetic construct
  misc_feature 1..145: note = Synthetic polynucleotide
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 41 - DNA, 106 bp, synthetic construct
  misc_feature 1..106: note = Synthetic polynucleotide
  source 1..106: mol_type = other DNA
ctgcgcgctcgctcgctcactgaggccgcccgggcaaagcccgggcgtcgggcgacctttggtcgcccggcctcagtgagcgagcgagcgcgcagagagggagtgg

SEQ ID NO: 42 - DNA, 167 bp, synthetic construct
  misc_feature 1..167: note = Synthetic polynucleotide
  source 1..167: mol_type = other DNA
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaagggggttttgta

SEQ ID NO: 43 - DNA, 167 bp, synthetic construct
  misc_feature 1..167: note = Synthetic polynucleotide
  source 1..167: mol_type = other DNA
tacaaaacctccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacaggggggagag

SEQ ID NO: 44 - DNA, 143 bp, synthetic construct
  misc_feature 1..143: note = Synthetic polynucleotide
  source 1..143: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 45 - DNA, 46 bp, synthetic construct
  misc_feature 1..46: note = Synthetic polynucleotide
  source 1..46: mol_type = other DNA
ggaggggtggagtcgtgacgtgaattacgtcatagggttagggagg

SEQ ID NO: 46 - DNA, 37 bp, synthetic construct
  misc_feature 1..37: note = Synthetic polynucleotide
  source 1..37: mol_type = other DNA
gtagataagtagcatggcgggttaatcattaactaca

SEQ ID NO: 47 - DNA, 122 bp, synthetic construct
  misc_feature 1..122: note = Synthetic polynucleotide
  source 1..122: mol_type = other DNA
aacttgtttattgcagcttataatggttacaaataaagcaatagcatcacaaatttcacaaataaagcatttttttcactgcattctagttgtggtttgtccaaactcatcaatgtatctta

SEQ ID NO: 48 - DNA, 133 bp, synthetic construct
  misc_feature 1..133: note = Synthetic polynucleotide
  source 1..133: mol_type = other DNA
tgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 49 - DNA, 171 bp, synthetic construct
  misc_feature 1..171: note = Synthetic polynucleotide
  source 1..171: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagg

SEQ ID NO: 50 - DNA, 4075 bp, synthetic construct
  misc_feature 1..4075: note = Synthetic polynucleotide
  source 1..4075: mol_type = other DNA
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaggaacgttttattttcccagagacaggctcagggacttctgggtgtagtggctgtgcagggcttcgtggagcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtgtggccagatcctggcaggatctcgcccatccattccaggccttggccaggggcctgccgcacccactggatccagtagttgctgaagatgtggccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggccgcggcacgtgcttaaggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatctccggagggggaggctgctggtgaatattaaccaaggtcaccccagttatcggaggagcaaacaggggctaagtccacctcgagccatggcgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 51 - DNA, 3533 bp, synthetic construct
  misc_feature 1..3533: note = Synthetic polynucleotide
  source 1..3533: mol_type = other DNA
gcttcaggagcagttgtgcgaatagctggagaacaccaggctggatttaaacccagatcgctcttacatttgctctttacctgctgtgctcagcgttcacgtgccctctagctgtagttttctgaagtcagcgcacagcaaggcagtgtgcttagaggttaacagaagggaaaacaacaacaacaaaaatctaaatgagaatcctgactgtttcagctgggggtaaggggggcggattattcatataattgttataccagacggtcgcaggcttagtccaattgcagagaactcgcttcccaggcttctgagagtcccggaagtgcctaaacctgtctaatcgacggggcttgggtggcccgtcgctccctggcttcttccctttacccagggcgggcagcgaagtggtgcctcctgcgtcccccacaccctccctcagcccctcccctccggcccgtcctgggcaggtgacctggagcatccggcaggctgccctggcctcctgcgtcaggacaacgcccacgaggggcgttactgtgcggagatgcaccacgcaagagacaccctttgtaactctcttctcctccctagtgcgaggttaaaaccttcagccccacgtgctgtttgcaaacctgcctgtacctgaggccctaaaaagccagagacctcactcccggggagccagcatgtccactgcggtcctggaaaacccaggcttgggcaggaaactctctgactttggacaggtgagccacggcagcctgagctgctcagttaggggaatttgggcctccagagaaagagatctgaagactgctggtgcttcctggtttcataagctcagtaagaagtctgaattcgttggaagctgatgagaatatccaggaagtcaacagacaaatgtcctcaacaattgtttctaagtaggagaacatctgtcctcggtggctttcacaggaaaagcttctgacctcttctcttcctcccacagggcggtaccagatctggcagcggagagggcagaggaagtcttctaacatgcggtgacgtggaggagaatcccggcccttccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaatgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaactgggatgggatgtggaatccttctagatttcttttgtaatatttataaagtgctctcagcaaggtatcaaaatggcaaaattgtgagtaactatcctcctttcattttgggaagaagatgaggcatgaagagaattcagacagaaacttactcagaccaggggaggcagaaactaagcagagaggaaaatgaccaagagttagccctgggcatggaatgtgaaagaaccctaaacgtgacttggaaataatgcccaaggtatattccattctccgggatttgttggcattttcttgaggtgaagaattgcagaatacattctttaatgtgacctacatatttacccatgggaggaagtctgctcctggactcttgagattcagtcataaagcccaggccagggaaataatgtaagtctgcaggcccctgtcatcagtaggattagggagaagagttctcagtagaaaacagggaggctggagagaaaagaatggttaatgttaacgttaatataactagaaagactgcagaacttaggactgatttttatttgaatccttaaaaaaaaaatttcttatgaaaatagtacatggctcttaggagacagaacttattgtacagaggaacagcgtgagagtcagagtgatcccagaacaggtcctggctccatcctgcacatagttttggtgctgctggcaatacggtccccacaactgtgggaaggggttaggggcagggatctcatcaggaaagcataggggtttaaagttctttatagagcacttagaagattgagaatccacaaattatattaataacaaacaaagtagtgtcgtgttatatagtaaatgtgaatttgcagacacatttagggaaaagttataattaaaaaaataggctgtatatata

SEQ ID NO: 52 - DNA, 4061 bp, synthetic construct
  misc_feature 1..4061: note = Synthetic polynucleotide
  source 1..4061: mol_type = other DNA
tgcggccgcttcaggagcagttgtgcgaatagctggagaacaccaggctggatttaaacccagatcgctcttacatttgctctttacctgctgtgctcagcgttcacgtgccctctagctgtagttttctgaagtcagcgcacagcaaggcagtgtgcttagaggttaacagaagggaaaacaacaacaacaaaaatctaaatgagaatcctgactgtttcagctgggggtaaggggggcggattattcatataattgttataccagacggtcgcaggcttagtccaattgcagagaactcgcttcccaggcttctgagagtcccggaagtgcctaaacctgtctaatcgacggggcttgggtggcccgtcgctccctggcttcttccctttacccagggcgggcagcgaagtggtgcctcctgcgtcccccacaccctccctcagcccctcccctccggcccgtcctgggcaggtgacctggagcatccggcaggctgccctggcctcctgcgtcaggacaacgcccacgaggggcgttactgtgcggagatgcaccacgcaagagacaccctttgtaactctcttctcctccctagtgcgaggttaaaaccttcagccccacgtgctgtttgcaaacctgcctgtacctgaggccctaaaaagccagagacctcactcccggggagccagcatgtccactgcggtcctggaaaacccaggcttgggcaggaaactctctgactttggacaggtgagccacggcagcctgagctgctcagttaggggaatttgggcctccagagaaagagatctgaagactgctggtgcttcctggtttcataagctcagtaagaagtctgaattcgttggaagctgatgagaatatccaggaagtcaacagacaaatgtcctcaacaattgtttctaagtaggagaacatctgtcctcggtggctttcacaggaaccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaccgccaccatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagtgtgcgttacgacccttatacccagaggatcgaggtgctggataatacacagcagctgaagatcctggctgactcaatcaatagcgaaatcggaatcctgtgctccgccctgcagaaaatcaaatgagaattcaaggcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagctttacgtacgatcgtcgactgggatgggatgtggaatccttctagatttcttttgtaatatttataaagtgctctcagcaaggtatcaaaatggcaaaattgtgagtaactatcctcctttcattttgggaagaagatgaggcatgaagagaattcagacagaaacttactcagaccaggggaggcagaaactaagcagagaggaaaatgaccaagagttagccctgggcatggaatgtgaaagaaccctaaacgtgacttggaaataatgcccaaggtatattccattctccgggatttgttggcattttcttgaggtgaagaattgcagaatacattctttaatgtgacctacatatttacccatgggaggaagtctgctcctggactcttgagattcagtcataaagcccaggccagggaaataatgtaagtctgcaggcccctgtcatcagtaggattagggagaagagttctcagtagaaaacagggaggctggagagaaaagaatggttaatgttaacgttaatataactagaaagactgcagaacttaggactgatttttatttgaatccttaaaaaaaaaatttcttatgaaaatagtacatggctcttaggagacagaacttattgtacagaggaacagcgtgagagtcagagtgatcccagaacaggtcctggctccatcctgcacatagttttggtgctgctggcaatacggtccccacaactgtgggaaggggttaggggcagggatctcatcaggaaagcataggggtttaaagttctttatagagcacttagaagattgagaatccacaaattatattaataacaaacaaagtagtgtcgtgttatatagtaaatgtgaatttgcagacacatttagggaaaagttataattaaaaaaataggctgtatatatacctgcagg

SEQ ID NO: 53 - DNA, 2141 bp, synthetic construct
  misc_feature 1..2141: note = Synthetic polynucleotide
  source 1..2141: mol_type = other DNA
gcggccgcaactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaccatgcccccacccaggaccggaagaggcctgctgtggctgggcctggtgctctcttccgtgtgcgtggccctgggaagcgaaacccaggccaacagcacaaccgacgccctgaatgtgctgctgatcattgtggacgatctgagaccctccctgggctgttacggcgacaaactggtgcggtccccaaacatcgaccagctggcctcccactccctgctgttccagaacgccttcgcccagcaggccgtgtgtgcccccagcagggtgagcttcctgaccggcagaagacctgacaccaccaggctgtacgactttaacagctactggcgggtgcacgccggcaatttcagcaccattcctcagtacttcaaggagaatggctacgtgacaatgtccgtgggcaaggtgtttcatcccggcattagctccaaccacaccgacgatagcccatactcctggtccttccccccctaccatccctccagcgagaagtacgagaacaccaaaacctgcagaggccctgacggagagctgcacgccaacctgctgtgccctgtggatgtcctggatgtgcccgaaggtaagggtttaagggatggttggttggtggggtattaatgtttaattacctggagcacctgcctgaaatcactttttttcaggcaccctgccagacaagcagtccacagagcaggccatccagctgctggagaagatgaagacaagcgccagccccttctttctggccgtgggataccacaagcctcacattccattccggtacccaaaagagttccagaagctgtaccctctggaaaacatcaccctggcccctgaccccgaggtgccagacgggctgcctcctgtggcctacaatccctggatggacatcagacagcgggaggatgtgcaggccctgaatatttccgtgccctatgggcccatccctgtggactttcagcggaaaatcagacagtcttactttgccagcgtgtcctacctggacacccaggtgggccgcctgctctcagccctggacgacctgcagctggccaattccaccatcatcgccttcaccagcgatcacggctgggccctgggcgagcacggggagtgggccaaatacagcaacttcgatgtggccacccacgtgcctctgattttttatgtgcccggccggacagccagcctgcccgaggccggggagaagctctttccttacctggaccctttcgactctgccagccagctgatggaacctggcagacagagcatggacctggtggagctggtgagcctcttccccactctggccggcctggctggcctgcaggtgccaccaagatgcccagtgccttctttccacgtggagctgtgtagagagggaaagaacctgctgaagcacttcagatttagagatctggaggaggatccctacctgccaggcaacccaagggagctgatcgcctacagccagtatcccagaccctctgatatcccccagtggaacagcgataagccctccctgaaagacatcaagattatgggctactccatcaggaccattgactaccggtacacagtgtgggtgggcttcaaccccgatgagtttctggccaacttcagcgacatccacgccggcgagctgtattttgtggactccgaccccctgcaggaccacaacatgtacaacgactcccagggcggcgacctgttccagctgctgatgccctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaacctgcaggtctagatacgta

SEQ ID NO: 54 - DNA, 2042 bp, synthetic construct
  misc_feature 1..2042: note = Synthetic polynucleotide
  source 1..2042: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaccgccaccatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaatgaatgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 55 - DNA, 3939 bp, synthetic construct
  misc_feature 1..3939: note = Synthetic polynucleotide
  source 1..3939: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcttcaggagcagttgtgcgaatagctggagaacaccaggctggatttaaacccagatcgctcttacatttgctctttacctgctgtgctcagcgttcacgtgccctctagctgtagttttctgaagtcagcgcacagcaaggcagtgtgcttagaggttaacagaagggaaaacaacaacaacaaaaatctaaatgagaatcctgactgtttcagctgggggtaaggggggcggattattcatataattgttataccagacggtcgcaggcttagtccaattgcagagaactcgcttcccaggcttctgagagtcccggaagtgcctaaacctgtctaatcgacggggcttgggtggcccgtcgctccctggcttcttccctttacccagggcgggcagcgaagtggtgcctcctgcgtcccccacaccctccctcagcccctcccctccggcccgtcctgggcaggtgacctggagcatccggcaggctgccctggcctcctgcgtcaggacaacgcccacgaggggcgttactgtgcggagatgcaccacgcaagagacaccctttgtaactctcttctcctccctagtgcgaggttaaaaccttcagccccacgtgctgtttgcaaacctgcctgtacctgaggccctaaaaagccagagacctcactcccggggagccagcatgtccactgcggtcctggaaaacccaggcttgggcaggaaactctctgactttggacaggtgagccacggcagcctgagctgctcagttaggggaatttgggcctccagagaaagagatctgaagactgctggtgcttcctggtttcataagctcagtaagaagtctgaattcgttggaagctgatgagaatatccaggaagtcaacagacaaatgtcctcaacaattgtttctaagtaggagaacatctgtcctcggtggctttcacaggaaaagcttctgacctcttctcttcctcccacagggcggtaccagatctggcagcggagagggcagaggaagtcttctaacatgcggtgacgtggaggagaatcccggcccttccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaatgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaactgggatgggatgtggaatccttctagatttcttttgtaatatttataaagtgctctcagcaaggtatcaaaatggcaaaattgtgagtaactatcctcctttcattttgggaagaagatgaggcatgaagagaattcagacagaaacttactcagaccaggggaggcagaaactaagcagagaggaaaatgaccaagagttagccctgggcatggaatgtgaaagaaccctaaacgtgacttggaaataatgcccaaggtatattccattctccgggatttgttggcattttcttgaggtgaagaattgcagaatacattctttaatgtgacctacatatttacccatgggaggaagtctgctcctggactcttgagattcagtcataaagcccaggccagggaaataatgtaagtctgcaggcccctgtcatcagtaggattagggagaagagttctcagtagaaaacagggaggctggagagaaaagaatggttaatgttaacgttaatataactagaaagactgcagaacttaggactgatttttatttgaatccttaaaaaaaaaatttcttatgaaaatagtacatggctcttaggagacagaacttattgtacagaggaacagcgtgagagtcagagtgatcccagaacaggtcctggctccatcctgcacatagttttggtgctgctggcaatacggtccccacaactgtgggaaggggttaggggcagggatctcatcaggaaagcataggggtttaaagttctttatagagcacttagaagattgagaatccacaaattatattaataacaaacaaagtagtgtcgtgttatatagtaaatgtgaatttgcagacacatttagggaaaagttataattaaaaaaataggctgtatatatacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 56 - DNA, 2356 bp, synthetic construct
  misc_feature 1..2356: note = Synthetic polynucleotide
  source 1..2356: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaccgccaccatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaatgaatgctttatttgtgaaatttgtgatgctattgctttatttgtaaccattataagctgcaataaacaagttaacaacaacaattgcattcattttatgtttcaggttcagggggaggtgtgggaggttttttaaagcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 57 - DNA, 4452 bp, synthetic construct
  misc_feature 1..4452: note = Synthetic polynucleotide
  source 1..4452: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcttcaggagcagttgtgcgaatagctggagaacaccaggctggatttaaacccagatcgctcttacatttgctctttacctgctgtgctcagcgttcacgtgccctctagctgtagttttctgaagtcagcgcacagcaaggcagtgtgcttagaggttaacagaagggaaaacaacaacaacaaaaatctaaatgagaatcctgactgtttcagctgggggtaaggggggcggattattcatataattgttataccagacggtcgcaggcttagtccaattgcagagaactcgcttcccaggcttctgagagtcccggaagtgcctaaacctgtctaatcgacggggcttgggtggcccgtcgctccctggcttcttccctttacccagggcgggcagcgaagtggtgcctcctgcgtcccccacaccctccctcagcccctcccctccggcccgtcctgggcaggtgacctggagcatccggcaggctgccctggcctcctgcgtcaggacaacgcccacgaggggcgttactgtgcggagatgcaccacgcaagagacaccctttgtaactctcttctcctccctagtgcgaggttaaaaccttcagccccacgtgctgtttgcaaacctgcctgtacctgaggccctaaaaagccagagacctcactcccggggagccagcatgtccactgcggtcctggaaaacccaggcttgggcaggaaactctctgactttggacaggtgagccacggcagcctgagctgctcagttaggggaatttgggcctccagagaaagagatctgaagactgctggtgcttcctggtttcataagctcagtaagaagtctgaattcgttggaagctgatgagaatatccaggaagtcaacagacaaatgtcctcaacaattgtttctaagtaggagaacatctgtcctcggtggctttcacaggaaccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaccgccaccatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagtgtgcgttacgacccttatacccagaggatcgaggtgctggataatacacagcagctgaagatcctggctgactcaatcaatagcgaaatcggaatcctgtgctccgccctgcagaaaatcaaatgagaattcaaggcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagctttacgtacgatcgtcgactgggatgggatgtggaatccttctagatttcttttgtaatatttataaagtgctctcagcaaggtatcaaaatggcaaaattgtgagtaactatcctcctttcattttgggaagaagatgaggcatgaagagaattcagacagaaacttactcagaccaggggaggcagaaactaagcagagaggaaaatgaccaagagttagccctgggcatggaatgtgaaagaaccctaaacgtgacttggaaataatgcccaaggtatattccattctccgggatttgttggcattttcttgaggtgaagaattgcagaatacattctttaatgtgacctacatatttacccatgggaggaagtctgctcctggactcttgagattcagtcataaagcccaggccagggaaataatgtaagtctgcaggcccctgtcatcagtaggattagggagaagagttctcagtagaaaacagggaggctggagagaaaagaatggttaatgttaacgttaatataactagaaagactgcagaacttaggactgatttttatttgaatccttaaaaaaaaaatttcttatgaaaatagtacatggctcttaggagacagaacttattgtacagaggaacagcgtgagagtcagagtgatcccagaacaggtcctggctccatcctgcacatagttttggtgctgctggcaatacggtccccacaactgtgggaaggggttaggggcagggatctcatcaggaaagcataggggtttaaagttctttatagagcacttagaagattgagaatccacaaattatattaataacaaacaaagtagtgtcgtgttatatagtaaatgtgaatttgcagacacatttagggaaaagttataattaaaaaaataggctgtatatatacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 58 - DNA, 2521 bp, synthetic construct
  misc_feature 1..2521: note = Synthetic polynucleotide
  source 1..2521: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcaactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaccatgcccccacccaggaccggaagaggcctgctgtggctgggcctggtgctctcttccgtgtgcgtggccctgggaagcgaaacccaggccaacagcacaaccgacgccctgaatgtgctgctgatcattgtggacgatctgagaccctccctgggctgttacggcgacaaactggtgcggtccccaaacatcgaccagctggcctcccactccctgctgttccagaacgccttcgcccagcaggccgtgtgtgcccccagcagggtgagcttcctgaccggcagaagacctgacaccaccaggctgtacgactttaacagctactggcgggtgcacgccggcaatttcagcaccattcctcagtacttcaaggagaatggctacgtgacaatgtccgtgggcaaggtgtttcatcccggcattagctccaaccacaccgacgatagcccatactcctggtccttccccccctaccatccctccagcgagaagtacgagaacaccaaaacctgcagaggccctgacggagagctgcacgccaacctgctgtgccctgtggatgtcctggatgtgcccgaaggtaagggtttaagggatggttggttggtggggtattaatgtttaattacctggagcacctgcctgaaatcactttttttcaggcaccctgccagacaagcagtccacagagcaggccatccagctgctggagaagatgaagacaagcgccagccccttctttctggccgtgggataccacaagcctcacattccattccggtacccaaaagagttccagaagctgtaccctctggaaaacatcaccctggcccctgaccccgaggtgccagacgggctgcctcctgtggcctacaatccctggatggacatcagacagcgggaggatgtgcaggccctgaatatttccgtgccctatgggcccatccctgtggactttcagcggaaaatcagacagtcttactttgccagcgtgtcctacctggacacccaggtgggccgcctgctctcagccctggacgacctgcagctggccaattccaccatcatcgccttcaccagcgatcacggctgggccctgggcgagcacggggagtgggccaaatacagcaacttcgatgtggccacccacgtgcctctgattttttatgtgcccggccggacagccagcctgcccgaggccggggagaagctctttccttacctggaccctttcgactctgccagccagctgatggaacctggcagacagagcatggacctggtggagctggtgagcctcttccccactctggccggcctggctggcctgcaggtgccaccaagatgcccagtgccttctttccacgtggagctgtgtagagagggaaagaacctgctgaagcacttcagatttagagatctggaggaggatccctacctgccaggcaacccaagggagctgatcgcctacagccagtatcccagaccctctgatatcccccagtggaacagcgataagccctccctgaaagacatcaagattatgggctactccatcaggaccattgactaccggtacacagtgtgggtgggcttcaaccccgatgagtttctggccaacttcagcgacatccacgccggcgagctgtattttgtggactccgaccccctgcaggaccacaacatgtacaacgactcccagggcggcgacctgttccagctgctgatgccctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaacctgcaggtctagatacgtagataagtagcatggcgggttaatcattaactacaaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 59 - DNA, 4483 bp, synthetic construct
  misc_feature 1..4483: note = Synthetic polynucleotide
  source 1..4483: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcacacgtgacgcgtccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaggaacgttttattttcccagagacaggctcagggacttctgggtgtagtggctgtgcagggcttcgtggagcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtgtggccagatcctggcaggatctcgcccatccattccaggccttggccaggggcctgccgcacccactggatccagtagttgctgaagatgtggccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggccgcggcacgtgcttaaggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatctccggagggggaggctgctggtgaatattaaccaaggtcaccccagttatcggaggagcaaacaggggctaagtccacctcgagccatggcgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttgtttaaacgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa